(19) United States
(12) Patent Application Publication
(10) Pub. No.: US 2025/0109413 A1
(43) Pub. Date: Apr. 3, 2025

(54) Method for Producing Genetically Modified Cells

(71) Applicants: Rutgers, The State University of New Jersey, New Brunswick, NJ (US); Horizon Discovery Limited, Cambridge (GB)

(72) Inventors: Shengkan Jin, Belle Mead, NJ (US); Juan-Carlos Collantes, Princeton, NJ (US); John Lambourne, Cambridge (GB); Immacolata Porreca, Cambridge (GB); Tommaso Selmi, Cambridge (GB)

(73) Assignees: Rutgers, The State University of New Jersey, New Brunswick, NJ (US); Horizon Discovery Limited, Cambridge (GB)

(21) Appl. No.: 18/681,775

(22) PCT Filed: Aug. 5, 2022
(86) PCT No.: PCT/US22/74625

§ 371 Date: Feb. 6, 2024

Related U.S. Application Data
(60) Provisional application No. 63/203,996, filed on Aug. 6, 2021.

(51) Int. Cl. C12N 15/90; C12N 9/22; C12N 9/78; C12N 15/11

(52) U.S. Cl. CPC C12N 15/90; C12N 9/22; C12N 9/78; C12N 15/11; C12N 2310/20

(57) ABSTRACT

The present disclosure relates to a new modular approach for the generation of genetically modified cells, particularly immune cells and iPSCs, enabling the simultaneous precise editing of defined nucleic acid targets (knock-out) and the introduction of an exogenous sequence of choice at a desired locus (knock-in) using a common Cas9 element.

### CROSS-REFERENCE TO RELATED APPLICATIONS

This application claims priority under 35 U.S.C. § 119(e) to U.S. Provisional Patent Application Ser. No. 63/203,996, filed on Aug. 6, 2021, which is hereby incorporated by reference in its entirety.

### FIELD OF THE INVENTION

The present disclosure relates to new methods, cells, systems, kits, and other aspects of producing genetically engineered cells using the Clustered Interspaced Regularly Short Palindromic Repeat (CRISPR) based gene editing systems for introducing multiple genetic modifications into cells.

### BACKGROUND

The precise, genetic modulation of primary human cells has multiple applications for the treatment of human diseases, including in the fields of immunotherapy, autoimmunity, and enzymopathy. For example, genetic modulation of patient immune cells is an attractive route for therapy owing to the permanency of the changes made to the immune cells and the low risk of rejection of such cells by the patient. One approach for gene editing of immune cells is to use Clustered Regularly Interspaced Short Palindromic Repeat (CRISPR) systems to induce a double stranded break (DSB) within a gene of interest, which is then repaired by the efficient but error-prone non-homologous end joining (NHEJ) pathway or by the less efficient, but high-fidelity homology directed repair (HDR) pathway. The NHEJ repair pathway is the most active repair mechanism and frequently results in small nucleotide insertions or deletions (indels) at the DSB site, causing amino acid deletions, insertions or frameshift mutations leading to premature stop codons or nonsense mutations within the open reading frame (ORF) of the targeted gene. Furthermore, inducing multiple DSBs during multiplexed gene editing procedures can cause undesirable genotoxicity and the formation of potentially oncogenic gross chromosomal translocations. More precise gene editing can be achieved through the use of modified nucleases (e.g., Cas9 nickase) which retain only one active nuclease domain and generate a DNA nick rather than a blunt-ended DSB. The variant Cas9 D10A, a mutant of SpCas9, retains only the HNH nuclease activity and, in the presence of two guide RNAs (gRNAs) targeting opposite DNA strands, creates a staggered DSB, thus increasing target specificity.

Chimeric antigen receptor-T (CAR-T) cell immunotherapy is a novel method that involves the genetic modification of a patient's own T cells to express a CAR specific for a tumor antigen. The method is an individualized treatment involving expansion of the genetically modified cells ex vivo followed by re-infusion back to the patient. This therapy has shown impressive results in hematological cancers, anti-CD19 CAR-T therapies have been approved for the treatment of CD19 positive leukemia or lymphoma (Yescarta™, Kymriah™, Tecartus™, and Breyanzi™) and anti-BCMA CAR-T therapies have also been approved for multiple myeloma (Abecma™). Despite encouraging results in some patients, the application of CAR-T causes a number of acute side effects, such as cytokine release syndrome and neurological toxicities, leading to the death of the patient in some cases.

Long term safety outcomes, such as immunogenicity and adverse effects on genetically modified T-cell growth and development remain a concern with this therapy. Accordingly, there is a need to develop improved CAR-T cell therapies with reduced side effects and health risks for the patient. Additionally, given the level of complexity associated with the personalized approach, which is currently a necessary requirement, there is a need to develop CAR-T cell therapies which are “off the shelf” or allogeneic and address the problems associated with time to treatment, manufacture, quality, and cost.

CARs are typically transduced into the T cells of a patient using randomly integrating vectors, which may result in oncogenic transformation, variegated transgene expression, and transcriptional silencing. Recently, advances in genome editing enable efficient, targeted gene delivery. Directing a CD19-specific CAR to the T-cell receptor a constant (TRAC) locus allows the expression of CAR under control of the endogenous TRAC regulatory elements, which enhances T-cell potency and delays exhaustion.

### SUMMARY

In a first aspect, the disclosure provides methods for making multiple genetic modifications to a cell, the methods comprising introducing into the cell and/or expressing in the cell:
a) a CRISPR system for integrating an exogenous sequence at a first target nucleic acid sequence, the CRISPR system comprising:
   i) a first gRNA and a second gRNA that are complementary to opposite strands of the first target nucleic acid sequence; and
   ii) a donor nucleic acid sequence comprising the exogenous sequence;
b) a base editing system for introducing a genetic modification at a second target nucleic acid sequence, the base editing system comprising:
   i) an RNA scaffold comprising a guide RNA sequence that is complementary to the second target nucleic acid sequence and, a recruiting RNA motif; and
   ii) an effector fusion protein comprising an RNA binding domain capable of binding to the recruiting RNA motif and an effector domain comprising a base modifying enzyme; and
c) an RNA guided nickase capable of interacting with the first and second gRNAs of the CRISPR system and the RNA scaffold of the base editing system; and
   culturing the cell to produce a cell comprising multiple genetic modifications.

In any embodiment, because the RNA guided nickase is capable of interacting with both the CRISPR system and the RNA scaffold of the base editing system, the method may be performed using only one RNA guided nickase (also referred to herein as a single RNA guided nickase or a common RNA guide nickase). This may be advantageous as it reduces the number of components that need to be provided and delivered to the cell.

In some embodiments, the base modifying enzyme has cytosine deamination activity, adenosine deamination activity, DNA methyl transferase activity, or demethylase activity.

In some embodiments, the RNA guided nickase may be a CRISPR Type II or Type V enzyme. In some embodiments, where the RNA guided nickase is a CRISPR Type II enzyme, the enzyme is a Cas9 nickase. In an embodiment, the RNA guided nickase is nCas9 with one or two Uracil Glycosylase Inhibitors (UGIs).

In some embodiments, the first and second gRNAs may be provided as sgRNAs.

In some embodiments, the RNA scaffold used in the methods, cells, systems and kits herein may comprise a tracrRNA. In CRISPR Type II systems, maturation of a precursor crRNA (pre-crRNA) requires involvement of a trans-acting CRISPR (tracr) RNA. However, in CRISPR Type V systems, no tracrRNA has been identified, and pre-crRNA processing is mediated by the Type V effector proteins themselves.

In some embodiments, the RNA scaffold used in the methods, cells, systems and kits herein may be introduced into the cell as chemically synthesized RNA and may comprise one or more chemical modifications.

In some embodiments, the methods, cells, systems, and kits provided herein may utilize one or more recruiting RNA motifs, in some embodiments, located at the 3′ end of the RNA scaffold. The recruiting RNA motif may be an MS2 aptamer, in some embodiments, an MS2 aptamer that has an extended stem, for example, an extended stem comprising 2-24 nucleotides.

In some embodiments, the methods, cells, systems, and kits provided herein may use an effector domain having cytosine deamination activity or cytidine deamination activity (the terms are used interchangeably), for example, a wild type or genetically engineered version of AID, CDA, APOBEC1, APOBEC3A, APOBEC3B, APOBEC3C, APOBEC3D, APOBEC3F, or other APOBEC family enzymes.

In some embodiments, the methods, cells, systems, and kits provided herein may use an effector domain having adenine deamination activity or adenosine deamination activity (the terms are used interchangeably), for example, a wild type or genetically engineered version of ADA, ADAR family enzymes, or tRNA adenosine deaminases.

In some embodiments, the methods, cells, systems, and kits provided herein may use an effector domain having a DNA methyl transferase activity, for example, a wild type or genetically engineered version of Dnmt1, Dnmt3a, or Dnmt3b.

In some embodiments, the methods, cells, systems, and kits provided herein may use an effector domain having a demethylase activity, for example, a wild type or genetically engineered version of Tet1, Tet2, or TDG.

In some embodiments, the methods, cells, systems, and kits provided herein may use a first gRNA and a second gRNA, that are complementary to opposite strands of a TRAC or B2M locus.

In some embodiments, the methods, cells, systems, and kits may use a modular system comprising multiple base editing systems capable of binding to different target nucleic acid sequences to genetically modify multiple different genetic loci.

In some embodiments, the CRISPR system used in the methods herein may introduce a donor nucleic acid sequence comprising a CAR or TCR encoding sequence flanked by homology arms specific to the first target nucleic acid sequence. In some embodiments, the CAR or TCR encoding sequence is integrated at the TRAC or B2M locus. Expression of the CAR or TCR encoding sequence may be driven by the endogenous TRAC or B2M promoter.

In some embodiments, the nucleic acids encoding each of the CRISPR system, the base editing system, and the RNA guided nickase may be introduced into the cell in a single transfection step. In some embodiments, the donor nucleic acid sequence may be introduced into the cell using a viral vector, for example, AAV. Alternatively, the donor nucleic acid sequence may be introduced into the cell in a single transfection step.

In some embodiments, the methods, cells, systems, and kits provided herein involve the base editing system introducing one or more genetic modifications that correct a genetic mutation, inactivate the expression of a gene, change the expression levels of a gene, or change intron-exon splicing. In other embodiments, the genetic modification introduced by the base editing system may be a point mutation, optionally wherein the point mutation introduces a premature stop codon, disrupts a start codon, disrupts a splice site or corrects a genetic mutation. In some embodiments, the guide RNA sequence used in the methods provided herein may include a splice acceptor-splice donor site (SA-SD) sequence.

In some embodiments, the methods, cells, systems, and kits provided herein may target different genes in the cells. For example, the base editing system may introduce genetic modifications that result in reduced expression of any one or more of TRAC, TRBC1, TRBC2, PDCD1, CD52, and B2M.

In some embodiments, the methods, cells, systems, and kits provided herein may be used to provide multiple genetic modifications that occur simultaneously.

In some embodiments, the methods provided herein may be used to modify any cell, in particular immune cells or human pluripotent stem cells (hPSC). Immune cells may include T cells, Natural Killer (NK) cells, B cells, myeloblasts, lymphoblasts, and CD34+ hematopoietic stem and progenitor cells (HSPCs).

In a particular embodiment, the immune cell is a primary T cell.

In a particular embodiment, the cell is an induced pluripotent stem cell (iPSC).

In a second aspect, the present disclosure provides genetically modified cells obtained by the methods described herein. In some embodiments, the genetically modified cells comprise an exogenous CAR or TCR encoding sequence in the endogenous TRAC or B2M locus and at least one point mutation in 3 or more genes. In other embodiments, the genetically modified cells comprise an exogenous CAR or TCR encoding sequence in the endogenous TRAC or B2M locus and at least one point mutation in 3 or more genes selected from the group consisting of TRAC, TRBC1, TRBC2, PDCD1, CD52, and B2M, resulting in the functional knock-out of said genes.

In a third aspect, the disclosure provides allogeneic T-cells obtained by the methods described herein.

In a fourth aspect, the disclosure provides systems for genetically modifying a cell comprising i) the CRISPR system, ii) the base editing system, and iii) the RNA guided nickase, or one or more nucleic acids encoding i), ii), and iii), as described herein, or one or more expression vectors encoding i), ii), and iii), as described herein.

In a fifth aspect, the disclosure provides kits for genetically modifying a cell comprising i) the CRISPR system, ii) the base editing system, and iii) the RNA guided nickase, or one or more nucleic acids encoding i), ii), and iii), as described herein, or one or more expression vectors encoding i), ii), and iii), as described herein. The kits may further comprise one or more components for introducing a nucleic acid or a polypeptide into a host cell. In some embodiments, the one or more components are selected from the group consisting of a viral vector, a non-integrating viral particle, an extracellular vesicle, a nanoparticle, a cell penetrating peptide, and a donor nucleic acid sequence.

### BRIEF DESCRIPTION OF THE DRAWINGS

The disclosed methods, systems, kits, and other aspects will be described with reference to the following figures, wherein:

FIGS. 1A and 1B show an example of a schematic diagram describing the strategy of simultaneous knock-in of an exogenous gene in a desired locus (such as TRAC locus), while knocking-out that desired locus and knocking-out one or more genes by the base editing technology. FIG. 1A shows a schematic diagram describing the strategy of knock-out of a desired locus (such as TRAC) by double nicks introduced by nCas9-UGI-UGI with knock-in of an exogenous gene (such as CAR gene) in that locus. The enzyme in the CRISPR system (nCAS9-UGI-UGI) is directed to the knock-in locus by a first and second gRNA. The donor template DNA for the integration of the exogenous gene is delivered by a viral vector, such as an adeno-associated virus (e.g., AAV6) or delivered by other methods. FIG. 1B shows a schematic diagram describing the strategy for base editing knockout of one or more genes (such as B2M or CD52). The enzyme in the CRISPR system (common RNA guided nickase), which in this example is nCAS9-UGI-UGI is also directed to the specific gene or genes by the RNA scaffold (sgRNA-aptamer) comprising an RNA aptamer linked to the gRNA. The enzyme complexed with sgRNA-aptamers recruits the deaminase component (MCP-Deaminase) of the base editing system to the site where the base conversion is required.

FIG. 2 shows an example of a linear schematic of the CAR construct used in certain embodiments. In this example, the CAR construct comprises an anti-CD19 scFv derived from the FMC63 mouse hybridoma (FMC63 scFV), a portion of the human CD28 molecule (a hinge extracellular part, a transmembrane domain and the entire intracellular domain) (black box in the figure) and the entire domain of CD3-zeta chain.

FIG. 3 shows a schematic diagram of an example of a suitable CD19 CAR delivery strategy. The enzyme in the CRISPR system of the present disclosure induced integration of CD19 CAR into the TRAC locus. The donor construct (AAV6) contained the CAR gene flanked by homology sequences (LHA and RHA). The CD19-CAR gene was integrated into the TRAC exon 1 locus. Once integrated, CAR expression was driven by the endogenous TCRα promoter while the TRAC locus was disrupted. P2A: the self-cleaving Porcine teschovirus 2A sequence. pA: bovine growth hormone PolyA sequence

FIGS. 4A, 4B, 4C, and 4D show the analysis of targeted integration of a GFP coding sequence into the TRAC locus. A pair of synthetic sgRNAs targeting exon 1 of the TRAC locus and a Cas9-UGI-UGI mRNA were co-delivered, via electroporation, into CD3 positive T-cells. This was followed by transduction with the viral vector AAV6-TRAC-GFP where the GFP coding sequence is flanked by HAs to the TRAC locus. Levels of GFP integration and TCRα/β functional knock-out were determined 4-7 days post-delivery by flow cytometry and compared to the cells where no virus was transduced. Control cells (i.e., cells were no Cas9 and sgRNAs were electroporated) were also analyzed. FIG. 4A shows the level of GFP positive cells on the live population. FIG. 4B shows the level of TCRα/β positive cells on the live population. FIG. 4C shows the distribution of TCR−/GFP+, TCR+/GFP−, TCR+/GFP+ and TCR−/GFP+ cell populations on the live population. FIG. 4D shows viability of the cells in the above conditions.

FIGS. 5A, 5B, 5C, and 5D show the analysis of targeted integration of a CD19-CAR coding sequence into the TRAC locus. A pair of synthetic sgRNAs targeting exon 1 of the TRAC locus and a Cas9-UGI-UGI mRNA were co-delivered, via electroporation, into CD3 positive T-cells. This was followed by transduction with the viral vector AAV6-TRAC-CAR where the CD19-CAR coding sequence is flanked by HAs to the TRAC locus. Levels of CAR integration and TCRα/β functional knock-out were determined 4-7 days post-delivery by flow cytometry and compared to the cells where no virus was transduced. Control cells (i.e., cells were no Cas9 and sgRNAs were electroporated) were also analyzed. FIG. 5A shows the level of CAR positive cells on live cells. FIG. 5B shows the level of TCRα/(3 positive cells on live cells. FIG. 5C shows the distribution of TCR−/CAR+, TCR+/CAR−, TCR+/CAR+ and TCR−/CAR+ cell populations on live cells. FIG. 5D shows viability of the cells in the above conditions.

FIGS. 6A, 6B, 6C, and 6D compare the base editing efficiency and functional KO generation of B2M and CD52 genes in untransduced cells and AAV6 transduced cells. A pair of synthetic sgRNAs targeting exon 1 of the TRAC locus, sgRNA-aptamers for base editing targeting of B2M and CD52 and nCas9-UGI-UGI and Apobec1-MCP mRNAs were co-delivered, via electroporation, into CD3 positive T-cells. This was followed by transduction with the viral vector AAV6-TRAC-CAR. At day 4 post-delivery, base editing efficiency and functional knock-out generation for B2M and CD52 were evaluated by Sanger sequencing and flow cytometry, respectively. Control cells (i.e., cells were no Cas9 and sgRNAs were electroporated) were also analysed. FIGS. 6A and 6B show editing efficiency for B2M and CD52, respectively, determined by Sanger sequencing at Day 4 post-delivery. FIGS. 6C and 6D show the percentage of B2M and CD52 positive cells, respectively, on live cells measured by flow cytometry at Day 4 post-delivery.

FIGS. 7A, 7B, and 7C show the simultaneous knock-in of the CAR gene in the TRAC locus and knock-out of TRAC, B2M, and CD52 achieved with the base editing technology of the present disclosure. A pair of synthetic sgRNAs targeting exon 1 of the TRAC locus, sgRNA-aptamers for base editing targeting of B2M and CD52 and nCas9-UGI-UGI and Apobec1-MCP mRNAs were co-delivered, via electroporation, into CD3 positive T-cells. This was followed by transduction with the viral vector AAV6-TRAC-CAR. At day 4 post-delivery functional knock-out generation for B2M, CD52 and TCRα/b and CAR integration were evaluated by flow cytometry. FIG. 7A shows the level of TCRα/b positive cells on live cells measured by flow cytometry. FIG. 7B shows the level of CAR positive cells on live cells measured by flow cytometry. FIG. 7C shows flow cytometry data displaying the fraction of cells KO in one, two, three genes or unedited within the CAR positive population (Single KO (TRAC KO+B2M KO+CD52 KO), double KO (TRAC-B2M KO+TRAC-CD52 KO+B2M-CD52 KO), and triple KO (TRAC-B2M-CD52 KO)).

FIGS. 8A, 8B, 8C, 8D, 8E, and 8F show base editing efficiency by the cytidine base editing technology (FIGS. 8A, 8B, 8C) and efficiency of indels formation by wt Cas9 (FIGS. 8D, 8E, 8F) at the B2M, CD52 and PDCD1 loci in untransduced and AAV6 transduced samples. A pair of synthetic sgRNAs targeting exon 1 of the TRAC locus, sgRNA-aptamers for base editing targeting of B2M, CD52 and PDCD1 and nCas9-UGI-UGI and Apobec1-MCP mRNAs were co-delivered, via electroporation, into CD3 positive T-cells. Cas9 samples have been electroporated with wild type Cas9 mRNA and regular sgRNAs. This was followed by transduction with the viral vector AAV6-TRAC-CAR. At day 4 post-delivery, base editing efficiency and efficiency of indel formation were evaluated by Sanger in untransduced and transduced samples.

FIGS. 9A, 9B, and 9C show functional KO generation of B2M, CD52 and PDCD1 genes by cytidine base editing (nCas9-UGI-UGI/Apobec) and wt Cas9 in untransduced and AAV6 transduced samples. A pair of synthetic sgRNAs targeting exon 1 of the TRAC locus, sgRNA-aptamers for base editing targeting of B2M, CD52, and PDCD1 and nCas9-UGI-UGI and Apobec1-MCP mRNAs were co-delivered, via electroporation, into CD3 positive T-cells. Cas9 samples have been electroporated with wild type Cas9 mRNA and regular sgRNAs. This was followed by transduction with the viral vector AAV6-TRAC-CAR. At day 4 post-delivery, functional knock-out generation for B2M, CD52 and PDCD1 genes were evaluated by flow cytometry (FIGS. 9A, 9B, and 9C respectively). Control samples represent samples that were mock electroporated and left untransduced or transduced with the AAV6-TRAC-CAR.

FIGS. 10A and 10B show the knock-in of the CAR in the TRAC locus and knock-out of TRAC when simultaneously knocking out three more genes. A pair of synthetic sgRNAs targeting exon 1 of the TRAC locus, sgRNA-aptamers for base editing targeting of B2M, CD52 and PDCD1 and nCas9-UGI-UGI and Apobec1-MCP mRNAs were co-delivered, via electroporation, into CD3 positive T-cells. Cas9 samples have been electroporated with wildtype Cas9 mRNA and regular sgRNAs. This was followed by transduction with the viral vector AAV6-TRAC-CAR. Levels of CAR integration and TCRα/b functional knock-out were determined 4-7 days post-delivery by flow cytometry. FIG. 10A shows the level of CAR positive cells on live cells. FIG. 10B shows the level of TCRα/b positive cells on live cells.

FIG. 11 shows tumor killing potential of CAR-T cells generated with the base editing technology. For the generation of CAR-T cells, a pair of synthetic sgRNAs targeting exon 1 of the TRAC locus, sgRNA-aptamers for base editing of B2M, CD52, and PDCD1 genes and nCas9-UGI-UGI and Apobec1-MCP mRNAs were co-delivered, via electroporation, into CD3 positive T-cells. Cas9 samples have been electroporated with wild type Cas9 mRNA and regular sgRNAs. This was followed by transduction with the viral vector AAV6-TRAC-CAR. Around 7 days post electroporation CD3+ cells were depleted from the culture and the resulting allogeneic CAR-T cells were incubated with CD19 positive Raji cells, previously loaded with Calcein AM, for 4 hours at 1:1 and 5:1 CAR-T:Raji cells ratio. After the incubation, culture medium was collected and analyzed for fluorescence emission as measure of Raji cell lysis. The percentage of target cell killing is calculated as [(average of test condition−average of negative control condition)/(average of positive control condition−average of negative control condition)]*100 where negative control condition is Raji cells without CAR-T cells and positive control condition is Raji cells exposed to 2% triton to achieve complete lysis.

FIG. 12 shows an example of a linear schematic of the scHLA-E trimer used in certain embodiments. In this example, the scHLA-E trimer construct comprises the leader peptide of human B2M (hB2M I.p.), a HLA-E-binding peptide antigen, a 15 amino acid linker ((G4S)3), the mature human B2M (hB2M), a 20 amino acid linker ((G4S)4) and the mature HLA-E heavy chain

FIG. 13 shows an example of the schematic for the circular double-stranded DNA used in certain embodiments. The exogenous DNA template was flanked by homology arms from the B2M locus (right homology arm, RHA and left homology arm, LHA). In the circular form, the exogenous DNA template with homology arms was flanked (A) or not (B) on both sides by the sequence of the gRNA pair that target the B2M genomic locus (CTS or CRISPR/Cas9 target sequences or sgRNAs B2M targeting sequences) so that once the circular double-stranded DNA is co-delivered in the cells together to the CRISPR components, the donor nucleic acid sequence was released as linear DNA from the circular dsDNA following the cut by CRISPR/Cas. pA: bovine growth hormone PolyA sequence

FIG. 14 shows a schematic diagram of an example of a suitable scHLA-E trimer delivery strategy. The enzyme in the CRISPR system of the present disclosure induced integration of scHLA-E trimer into the B2M locus. The exogenous DNA template contained the scHLA-E trimer coding sequence flanked by homology sequences (LHA and RHA). The scHLA-E trimer transgene was integrated into the B2M exon 1 locus. Once integrated, scHLA-E trimer expression was driven by the endogenous B2M promoter while the B2M locus was disrupted. pA: bovine growth hormone PolyA sequence

FIGS. 15A, 15B, 15C, and 15D shows the analysis of targeted integration of a tGFP coding sequence into the B2M locus and base editing at the CIITA locus when delivering the exogenous DNA template as circular double-stranded DNA. A pair of synthetic sgRNAs targeting exon 1 of the B2M locus, sgRNA-aptamers for base editing targeting of CIITA, nCas9-UGI-UGI, and Apobec1-MCP mRNAs and circular double-stranded DNA containing the GFP coding sequence with homology arms to the B2M gene were co-delivered, via electroporation, into iPSCs. In the circular form, the exogenous DNA template with homology arms was flanked or not on both sides by sgRNAs B2M targeting sequences (CTS_B2M_tGFP and B2M_tGFP respectively in the graphs) (CTS stands for CRISPR/Cas9 target sequences). Levels of GFP integration were determined after 48 hours of treatment with interferon-y 5-7 days post-delivery by flow cytometry and compared to the cells that did not receive the exogenous DNA template. Base editing efficiency at the CIITA locus was evaluated by Sanger sequencing at 5-7 days post-delivery. Control cells (i.e., cells where no Cas9 and sgRNAs were electroporated) were also analyzed. FIG. 15A shows base editing efficiency for CIITA gene determined by Sanger sequencing. FIG. 15B shows the level of B2M positive cells on live cells. FIG. 15C shows the level of GFP positive cells on live cells. FIG. 15D shows the distribution of GFP−/B2M+, GFP−/B2M+, GFP+/B2M+, and GFP+/B2M− cell populations on live cells.

FIGS. 16A, 16B, 16C, and 16D show the analysis of targeted integration of a tGFP coding sequence into the B2M locus and base editing at the CIITA gene when delivering the exogenous DNA template as linear double-stranded DNA. A pair of synthetic sgRNAs targeting exon 1 of the B2M locus, sgRNA-aptamers for base editing targeting of CIITA gene, nCas9-UGI-UGI and Apobec1-MCP mRNAs and linear double-stranded DNA containing the tGFP coding sequence with homology arms to the B2M gene were co-delivered, via electroporation, into iPSCs. In the linear form, the exogenous DNA template with homology arms was flanked or not on both sides by sgRNAs B2M targeting sequences (linear CTS_B2M_tGFP and linear B2M_tGFP respectively in the graphs) (CTS stands for CRISPR/Cas9 target sequences). Level of GFP integration was determined after 48 hours of treatment with interferon-y 5-7 days post-delivery by flow cytometry and compared to the cells that did not receive the exogenous DNA template. Base editing efficiency at the CIITA gene was evaluated by Sanger sequencing at 5-7 days post-delivery. Control cells (i.e., cells where no Cas9 and sgRNAs were electroporated) were also analyzed. FIG. 16A shows base editing efficiency for CIITA determined by Sanger sequencing. FIG. 16B shows the level of B2M positive cells on live cells. FIG. 16C shows the level of GFP positive cells on live cells. FIG. 16D shows the distribution of GFP−/B2M+, GFP−/B2M+, GFP+/B2M+, and GFP+/B2M− cell populations on live cells.

FIGS. 17A, 17B, and 17C show the analysis of targeted integration of a scHLA-E trimer coding sequence into the B2M locus and base editing at the CIITA gene when delivering the exogenous DNA template as circular double-stranded DNA. A pair of synthetic sgRNAs targeting exon 1 of the B2M locus, sgRNA-aptamers for base editing targeting of CIITA, nCas9-UGI-UGI and Apobec1-MCP mRNAs and circular double-stranded DNA containing the scHLA-E trimer coding sequence with homology arms to the B2M gene were co-delivered, via electroporation, into iPSCs. In the circular form, the exogenous DNA template with homology arms was flanked or not on both sides by sgRNAs B2M targeting sequences (linear CTS_B2M_scHLA-E_trimer and linear B2M_scHLA-E_trimer respectively in the graphs) (CTS stands for CRISPR/Cas9 target sequences). Level of scHLA-E_trimer integration was determined after 48 hours of treatment with interferon-y 5-7 days post-delivery by flow cytometry and compared to the cells that did not receive the exogenous DNA template. Base editing efficiency at the CIITA gene was evaluated by Sanger sequencing at 5-7 days post-delivery. Control cells (i.e., cells where no Cas9 and sgRNAs were electroporated) were also analyzed. FIG. 17A shows base editing efficiency for CIITA gene determined by Sanger sequencing. FIG. 17B shows the level of B2M positive cells on live cells. FIG. 17C shows the level of scHLA-E_trimer positive cells on live cells.

### DETAILED DESCRIPTION

The present disclosure relates to a new modular approach for the generation of genetically modified cells, particularly immune cells and iPSCs, enabling the simultaneous precise editing of defined nucleic acid targets (knock-out) and the introduction of an exogenous sequence of choice at a desired locus (knock-in) using a common Cas9 element.

The present inventors have developed a new modular methodology for the generation of genetically modified cells, particularly immune cells and iPSCs which enables the simultaneous, precise editing of defined nucleic acid targets (knock-out) and the introduction of a chosen exogenous sequence at a desired locus (knock-in) using a common CRISPR/Cas9 targeting element. Advantageously, it has been shown herein that the methods and systems according to the present disclosure can be used to simultaneously knock-in an exogenous gene, such as a CAR or TCR, and base edit multiple genes to produce functional knock-outs.

The methods provided herein may target different genes in cells, in particular immune cells. For example, the base editing components used in the method can be used to introduce genetic modifications that result in a desired base change resulting in the subsequent phenotypic loss of any of the following proteins encoded by the genes TRAC, TRBC1, TRBC2, PDCD1, CD52, CIITA, NKG2A and B2M. The methods may be used to edit one or both alleles of a target gene in a cell for example, an immune or iPS cell. The methods provided herein may be used to edit multiple different genes (multiplex base editing), and successfully edit one or both alleles of the target genes. For example, the method may use multiple RNA scaffolds comprising different guide RNA sequences to genetically modify (base edit) multiple different genetic loci, (e.g., 2 to 10). Advantageously, it has been shown herein that the methods and systems can also be used to simultaneously knock-in an exogenous gene, such as a CAR or TCR encoding sequence, and base edit multiple genes to produce functional knock-outs.

The methods according to the present disclosure can be configured to produce genetically engineered cells, particularly immune cells, and stem cells and progenitor cells that can be differentiated into immune cells. Immune cells include T cells, Natural Killer (NK) cells, B cells, myeloblasts, lymphoid dendritic cells, myeloid dendritic cells, macrophages, eosinophils, neutrophils, basophils and CD34+ hematopoietic stem and progenitor cells (HSPCs). HSPCs can give rise to common myeloid and common lymphoid progenitors which can differentiate into T cells, dendritic cells, Natural Killer (NK) cells, B cells, myeloblasts, and other immune cells, erythroblasts, megakaryoblasts and mast cells. In addition, pluripotent stem cells derived from human sources hPSCs (human pluripotent stem cells) which include hESCs (human embryonic stem cells) and induced pluripotent stem cells (iPSCs), can be used to derive immune cells. hPSCs and for instance, IPSCs can be genetically engineered prior to being differentiated into populations of desired cell types, or the iPSCs can be differentiated into populations of desired cell types and then subsequently genetically engineered.

In some embodiments, immune cells are T cells, such as CAR-T/TCR-T cells. Genetically engineered T cells may be derived from primary T cells or differentiated from stem cells that are suitable as “universally acceptable” cells for therapeutic application. Suitable stem cells include, but are not limited to, mammalian stem cells such as human stem cells, including, but not limited to, hematopoietic stem cells (HSC), embryonic and induced pluripotent stem cells (iPSC), derived from neural, mesenchymal, mesodermal, liver, pancreatic, muscle, and retinal stem cells. Other stem cells include, but are not limited to, mammalian stem cells such as mouse stem cells, e.g., mouse embryonic stem cells.

The CRISPR based platform of the present disclosure can be used to integrate an exogenous DNA sequence into one or more target nucleic acid sequences of a cell, in particular a T cell or a iPSC. The exogenous DNA can comprise a CAR or TCR sequence, or may code for a therapeutic protein or correct a point mutation/indel in the genome.

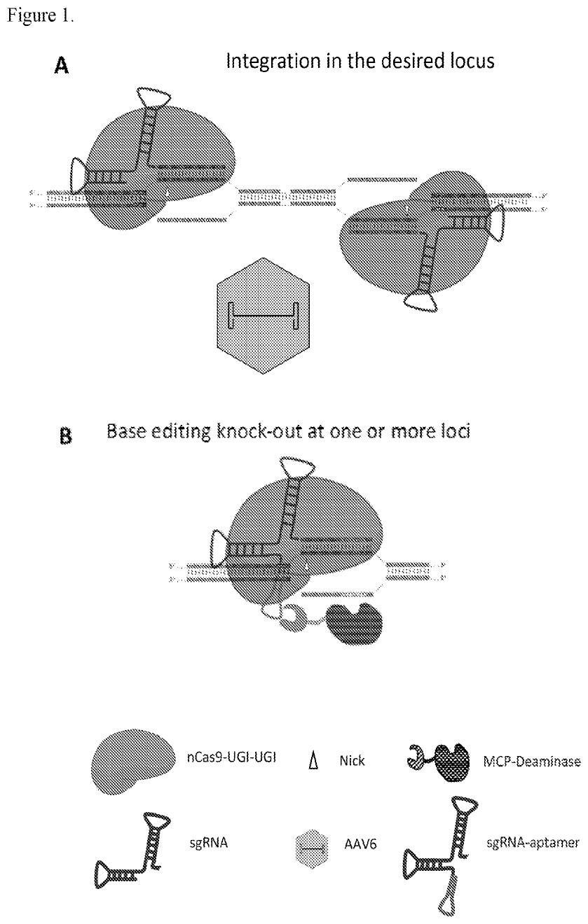

In an embodiment, the present disclosure is based on the application of the CRISPR based platform for the generation of CAR-T cells which have one or more site directed mutations resulting in functional ablation of target genes (FIG. 1). The system may be used in a multiplex manner to generate CAR-T cells with advantageous properties such as prevention of immunosuppressive side effects, graft vs. host and host vs. graft disease. The present disclosure may be particularly relevant for the development of allogeneic, off-the-shelf therapies.

### Definitions

As used herein, the term “about” refers to +/−10%.

The term “antisense,” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation.

“Cell,” as defined herein, comprises any type of cell, prokaryotic or a eukaryotic cell, isolated or not, cultured or not, differentiated or not, and comprising also higher level organizations of cells such as tissues, organs, organisms or parts thereof. Exemplary cells include, but are not limited to vertebrate cells, mammalian cells, human cells, plant cells, animal cells, invertebrate cells, nematodal cells, insect cells, stem cells, and the like.

“Complement” or “complementary” as used herein means Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules. Partial complementary may mean less than 100% complementarity, for example 80% complementarity. “Complementary”, as used herein, means that a first sequence is at least 60%>, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Delivery vector” or“delivery vectors” is directed to any delivery vector which can be used in the present invention to put into cell contact or deliver inside cells or subcellular compartments agents/chemicals and molecules (proteins or nucleic acids) needed in the present invention. It includes, but is not limited to, transducing vectors, liposomal delivery vectors, plasmid delivery vectors, viral delivery vectors, bacterial delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, nucleic acid(s), proteins), or other vectors such as plasmids and T-DNA. These delivery vectors are molecule carriers.

“Donor nucleic acid” is defined here as any nucleic acid supplied to an organism or receptacle to be inserted or recombined wholly or partially into the target sequence either by DNA repair mechanisms, homologous recombination (HR), or by non-homologous end-joining (NHEJ).

“Gene” as used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

“Gene targeting” is used herein as any genetic technique that induces a permanent change to a target nucleic acid sequence including deletion, insertion, mutation, and replacement of nucleotides in a target sequence.

“Target nucleic acid” or “target sequence” as used herein is any desired predetermined nucleic acid sequence to be acted upon, including but not limited to coding or non-coding sequences, genes, exons or introns, regulatory sequences, intergenic sequences, synthetic sequences and intracellular parasite sequences. In some embodiments, the target nucleic acid resides within a target cell, tissue, organ or organism. The target nucleic acid comprises a target site, which includes one or more nucleotides within the target sequence, which are modified to any extent by the methods and compositions disclosed herein. For example, the target site may comprise one nucleotide. For example, the target site may comprise 1-300 nucleotides. For example, the target site may comprise about 1-100 nucleotides. For example, the target site may comprise about 1-50 nucleotides. For example, the target site may comprise about 1-35 nucleotides. In some embodiments, a target nucleic acid may include more than one target site, that may be identical or different,

“Genomic or genetic modification” is used herein as any modification generated in a genome or a chromosome or extra-chromosomal DNA or organellar DNA of an organism as the result of gene targeting or gene-functional modification.

“Mutant” as used herein refers to a sequence in which at least a portion of the functionality of the sequence has been lost, for example, changes to the sequence in a promoter or enhancer region will affect at least partially the expression of a coding sequence in an organism. As used herein, the term “mutation,” refers to any change in a sequence in a nucleic acid sequence that may arise such as from a deletion, addition, substitution, or rearrangement. The mutation may also affect one or more steps that the sequence is involved in. For example, a change in a DNA sequence may lead to the synthesis of an altered mRNA and/or a protein that is active, partially active or inactive.

An “exogenous” sequence, as used herein, refers to a sequence that is not normally present in the genome of a specific cell, but can be introduced into a cell by the method of the disclosure.

The term “% indel,” as used herein, refers to the percentage of insertions or deletions of several nucleotides in the target sequence of the genome.

As used herein, the term “variant” refers to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example, polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 86%, about 87%, about 88% about 89%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example, Western blot. Generally, a variant of a polypeptide, can have at least about 60%, about 65%, about 70%, about 75%, about 80%, about 85%, about 86%, about 87%, about 88% about 89%, about 90%, about 91%, about 92%, about 93%, about 94%, about 95%, about 96%, about 97%, about 98%, about 99% or more sequence identity to the reference polypeptide as determined by sequence alignment programs known by skilled artisans.

Exogenous Sequences to be Integrated (e.g., CAR or scHLA-E)

In some embodiments, the donor nucleic acid sequence comprising the exogenous sequence is a sequence encoding a protein of interest. In some embodiments, the donor nucleic acid sequence is selected from the group consisting of CAR nucleic acid construct, TCR nucleic acid, and scHLA-E.

The “chimeric antigen receptor” (CAR) is sometimes called a “chimeric receptor,” a “T-body,” or a “chimeric immune receptor” (CIR). As used herein, the term “chimeric antigen receptor” (CAR) refers to an artificially constructed hybrid protein or polypeptide comprising extracellular antigen binding domains of an antibody (e.g., single chain variable fragment (scFv)) operably linked to a transmembrane domain and at least one intracellular domain. Generally, the antigen binding domain of a CAR has specificity for a particular antigen expressed on the surface of a target cell of interest. For example, T cells can be engineered to express CAR specific for CD19 on B-cell lymphoma.

First generation CAR constructs comprise a binding domain (a scFv antibody), a hinge region, a transmembrane domain and an intracellular signalling domain (Liu et al., 2019, Frontiers in Immunology, the entire contents of which are incorporated herein by reference).

Yescarta™ (Axicabtagene ciloleucel) was approved for use in 2017 for the treatment of large B-cell lymphoma that has failed conventional treatment and was one of the first therapies of this type. It employs a binding domain that targets CD19, a protein expressed by normal B cells, B cell leukemias, and lymphomas. The second generation CAR (Kochenderfer et al. 2009, J Immunotherapy, the entire contents of which are incorporated herein by reference) used in this therapy consists of an anti-CD19 scFv derived from the FMC63 mouse hybridoma (Nicholson et al 1997, Mol Immunology, the entire contents of which are incorporated herein by reference), a portion of the human CD28 molecule (a hinge extracellular part, a transmembrane domain and the entire intracellular domain) and the entire domain of CD3-zeta chain.

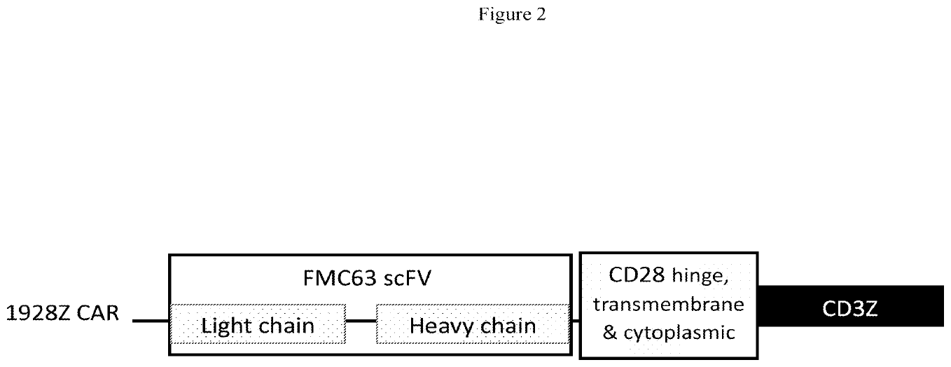

In some embodiments, the exogenous sequence integrated into the target nucleic acid sequence comprises a CAR encoding sequence. In some embodiments, the CAR construct comprises a binding domain, a hinge region, a transmembrane domain and an intracellular signaling domain. The CAR construct used in some of the embodiments of the present disclosure is depicted in FIG. 2.

In some embodiments, the binding domain is a scFv antibody. In some embodiments, the scFv antibody comprises an anti-CD19 scFv derived from the FMC63 mouse hybridoma (FMC63 scFV). In a particular embodiment, the binding domain is an anti-CD19 scFv. In another embodiment, the binding domain is an anti-B-cell maturation antigen (BCMA) scFv.

In some embodiments, the CAR construct comprises a portion of the human CD28 molecule (for example, a hinge extracellular part, a transmembrane domain, and the entire intracellular domain).

In some embodiments, the CAR construct comprises the entire domain of CD3-zeta chain.

In some embodiments, the exogenous sequence integrated into the target nucleic acid sequence comprises a CAR encoding sequence comprising a FMC63 scFV, a CD28 hinge extracellular part, a transmembrane domain and the entire intracellular domain and a CD3Z chain.

The intracellular signaling domain effects signaling inside the cell via phosphorylation of CD3-zeta following antigen binding. CD3-zeta's cytoplasmic domain is routinely used as the main CAR endodomain component. Other co-stimulatory molecules in addition to CD3 signaling are also required for T cell activation and so CAR receptors typically include co-stimulatory molecules including CD28, CD27, CD134 (Ox40) and CD137 (4-1BB).

Examples of first, second, third, and fourth generation CAR are described in Subklewe M et al, Transfusion Medicine and Hemotherapy. 2019 February; 46(1):15-24, the contents of which are incorporated herein by reference in their entireties. In some embodiments, the exogenous sequence is a CAR sequence of first, second, third or fourth generation CAR.

In a particular embodiment, the intracellular signaling domain is the entire intracellular domain of CD3-zeta chain. In one embodiment, the intracellular signaling domain additionally comprises 41BB-CD3-zeta chain or CD28-CD3-zeta chain.

The hinge region is typically a small structural spacer that sits between the binding domain and the cells outer membrane. Ideally, it enhances the flexibility of the scFv to reduce spacial constraints between the CAR and its target antigen. Design of the hinge region has been described in the art and typically is based on sequences which are membrane proximal regions from other immune molecules such as IgG, CD8 and CD28 (Chandran, S S et al. 2019, Immunological Reviews, 290 (1):127-147 and Qin L, et al. 2017, Journal of Hematological Oncology. 10 (1) 68, the contents of which are incorporated herein by reference in their entireties).

The transmembrane domain is a structural component consisting of a hydrophobic alpha helix that spans the cell membrane. It functions by anchoring the CAR to the plasma membrane thereby bridging the hinge region and binding domain with the intracellular signaling domain. The CD28 transmembrane domain is typically used in CARs and is known to result in a stably expressed receptor.

In some embodiments, the CAR nucleic acid construct comprises a binding domain that targets CD19 and an intracellular signaling domain which comprises the entire intracellular domain of CD3-zeta chain and a portion of a CD28 co-stimulatory molecule.

CAR T cell genetic modification may occur via viral-based gene transfer methods or by non-viral methods such as DNA-based transposons, CRISPR/Cas9 technology or direct transfer of in vitro transcribed mRNA by electroporation. Gene transfer technologies either integrate at specific loci of interest or they are randomly, or pseudo-randomly, integrated into the genome. The random or pseudo-random genome integration gene transfer methods include, but are not limited to, methods such as transposons, lentivirus, retrovirus, and adenovirus. Locus specific integration technology offers the advantage of being more predictable, with the possibility to replace regions of the genome and precisely insert exogenous genetic material. In some embodiments, the CAR nucleic acid is integrated into the genome or a chromosome or extra-chromosomal DNA or organellar DNA of an organism as the result of gene targeting.

In other embodiments, the donor nucleic acid sequence is a TCR gene.

In other embodiments, the donor nucleic acid sequence is a scHLA-E trimer. The scHLA-E trimer is a chimeric protein that comprises the following elements: (a) the leader peptide of B2M, (b) VMAPRTLIL (a HLA-E-binding peptide SEQ ID NO: 1), (c) a 15 amino acid linker (G4S)3, (d) a mature human B2M, (e) a 20 amino acid linker (G4S)4 and (f) a mature HLA-E heavy chain.

In some embodiments, scHLA-E trimer nucleic acid sequence comprises the sequence set forth in accession number AY289236.1 (SEQ ID NO: 2):

AAGCTTTGAGCCGAGATGTCTCGCTCCGTGGCCTTAGCTGTGCTCGCGCT
ACTCTCTCTTTCTGGCCTCGAGGCTGTTATGGCTCCGCGGACTTTAATTT
TAGGTGGTGGCGGATCCGGTGGTGGCGGTTCTGGTGGTGGCGGCTCCATC
CAGCGTACGCCAAAGATTCAGGTTTACTCACGTCATCCAGCAGAGAATGG
AAAGTCAAATTTCCTGAATTGCTATGTGTCTGGGTTTCATCCATCCGACA
TTGAAGTTGACTTACTGAAGAATGGAGAGAGAATTGAAAAAGTGGAGCAT
TCAGACTTGTCTTTCAGCAAGGACTGGTCTTTCTATCTCTTGTACTACAC
TGAATTCACCCCCACTGAAAAAGATGAGTATGCCTGCCGTGTGAACCATG
TGACTTTGTCACAGCCCAAGATAGTTAAGTGGGATCGCGACATGGGTGGT
GGCGGTTCTGGTGGTGGCGGTAGTGGCGGCGGAGGAAGCGGTGGTGGCGG
TTCCGGATCTCACTCCTTGAAGTATTTCCACACTTCCGTGTCCCGGCCCG
GCCGCGGGGAGCCCCGCTTCATCTCTGTGGGCTACGTGGACGACACCCAG
TTCGTGCGCTTCGACAACGACGCCGCGAGTCCGAGGATGGTGCCGCGGGC
GCCGTGGATGGAGCAGGAGGGGTCAGAGTATTGGGACCGGGAGACACGGA
GCGCCAGGGACACCGCACAGATTTTCCGAGTGAACCTGCGGACGCTGCGC
GGCTACTACAATCAGAGCGAGGCCGGGTCTCACACCCTGCAGTGGATGCA
TGGCTGCGAGCTGGGGCCCGACAGGCGCTTCCTCCGCGGGTATGAACAGT
TCGCCTACGACGGCAAGGATTATCTCACCCTGAATGAGGACCTGCGCTCC
TGGACCGCGGTGGACACGGCGGCTCAGATCTCCGAGCAAAAGTCAAATGA
TGCCTCTGAGGCGGAGCACCAGAGAGCCTACCTGGAAGACACATGCGTGG
AGTGGCTCCACAAATACCTGGAGAAGGGGAAGGAGACGCTGCTTCACCTG
GAGCCCCCAAAGACACACGTGACTCACCACCCCATCTCTGACCATGAGGC
CACCCTGAGGTGCTGGGCTCTGGGCTTCTACCCTGCGGAGATCACACTGA
CCTGGCAGCAGGATGGGGAGGGCCATACCCAGGACACGGAGCTCGTGGAG
ACCAGGCCTGCAGGGGATGGAACCTTCCAGAAGTGGGCAGCTGTGGTGGT
GCCTTCTGGAGAGGAGCAGAGATACACGTGCCATGTGCAGCATGAGGGGC
TACCCGAGCCCGTCACCCTGAGATGGAAGCCGGCTTCCCAGCCCACCATC
CCCATCGTGGGCATCATTGCTGGCCTGGTTCTCCTTGGATCTGTGGTCTC
TGGAGCTGTGGTTGCTGCTGTGATATGGAGGAAGAAGAGCTCAGGTGGAA
AAGGAGGGAGCTACTATAAGGCTGAGTGGAGCGACAGTGCCCAGGGGTCT
GAGTCTCACAGCTTGTAATCTAGA

In some embodiments, the AY289236.1 sequence is modified in specific nucleotides to avoid recognition by the sgRNA pair used to target the endogenous locus. The transgene is also flanked by homology arms from the locus where it is integrated (e.g., B2M homology arms), surrounding the CRISPR/Cas9 cleavage site. The resulting sequence aimed to integrate in the B2M locus is referred to as scHLA-E_trimer B2M-900HAs (SEQ ID NO: 3):

TAATTCATTCATTCATCCATCCATTCGTTCATTCGGTTTACTGAGTACCT
ACTATGTGCCAGCCCCTGTTCTAGGGTGGAAACTAAGAGAATGATGTACC
TAGAGGGCGCTGGAAGCTCTAAAGCCCTAGCAGTTACTGCTTTTACTATT
AGTGGTCGTTTTTTTCTCCCCCCCGCCCCCCGACAAATCAACAGAACAAA
GAAAATTACCTAAACAGCAAGGACATAGGGAGGAACTTCTTGGCACAGAA
CTTTCCAAACACTTTTTCCTGAAGGGATACAAGAAGCAAGAAAGGTACTC
TTTCACTAGGACCTTCTCTGAGCTGTCCTCAGGATGCTTTTGGGACTATT
TTTCTTACCCAGAGAATGGAGAAACCCTGCAGGGAATTCCCAAGCTGTAG
TTATAAACAGAAGTTCTCCTTCTGCTAGGTAGCATTCAAAGATCTTAATC
TTCTGGGTTTCCGTTTTCTCGAATGAAAAATGCAGGTCCGAGCAGTTAAC
TGGCTGGGGCACCATTAGCAAGTCACTTAGCATCTCTGGGGCCAGTCTGC
AAAGCGAGGGGGCAGCCTTAATGTGCCTCCAGCCTGAAGTCCTAGAATGA
GCGCCCGGTGTCCCAAGCTGGGGCGCGCACCCCAGATCGGAGGGCGCCGA
TGTACAGACAGCAAACTCACCCAGTCTAGTGCATGCCTTCTTAAACATCA
CGAGACTCTAAGAAAAGGAAACTGAAAACGGGAAAGTCCCTCTCTCTAAC
CTGGCACTGCGTCGCTGGCTTGGAGACAGGTGACGGTCCCTGCGGGCCTT
GTCCTGATTGGCTGGGCACGCGTTTAATATAAGTGGAGGCGTCGCGCTGG
CGGGCATTCCTGAAGCTGACAGCATTCGGGACGAGATGTCTCGCTCAGTC
GCCTTAGCTGTGCTCGCGCTACTCTCTCTGTCCGGGCTCGAAGCTGTTAT
GGCTCCGCGGACTTTAATTTTAGGTGGTGGCGGATCCGGTGGTGGCGGTT
CTGGTGGTGGCGGCTCCATCCAGCGTACGCCAAAGATTCAGGTTTACTCA
CGTCATCCAGCAGAGAATGGAAAGTCAAATTTCCTGAATTGCTATGTGTC
TGGGTTTCATCCATCCGACATTGAAGTTGACTTACTGAAGAATGGAGAGA
GAATTGAAAAAGTGGAGCATTCAGACTTGTCTTTCAGCAAGGACTGGTCT
TTCTATCTCTTGTACTACACTGAATTCACCCCCACTGAAAAAGATGAGTA
TGCCTGCCGTGTGAACCATGTGACTTTGTCACAGCCCAAGATAGTTAAGT
GGGATCGCGACATGGGTGGTGGCGGTTCTGGTGGTGGCGGTAGTGGCGGC
GGAGGAAGCGGTGGTGGCGGTTCCGGATCTCACTCCTTGAAGTATTTCCA
CACTTCCGTGTCCCGGCCCGGCCGCGGGGAGCCCCGCTTCATCTCTGTGG
GCTACGTGGACGACACCCAGTTCGTGCGCTTCGACAACGACGCCGCGAGT
CCGAGGATGGTGCCGCGGGCGCCGTGGATGGAGCAGGAGGGGTCAGAGTA
TTGGGACCGGGAGACACGGAGCGCCAGGGACACCGCACAGATTTTCCGAG
TGAACCTGCGGACGCTGCGCGGCTACTACAATCAGAGCGAGGCCGGGTCT
CACACCCTGCAGTGGATGCATGGCTGCGAGCTGGGGCCCGACAGGCGCTT
CCTCCGCGGGTATGAACAGTTCGCCTACGACGGCAAGGATTATCTCACCC
TGAATGAGGACCTGCGCTCCTGGACCGCGGTGGACACGGCGGCTCAGATC
TCCGAGCAAAAGTCAAATGATGCCTCTGAGGCGGAGCACCAGAGAGCCTA
CCTGGAAGACACATGCGTGGAGTGGCTCCACAAATACCTGGAGAAGGGGA
AGGAGACGCTGCTTCACCTGGAGCCCCCAAAGACACACGTGACTCACCAC
CCCATCTCTGACCATGAGGCCACCCTGAGGTGCTGGGCTCTGGGCTTCTA
CCCTGCGGAGATCACACTGACCTGGCAGCAGGATGGGGAGGGCCATACCC
AGGACACGGAGCTCGTGGAGACCAGGCCTGCAGGGGATGGAACCTTCCAG
AAGTGGGCAGCTGTGGTGGTGCCTTCTGGAGAGGAGCAGAGATACACGTG
CCATGTGCAGCATGAGGGGCTACCCGAGCCCGTCACCCTGAGATGGAAGC
CGGCTTCCCAGCCCACCATCCCCATCGTGGGCATCATTGCTGGCCTGGTT
CTCCTTGGATCTGTGGTCTCTGGAGCTGTGGTTGCTGCTGTGATATGGAG
GAAGAAGAGCTCAGGTGGAAAAGGAGGGAGCTACTATAAGGCTGAGTGGA
GCGACAGTGCCCAGGGGTCTGAGTCTCACAGCTTGTAAtaaacccgctga
tcagcctcgactgtgccttctagttgccagccatctgttgtttgcccctc
ccccgtgccttccttgaccctggaaggtgccactcccactgtcctttcct
aataaaatgaggaaattgcatcgcattgtctgagtaggtgtcattctatt
ctggggggtggggtggggcaggacagcaagggggaggattgggaagacaa
tagcaggcatgctggggatgcggtgggctctatggGGAGGCTATCCAGCG
TGAGTCTCTCCTACCCTCCCGCTCTGGTCCTTCCTCTCCCGCTCTGCACC
CTCTGTGGCCCTCGCTGTGCTCTCTCGCTCCGTGACTTCCCTTCTCCAAG
TTCTCCTTGGTGGCCCGCCGTGGGGCTAGTCCAGGGCTGGATCTCGGGGA
AGCGGCGGGGTGGCCTGGGAGTGGGGAAGGGGGTGCGCACCCGGGACGCG
CGCTACTTGCCCCTTTCGGCGGGGAGCAGGGGAGACCTTTGGCCTACGGC
GACGGGAGGGTCGGGACAAAGTTTAGGGCGTCGATAAGCGTCAGAGCGCC
GAGGTTGGGGGAGGGTTTCTCTTCCGCTCTTTCGCGGGGCCTCTGGCTCC
CCCAGCGCAGCTGGAGTGGGGGACGGGTAGGCTCGTCCCAAAGGCGCGGC
GCTGAGGTTTGTGAACGCGTGGAGGGGCGCTTGGGGTCTGGGGGAGGCGT
CGCCCGGGTAAGCCTGTCTGCTGCGGCTCTGCTTCCCTTAGACTGGAGAG
CTGTGGACTTCGTCTAGGCGCCCGCTAAGTTCGCATGTCCTAGCACCTCT
GGGTCTATGTGGGGCCACACCGTGGGGAGGAAACAGCACGCGACGTTTGT
AGAATGCTTGGCTGTGATACAAAGCGGTTTCGAATAATTAACTTATTTGT
TCCCATCACATGTCACTTTTAAAAAATTATAAGAACTACCCGTTATTGAC
ATCTTTCTGTGTGCCAAGGACTTTATGTGCTTTGCGTCATTTAATTTTGA
AAACAGTTATCTTCCGCCATAGATAACTACTATGGTTATCTTCTGCCTCT
CACAGATGAAGAAACTAAGGCACCGAGATTTTAAGAAACTTAATTACACA
GGGGATAAATGGCAGCAATCGAGATTGAAGTCAAG

In some embodiments, the scHLA-E trimer transgene flanked by homology arms is flanked by sgRNA targeting sequences for the desired locus, so that once co-delivered in the cells together to the CRISPR components, the donor nucleic acid sequence is released as linear DNA from the plasmid following cut by CRISPR/Cas. In some embodiments, the scHLA-E trimer transgene flanked by homology arms is flanked by the sequence of the gRNA pair that target the B2M locus. The resulting sequence is referred to as scHLA-E_trimer_B2M-900HAs_CTS (SEQ ID NO: 4):

CCGTGGCCTTAGCTGTGCTCGCGCTACTCTCTCTTTCTGGCCTGGAGGTA
ATTCATTCATTCATCCATCCATTCGTTCATTCGGTTTACTGAGTACCTAC
TATGTGCCAGCCCCTGTTCTAGGGTGGAAACTAAGAGAATGATGTACCTA
GAGGGCGCTGGAAGCTCTAAAGCCCTAGCAGTTACTGCTTTTACTATTAG
TGGTCGTTTTTTTCTCCCCCCCGCCCCCCGACAAATCAACAGAACAAAGA
AAATTACCTAAACAGCAAGGACATAGGGAGGAACTTCTTGGCACAGAACT
TTCCAAACACTTTTTCCTGAAGGGATACAAGAAGCAAGAAAGGTACTCTT
TCACTAGGACCTTCTCTGAGCTGTCCTCAGGATGCTTTTGGGACTATTTT
TCTTACCCAGAGAATGGAGAAACCCTGCAGGGAATTCCCAAGCTGTAGTT
ATAAACAGAAGTTCTCCTTCTGCTAGGTAGCATTCAAAGATCTTAATCTT
CTGGGTTTCCGTTTTCTCGAATGAAAAATGCAGGTCCGAGCAGTTAACTG
GCTGGGGCACCATTAGCAAGTCACTTAGCATCTCTGGGGCCAGTCTGCAA
AGCGAGGGGGCAGCCTTAATGTGCCTCCAGCCTGAAGTCCTAGAATGAGC
GCCCGGTGTCCCAAGCTGGGGCGCGCACCCCAGATCGGAGGGCGCCGATG
TACAGACAGCAAACTCACCCAGTCTAGTGCATGCCTTCTTAAACATCACG
AGACTCTAAGAAAAGGAAACTGAAAACGGGAAAGTCCCTCTCTCTAACCT
GGCACTGCGTCGCTGGCTTGGAGACAGGTGACGGTCCCTGCGGGCCTTGT
CCTGATTGGCTGGGCACGCGTTTAATATAAGTGGAGGCGTCGCGCTGGCG
GGCATTCCTGAAGCTGACAGCATTCGGGACGAGATGTCTCGCTCAGTCGC
CTTAGCTGTGCTCGCGCTACTCTCTCTGTCCGGGCTCGAAGCTGTTATGG
CTCCGCGGACTTTAATTTTAGGTGGTGGCGGATCCGGTGGTGGCGGTTCT
GGTGGTGGCGGCTCCATCCAGCGTACGCCAAAGATTCAGGTTTACTCACG
TCATCCAGCAGAGAATGGAAAGTCAAATTTCCTGAATTGCTATGTGTCTG
GGTTTCATCCATCCGACATTGAAGTTGACTTACTGAAGAATGGAGAGAGA
ATTGAAAAAGTGGAGCATTCAGACTTGTCTTTCAGCAAGGACTGGTCTTT
CTATCTCTTGTACTACACTGAATTCACCCCCACTGAAAAAGATGAGTATG
CCTGCCGTGTGAACCATGTGACTTTGTCACAGCCCAAGATAGTTAAGTGG
GATCGCGACATGGGTGGTGGCGGTTCTGGTGGTGGCGGTAGTGGCGGCGG
AGGAAGCGGTGGTGGCGGTTCCGGATCTCACTCCTTGAAGTATTTCCACA
CTTCCGTGTCCCGGCCCGGCCGCGGGGAGCCCCGCTTCATCTCTGTGGGC
TACGTGGACGACACCCAGTTCGTGCGCTTCGACAACGACGCCGCGAGTCC
GAGGATGGTGCCGCGGGCGCCGTGGATGGAGCAGGAGGGGTCAGAGTATT
GGGACCGGGAGACACGGAGCGCCAGGGACACCGCACAGATTTTCCGAGTG
AACCTGCGGACGCTGCGCGGCTACTACAATCAGAGCGAGGCCGGGTCTCA
CACCCTGCAGTGGATGCATGGCTGCGAGCTGGGGCCCGACAGGCGCTTCC
TCCGCGGGTATGAACAGTTCGCCTACGACGGCAAGGATTATCTCACCCTG
AATGAGGACCTGCGCTCCTGGACCGCGGTGGACACGGCGGCTCAGATCTC
CGAGCAAAAGTCAAATGATGCCTCTGAGGCGGAGCACCAGAGAGCCTACC
TGGAAGACACATGCGTGGAGTGGCTCCACAAATACCTGGAGAAGGGGAAG
GAGACGCTGCTTCACCTGGAGCCCCCAAAGACACACGTGACTCACCACCC
CATCTCTGACCATGAGGCCACCCTGAGGTGCTGGGCTCTGGGCTTCTACC
CTGCGGAGATCACACTGACCTGGCAGCAGGATGGGGAGGGCCATACCCAG
GACACGGAGCTCGTGGAGACCAGGCCTGCAGGGGATGGAACCTTCCAGAA
GTGGGCAGCTGTGGTGGTGCCTTCTGGAGAGGAGCAGAGATACACGTGCC
ATGTGCAGCATGAGGGGCTACCCGAGCCCGTCACCCTGAGATGGAAGCCG
GCTTCCCAGCCCACCATCCCCATCGTGGGCATCATTGCTGGCCTGGTTCT
CCTTGGATCTGTGGTCTCTGGAGCTGTGGTTGCTGCTGTGATATGGAGGA
AGAAGAGCTCAGGTGGAAAAGGAGGGAGCTACTATAAGGCTGAGTGGAGC
GACAGTGCCCAGGGGTCTGAGTCTCACAGCTTGTAAtaaacccgctgatc
agcctcgactgtgccttctagttgccagccatctgttgtttgcccctccc
ccgtgccttccttgaccctggaaggtgccactcccactgtcctttcctaa
taaaatgaggaaattgcatcgcattgtctgagtaggtgtcattctattct
ggggggtggggggggcaggacagcaagggggaggattgggaagacaatag
caggcatgctggggatgcggtgggctctatggGGAGGCTATCCAGCGTGA
GTCTCTCCTACCCTCCCGCTCTGGTCCTTCCTCTCCCGCTCTGCACCCTC
TGTGGCCCTCGCTGTGCTCTCTCGCTCCGTGACTTCCCTTCTCCAAGTTC
TCCTTGGTGGCCCGCCGTGGGGCTAGTCCAGGGCTGGATCTCGGGGAAGC
GGCGGGGTGGCCTGGGAGTGGGGAAGGGGGTGCGCACCCGGGACGCGCGC
TACTTGCCCCTTTCGGCGGGGAGCAGGGGAGACCTTTGGCCTACGGCGAC
GGGAGGGTCGGGACAAAGTTTAGGGCGTCGATAAGCGTCAGAGCGCCGAG
GTTGGGGGAGGGTTTCTCTTCCGCTCTTTCGCGGGGCCTCTGGCTCCCCC
AGCGCAGCTGGAGTGGGGGACGGGTAGGCTCGTCCCAAAGGCGCGGCGCT
GAGGTTTGTGAACGCGTGGAGGGGCGCTTGGGGTCTGGGGGAGGCGTCGC
CCGGGTAAGCCTGTCTGCTGCGGCTCTGCTTCCCTTAGACTGGAGAGCTG
TGGACTTCGTCTAGGCGCCCGCTAAGTTCGCATGTCCTAGCACCTCTGGG
TCTATGTGGGGCCACACCGTGGGGAGGAAACAGCACGCGACGTTTGTAGA
ATGCTTGGCTGTGATACAAAGCGGTTTCGAATAATTAACTTATTTGTTCC
CATCACATGTCACTTTTAAAAAATTATAAGAACTACCCGTTATTGACATC
TTTCTGTGTGCCAAGGACTTTATGTGCTTTGCGTCATTTAATTTTGAAAA
CAGTTATCTTCCGCCATAGATAACTACTATGGTTATCTTCTGCCTCTCAC
AGATGAAGAAACTAAGGCACCGAGATTTTAAGAAACTTAATTACACAGGG
GATAAATGGCAGCAATCGAGATTGAAGTCAAGCCGTGGCCTTAGCTGTGC
TCGCGCTACTCTCTCTTTCTGGCCTGGAGG

Graft rejection due to alloreactivity is a complication associated to the use of donor-derived allogeneic cells/tissue. HLA proteins are antigen-presenting receptors present on the cell membrane that interact with the T-cell receptor (TCR) to mediate immunosurveillance by the adaptive immune system.

Class 1 HLA proteins encoded at the major histocompatibility 1 (MHC-1) locus (HLA-A/-B/-C/-E/-F/-G) form heterodimeric receptors with beta2-microglobulin (B2M) and present intracellular antigens at the surface of most cells. In the case of an allogeneic graft, the antigens presented by HLA class 1 proteins are recognized as foreign by the host CD8+ cytotoxic T cell via the TCR complex leading to the direct cytolytic attack and loss of the infused cells. In addition to recognizing antigens presented by HLA receptors, TCRs also directly engage and recognize HLA receptors themselves, identifying them as either “self” or “non-self”.

Class 2 HLAs encoded at the MHC-2 locus (HLA-DR/-DQ/-DP) form heterodimers composed of alpha and beta chains that present extracellular antigens and are constitutively expressed by specialized antigen-presenting cells such as macrophages and dendritic cells, and by other cell types including microglia, endothelial, and epithelial cells in response to inflammatory cytokines. Foreign extracellular antigens presented by class 2 HLAs activate the CD4/TCR-mediated response of CD4+ helper T cells that recruit cytotoxic T cells and NK cells through secreted chemokines. Recognition of allogeneic grafts as nonself, either through mismatched HLA proteins or through foreign antigen presentation, leads to host T cell alloreactivity and consequent rejection of the graft through inflammation and cytolytic attack.

HLA matching is, therefore, essential for successful cell and tissue grafting. However, the genes encoding HLA-A, HLA-B, HLA-C, and HLA-DQ are some of the most highly polymorphic coding loci in the human population, therefore HLA matching of allografts is a major challenge to overcome for both conventional cell and tissue donation and for the application of iPSC-derived cellular therapeutics.

Owing to the dependence of HLA class 1 proteins on dimerization with B2M for presentation on the cell surface, genetic modification of the B2M locus has the potential to render allogeneic cells invisible to cytotoxic CD8+ T cells and evade elimination by the patient's immune system. Similarly, disrupting the function of the class 2 transactivator protein (CIITA) switches off the expression of HLA class 2 proteins, rendering cells invisible to CD4+ helper T cells. However, although such approaches evade T cell-mediated immunosurveillance, complete erasure of HLA expression elicits an NK cell-mediated “missing-self” response. This ‘missing self’ response can be prevented by forced expression of minimally polymorphic HLA-E molecules. To obtain inducible, regulated, surface expression of HLA-E without surface expression of HLA-A, B or C, a single-chain HLA-E trimer (scHLA-E trimer) comprising HLA-E, B2M and an antigen peptide can be knocked-in in the B2M locus. Without wishing to be bound by this theory, this approach (depletion of B2M and forced expression of HLA-E) will confer resistance to NK-mediated killing, while the cells will not be recognized as foreign by the host CD8+ T cells.

To prove the simultaneous knock-in knock-out strategy in iPSC, the present disclosure generates base editing knock-out of CIITA gene and simultaneous knock-in of the scHLA-E trimer sequence in the B2M locus with B2M gene knock-out.

A common method for locus specific integration is using CRISPR-Cas technologies to target and cleave the site of interest in the presence of an exogenous DNA sequence that has complementary regions to the genome and the region that is desired to be altered. The DNA sequence for insertion by CRISPR-Cas technology can be supplied by multiple methods, including that of transduction with non-integrating adeno-associated virus (AAV) or supplying a DNA template. The exogenous template for insertion into the genome by CRISPR-Cas requires the insert sequence to be flanked by specific regions being cleaved by the CRISPR-Cas technology, which are commonly known as “homology-arms”.

In some embodiments, exogenous DNA templates or exogenous sequences are supplied as single-stranded and double-stranded DNAs. The DNA can then be either in an open linear structure where the 5′ and 3′ ends of the DNA are exposed or can be a closed where there are no exposed ends of the DNA. The closed DNA molecules includes, but are not limited to, a circular dsDNA, a linear dsDNA, plasmids, minicircles, circularised ssDNA, and doggybone DNA (dbDNA). In some embodiments, the exogenous sequence is delivered as a linear dsDNA.

In some embodiments, the exogenous sequence is delivered in a circular dsDNA. In some embodiments, if a circular dsDNA is used for the delivery of the exogenous sequence, the exogenous DNA is flanked by homology arms from the locus to be inserted. In some embodiments, if a circular dsDNA is used for the delivery of the exogenous sequence, the exogenous DNA flanked by homology arms from the locus to be inserted is flanked on both sides by sgRNAs targeting sequences that target the locus to be inserted.

In some embodiments, the exogenous sequence is introduced into the cell using a viral vector. In some embodiments, the viral vector is selected from the group consisting of lentivirus, retrovirus, adeno-associated virus (AAV) and adenovirus. AAVs have different serotypes, which have different preferences for tissue types. Examples of AAV that can be used in the present disclosure include, but are not limited to, AAV1, AAV2, AAV5, AAV6, AAV8, AAV9, AAV-DJ, AAV-DJ9. In some embodiments, the viral vector is AAV serotype 6 (AAV6). AAV6 vectors have been shown to result in improved transduction efficiency in the field of CAR-T cell generation (Wang et al, Nucleic Acid Research 2016, the contents of which are incorporated herein by reference). AAV is a small icosahedral non-enveloped virus of 25 nm in diameter containing a single stranded DNA genome and in recent years has become an essential therapeutic gene delivery tool. It is used frequently to deliver genetic material into target cells in vivo to treat disease and has been used extensively in clinical application in academia and industry (Hamieh, M et al, Nature 2019. 568 (7750) 112-116, the contents of which are incorporated herein by reference).

In an embodiment, the method of exogenous gene delivery of the exogenous sequence (e.g., CAR, TCR or scHLA-E) sequence into the cell is by AAV6. In another embodiment, the method of exogenous gene delivery of a CAR or TCR or scHLA-E sequence into the cell is by using a plasmid or linear DNA. The site of integration in a specific locus will disrupt the endogenous gene (e.g., TRAC, B2M or CISH), whilst inserting an exogenous DNA fragment that comprises a transgene (e.g., CAR, TCR or scHLA-E genes). In some embodiments, the exogenous sequence is inserted under the control of the endogenous promoter. In other embodiments, the exogenous sequence is controlled by its own promoter.

In some embodiments, the method or system of the disclosure comprises the integration of more than one exogenous sequence. In some embodiments, multiple CARs exogenous sequences are integrated, these multiple CARs recognize different antigens to generate, for example, dual targeting CAR-T cells to limit antigen escape during treatment.

### RNA Guided Nickase

As used herein, a Cas protein, a CRISPR-associated protein, or a CRISPR protein, used interchangeably, refers to a protein of or derived from a CRISPR-Cas type I, type II, or type III system, which has an RNA-guided DNA-binding domain. Non-limiting examples of suitable CRISPR/Cas proteins include, but are not limited to, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. Non-limiting examples of RNA guided nickases capable of interacting with the first and second sgRNAs of the CRISPR system and with the RNA scaffold of the base editing system include, but are not limited to, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1(or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966See e.g., Koonin and Makarova, 2019, Origins and Evolution of CRISPR-Cas systems, Review Philos Trans R Soc Lond B Biol Sci. 2019 May 13; 374(1772); for Type-V Yan et al., Science, 363 μg 88-92, 2019; and for Miniature Cas14, Harrington et al., 2018, Science, vol 362 μg 839-842, the contents of which are incorporated herein by reference in their entireties.

The sequence-targeting component of the methods and systems provided herein typically utilizes a Cas protein of CRISPR/Cas systems from bacterial species as the RNA guided nickase. In some embodiments, the Cas protein is from a type II CRISPR system. See, e.g., Makarova, K. S., Wolf, Y. I., Iranzo, J. et al. Evolutionary classification of CRISPR-Cas systems: a burst of class 2 and derived variants. Nat Rev Microbiol 18, 67-83 (2020), the entire content of which is incorporated herein by reference.

In one embodiment, the Cas protein is derived from a type II CRISPR-Cas system. In exemplary embodiments, the Cas protein is or is derived from a Cas9 protein. In some embodiments, the RNA guided nickase is a Cas9 protein, is derived from a Cas9 protein or comprises a mutation compared to the WT Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum the rmopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Legionella pneumophila, Francisella novicida, gamma proteobacterium HTCC5015, Parasutterella excrementihominis, Sutter ella wadsworthensis, Sulfurospirillum sp. SC ADC, Ruminobacter sp. RM87, Burkholderiales bacterium 1 1 47, Bacteroidetes oral taxon 274 str. F0058, Wolinella succinogenes, Burkholderiales bacterium YL45, Ruminobacter amylophilus, Campylobacter sp. P0111, Campylobacter sp. RM9261, Campylobacter lanienae strain RM8001, Camplylobacter lanienae strain P0121, Turicimonas muris, Legionella londiniensis, Salinivibrio sharmensis, Leptospira sp. isolate FW.030, Moritella sp. isolate NORP46, Fndozoicomonassp. S-B4-1 U, Tamilnaduibacter salinus, Vibrio natriegens, Arcobacter skirrowii, Francisella philomiragia, Francisella hispaniensis, or Parendozoicomonas haliclonae.

The Cas protein or the RNA guided nickase may be obtained as a recombinant fusion polypeptide by methods known in the art, e.g., as a fusion protein with glutathione-s-transferase (GST), 6x-His epitope tag, or M13 Gene 3 protein and expressed in a suitable host cell. Alternatively, the Cas protein or the RNA guided nickase can be chemically synthesized (see, e.g., Creighton, “Proteins: Structures and Molecular Principles,” W.H. Freeman & Co., NY, 1983), or produced by recombinant DNA technology as described herein. For additional guidance, skilled artisans may consult Frederick M. Ausubel et al., Current Protocols in Molecular Biology, John Wiley & Sons, 2003; and Sambrook et al., Molecular Cloning, A Laboratory Manual,” Cold Spring Harbor Press, Cold Spring Harbor, NY, 2001,) the entire contents of each are incorporated herein by reference in their entireties.

In another embodiment, the RNA guided nickase is the nuclease defective nickase nCas9 from S. pyogenes D10A mutant (Cas9(D10A)). In another embodiment, the RNA guided nickase is the nuclease defective nickase nCas9 H840A mutant. In another embodiment, the RNA guided nickase is a nuclease protein that only breaks one DNA strand. In some embodiments, the RNA guided nickase comprises the sequence set forth in SEQ ID NO: 19. In some embodiments, the RNA guided nickase consists of the sequence set forth in SEQ ID NO: 19.

Table 1 lists a non-exhausting list of examples of Cas9, and their corresponding PAM requirements. One can also use synthetic Cas substitutes such as those described in Rauch et al., Programmable RNA-Guided RNA Effector Proteins Built from Human Parts. Cell Volume 178, Issue 1, 27 Jun. 2019, Pages 122-134.e12, the entire content of which is incorporated herein by reference. In some embodiments, the RNA guided nickase is a functional variant or fragment of the Cas protein or synthetic Cas substitutes described herein. The functional variant or fragment shares at least about 70% (e.g., at least about 80%, 90%, 95%, 96%, 97%, 98%, 99%) homology with the Cas protein or synthetic Cas substitute.

TABLE 1

| Species | PAM |
|---|---|
| Streptococcus pyogenes | NGG |
| Streptococcus agalactiae | NGG |
| Staphylococcus aureus | NNGRRT |
| Streptococcus thermophiles | NNAGAAW |
| Streptococcus thermophiles | NGGNG |
| Neisseria meningitidis | NNNNGATT |
| Treponema denticola | NAAAAC |
| Other Type II CRISPR/Cas9 systems | |
| from other bacterial species | |

In some aspects of this disclosure, the above-described sequence-targeting component comprises a fusion between (a) a RNA guided nickase, and (b) a uracil DNA glycosylase (UNG) inhibitor peptide (UGI). For example, in some embodiments, the RNA guided nickase comprises a Cas protein, e.g., Cas9 protein, fused to one or more UGI. Such fusion proteins may exhibit an increased nucleic acid editing efficiency as compared to fusion proteins not comprising an UGI domain. In some embodiments, the UGI comprises a wild type UGI sequence or one having the following amino acid sequence: Protein accession number: sp|P14739|UNGI_BPPB2: Uracil-DNA glycosylase inhibitor (UGI). In some embodiments, the UGI peptide comprises the following amino acid sequence:

(SEQ ID NO: 5)
MTNLSDIIEKETGKQLVIQESILMLPEEVEEVIGNKPESDILVHTAYDES
TDENVMLLTSDAPEYKPWALVIQDSNGENKIKML.

In other embodiments, the UGI peptide consists of the sequence set forth in SEQ ID NO: 5.

In some embodiments, the UGI proteins provided herein include fragments of UGI and proteins homologous to a UGI or a UGI fragment. For example, in some embodiments, a UGI comprises a fragment of the amino acid sequence set forth above. In some embodiments, a UGI comprises an amino acid sequence homologous to the amino acid sequence set forth above or an amino acid sequence homologous to a fragment of the amino acid sequence set forth in the UGI sequence above. In some embodiments, proteins comprising UGI or fragments of UGI or homologs of UGI or UGI fragments are referred to as “UGI variants.” A UGI variant shares homology to UGI, or a fragment thereof. For example, a UGI variant comprises at least about 70% sequence identity (e.g., at least about 80%, 90%, 95%, 96%, 97%, 98%, 99%) compared to a wild type UGI, which may be the UGI sequence as set forth above (SEQ ID NO: 5).

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J Biol. Chem. 264:1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J Biol. Chem. 272:21408-21419(1997); Ravishankar et al., X-ray analysis of a complex of Escherichia coli uracil DNA glycosylase (EcUDG) with a proteinaceous inhibitor.

In an embodiment, the RNA guided nickase is the nuclease defective nickase nCas9 from S. pyogenes (D10A mutant) fused to a UGI. In another embodiment, the RNA guided nickase is the nuclease defective nickase nCas9 from S. pyogenes (D10A mutant) fused to two UGI peptides (referred herein to as nCas9-UGI-UGI).

nCas9-UGI-UGI - SEQ ID NO 195:
PKKKRKVDKKYSIGLAIGTNSVGWAVITDEYKVPSKKFKVLGNTDRHSIK
KNLIGALLFDSGETAEATRLKRTARRRYTRRKNRICYLQEIFSNEMAKVD
DSFFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLP
EKYKEIFFDQSKNGYAGYIDGGASQEEFYKFIKPILEKMDGTEELLVKLN
REDLLRKORTFDNGSIPHQIHLGELHAILRRQEDFYPFLKDNREKIEKIL
TFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYD
ENDKLIREVKVITLKSKLVSDFRKDFQFYKVREINNYHHAHDAYLNAVVG
TALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNI
VKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDL
IIKLPKYSLFELENGRKRMLASAGELQKGNELALPSKYVNFLYLASHYEK
LKGSPEDNEQKOLFVEQHKHYLDEIIEQISEFSKRVILADANLDKVLSAY
NKHRDKPIREQAENIIHLFTLTNLGAPAAFKYFDTTIDRKRYTSTKEVLD
ATLIHQSITGLYETRIDLSQLGGDSGGSGGSGGSTNLSDIIEKETGKQLV
IQESILMLPEEVEEVIGNKPESDILVHTAYDESTDENVMLLTSDAPEYKP
WALVIQDSNGENKIKMLSGGSGGSGGSTNLSDIIEKETGKQLVIQESILM
LPEEVEEVIGNKPESDILVHTAYDESTDENVMLLTSDAPEYKPWALVIQD
SNGENKIKMLSGGSKRTADGSEFEPKKKRKV

In some embodiments, the RNA guided nickase capable of interacting with the first and second gRNAs of the CRISPR system and with the RNA scaffold of the base editing system is a single molecule, i.e., the same nickase molecule is interacting with the first and second gRNAs of the CRISPR system and interacting with the RNA scaffold of the base editing system. Therefore, the RNA guided nickase is simultaneously cleaving one strand of a first target nucleic acid sequence and base editing a second/third/fourth target nucleic acid sequence.

gRNA

The CRISPR-Cas system has been used to perform genome-editing in cells of various organisms. The specificity of this system is dictated by base pairing between a target DNA and a custom-designed guide RNA (gRNA). By engineering and adjusting the base-pairing properties of guide RNAs, one can target any nucleic acid sequences of interest provided that there is a PAM sequence in a target sequence. Therefore, the first target nucleic acid sequence or locus where the exogenous sequence is integrated is any locus flanked by PAM sequences recognized by the first and second gRNAs.

The CRISPR system of the present disclosure can be used to integrate a donor nucleic acid sequence comprising an exogenous DNA sequence into a target nucleic acid sequence of a cell, in particular into an immune cell, a T cell or an iPSC. In some embodiments, the exogenous DNA comprises a CAR, a TCR or a scHLA-E trimer encoding sequence, or may code for a therapeutic protein or correct a point mutation/indel in the genome.

According to one embodiment of the disclosure, the first target nucleic acid sequence represents the CAR integration site into the host cell, in particular, into a T cell or iPSC. As described above, the CAR gene to be integrated into the target nucleic acid sequence of the cell may be delivered into the host cell using a viral vector. In some embodiments, CAR gene to be integrated into the target nucleic acid sequence of the cell may be delivered into the host cell using an AAV. In some embodiments, the AAV is AAV6.

According to one embodiment of the disclosure, the first target nucleic acid sequence represents the TCR integration site into the host cell, in particular, into a T cell or iPSC. As described above, the CAR gene to be integrated into the target nucleic acid sequence of the cell may be delivered into the host cell using a viral vector. In some embodiments, TCR gene to be integrated into the target nucleic acid sequence of the cell may be delivered into the host cell using an AAV. In some embodiments, the AAV is AAV6.

According to one embodiment of the disclosure, the first target nucleic acid sequence represents the scHLA-E trimer integration site into the host cell, in particular, into a T cell or iPSC. As described above, the scHLA-E trimer gene to be integrated into the target nucleic acid sequence of the cell may be delivered into the host cell using a viral vector. In some embodiments, scHLA-E trimer gene to be integrated into the target nucleic acid sequence of the cell may be delivered into the host cell using an AAV. In some embodiments, the AAV is AAV6.

For Type II CRISPR systems, the two components of a gRNA are crRNA and tracrRNA, which together form a CRISPR/Cas based module for sequence targeting and recognition. crRNA provides targeting specificity and includes a region that is complementary and capable of hybridization to a pre-selected target site of interest (the guide RNA sequence). tracrRNA is the region of the gRNA that interacts with the Cas protein.

In various embodiments, the crRNA comprises from about 10 nucleotides to more than about 25 nucleotides. In some embodiments, the region of base pairing between the guide sequence and the corresponding target site sequence (crRNA) is about 10, 11, 12, 13, 14, 15, 16, 17, 18, 19, 20, 22, 23, 24, 25, or more than 25 nucleotides in length. In an exemplary embodiment, the crRNA is about 17-20 nucleotides in length, such as 20 nucleotides.

The tracrRNA component of the gRNA specifically binds to the Cas protein and guides the Cas protein to the target DNA or RNA sequence. In some embodiments, the tracrRNA is from Strep pyogenes. In some embodiments, the tracrRNA comprises from about 10 nucleotides to about 50 nucleotides. In some embodiments, tracrRNA is about 10, 11, 12, 13, 14, 15, 16, 17, 18, 19, 20, 22, 23, 24, 25, 30, 35, 40, 45 or more than 50 nucleotides in length.

One requirement for selecting a suitable target nucleic acid is that it has a 3′ protospacer adjacent motif (PAM) site/sequence. Each target sequence and its corresponding PAM site/sequence are referred to herein as a Cas-targeted site. The Type II CRISPR system, one of the most well characterized systems, needs only Cas9 protein and a crRNA complementary to a target sequence to affect target cleavage. As an example, the type II CRISPR system of S. pyogenes uses target sites having N12-20NGG, where NGG represents the PAM site from S. pyogenes, and N12-20 represents the 12-20 nucleotides directly 5′ to the PAM site. Examples of other PAM site sequences from other species of bacteria include, but are not limited, NGGNG, NNNNGATT, NNAGAA, NNAGAAW, and NAAAAC. See, e.g., US 20140273233, WO 2013176772, Cong et al., (2012), Science 339 (6121): 819-823, Jinek et al., (2012), Science 337 (6096): 816-821, Mali et al., (2013), Science 339 (6121): 823-826, Gasiunas et al., (2012), Proc Natl Acad Sci USA. 109 (39): E2579-E2586, Cho et al., (2013) Nature Biotechnology 31, 230-232, Hou et al., Proc Natl Acad Sci USA. 2013 Sep. 24; 110(39):15644-9, Mojica et al., Microbiology. 2009 March; 155(Pt 3):733-40, and www.addgene.org/CRISPR/. The contents of these documents are incorporated herein by reference in their entireties.

In one embodiment, when two or more nickase cleavage sites are required, the PAM sites are designed such that they are 20 to 200 bp apart. In other embodiments, when two or more nickase cleavage sites are required, the PAM sites are designed such that they are 40 and 70 bp apart. In some embodiments, when two or more nickase cleavage sites are required, the PAM sites are outwards (known as a “PAM-out” configuration).

In some embodiments, the target nucleic acid is ssDNA, dsDNA, ssRNA or dsRNA. In some embodiments, the target nucleic acid is either of the two strands on a double stranded nucleic acid in a host cell. In some embodiments, the target nucleic acid is a single stranded nucleic acid. Examples of target nucleic acids include, but are not necessarily limited to, genomic DNA, a host cell chromosome, mitochondrial DNA or a stably maintained plasmid. However, it is to be understood that the present method can be practiced on other target nucleic acids present in a host cell, such as non-stable plasmid DNA, viral DNA, and phagemid DNA, as long as there is a Cas-targeted site, regardless of the nature of the host cell dsDNA. The present method can be practiced on RNAs too.

In some embodiments, the gRNA is a hybrid RNA molecule where the above-described crRNA is fused to a tracrRNA to mimic the natural crRNA:tracrRNA duplex. As used herein, an active portion of a tracrRNA retains the ability to form a complex with a Cas protein, such as Cas9 or dCas9 or nCas9. See, e.g., WO2014144592. Methods for generating crRNA-tracrRNA hybrid RNAs (also known as single guide RNAs or sgRNAs) are known in the art. In embodiments where the crRNA and tracrRNA are provided as a single gRNA (sgRNA), the two components are typically linked together via a tetra loop (also called repeat: anti-repeat). See, e.g., WO2014099750, US 20140179006, and US 20140273226. The contents of these documents are incorporated herein by reference in their entireties.

In some embodiments, the gRNA used in the methods herein may be introduced into the cell as a chemically synthesised RNA molecule. The gRNA may comprise one or more modifications as described herein below. For example, the guide RNA sequence may be chemically modified to include a 2′-(9-methyl phosphorthioate) modification on at least one 5′ nucleotide and/or at least one 3′ nucleotide of the guide RNA sequence. The gRNA may be synthesized as a single molecule (sgRNA), or synthesized or expressed as two separate components, optionally, wherein the first component comprises (a) the crRNA and the second component comprises (b) the tracrRNA. The two components may then be allowed to hybridize prior to introduction into the cell.

In accordance with the first aspect of the disclosure, the method employs a first gRNA and a second gRNA. These function simultaneously to guide the RNA guided nickase to opposite strands of the target DNA site to effect a (staggered) DSB. Therefore, design of the first and second gRNA dictates the precise location for the DSB. The location of the DSB and the design of the homology arms of the donor nucleic acid influence the precise location of the integration of the donor sequence.

The disclosure also provides a method for making multiple genetic modifications to a cell, the method comprising introducing into the cell and/or expressing in the cell: a) a CRISPR system for integrating an exogenous sequence at a first target nucleic acid sequence, the CRISPR system comprising a first sgRNA and a second sgRNA that are complementary to opposite strands of the first target nucleic acid sequence; and a donor nucleic acid sequence comprising the exogenous sequence; and a base editing system for introducing a genetic modification at a second target nucleic acid sequence, the base editing system comprising: an RNA scaffold comprising a guide RNA sequence that is complementary to the second target nucleic acid sequence and, a recruiting RNA motif; and an effector fusion protein comprising an RNA binding domain capable of binding to the recruiting RNA motif and an effector domain comprising a base modifying enzyme; and a single RNA guided nickase capable of interacting with the first and second sgRNAs of the CRISPR system and the RNA scaffold of the base editing system; and culturing the cell to produce a cell comprising multiple genetic modifications.

In one embodiment, the first gRNA and second gRNA bind opposite strands of the first target nucleic acid sequence.

In an embodiment, the exogenous sequence is integrated at the TRAC locus. In another embodiment, the CAR encoding sequence is integrated at the TRAC locus. In an embodiment, the exogenous sequence is integrated at the B2M locus. In another embodiment, the CAR encoding sequence is integrated at the B2M locus. In an embodiment, the exogenous sequence is integrated at the PDCD1 locus. In another embodiment, the CAR encoding sequence is integrated at the PDCD1 locus. In an embodiment, the exogenous sequence is integrated at the TRBC2 locus. In another embodiment, the CAR encoding sequence is integrated at the TRBC2 locus. In an embodiment, the exogenous sequence is integrated at the TRBC1 locus. In another embodiment, the CAR encoding sequence is integrated at the TRBC1 locus. In an embodiment, the exogenous sequence is integrated at the TRBC1/2 locus. In another embodiment, the CAR encoding sequence is integrated at the TRBC1/2 locus. In an embodiment, the exogenous sequence is integrated at the CD52 locus. In another embodiment, the CAR encoding sequence is integrated at the CD52 locus. In an embodiment, the exogenous sequence is integrated at the CISH locus. In another embodiment, the CAR encoding sequence is integrated at the CISH locus.

In another embodiment, the exogenous sequence expression is driven by the endogenous promoter of the locus where it is integrated. In another embodiment, the exogenous sequence expression is driven by its own promoter. In another embodiment, CAR expression is driven by the TRAC endogenous promoter. In another embodiment, CAR expression is driven by its own promoter.

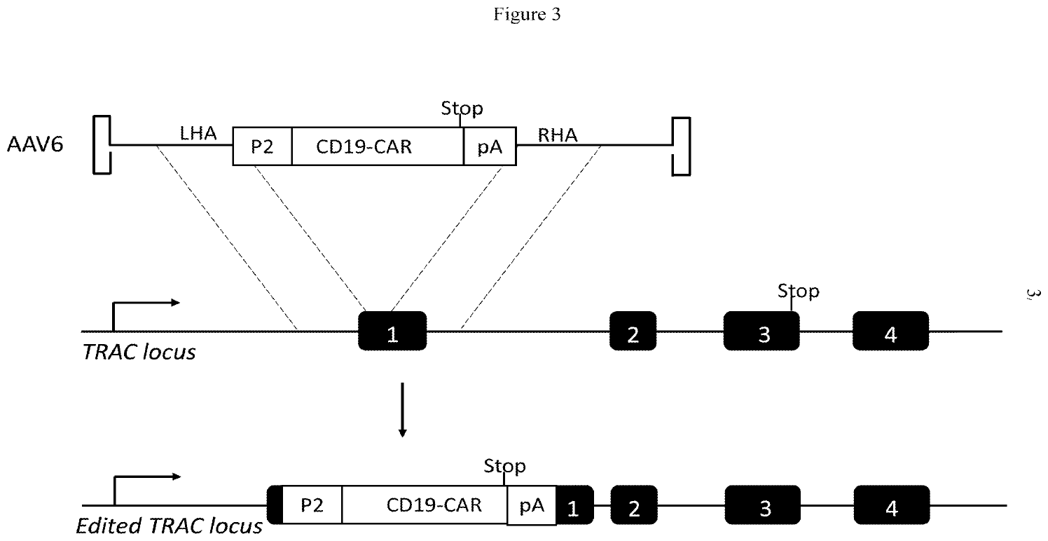

FIG. 3 shows a schematic diagram of an example of a suitable CD19 CAR delivery strategy. In this example, the CAR encoding sequence was inserted in exon 1 of the TRAC locus in frame with the upstream TRAC locus and before the transmembrane domain of the T cell receptor alpha chain variable region. The CAR encoding sequence in the AAV6 vector was flanked by left homology arm (LHA) and right homology arm (RHA) to the sequence left and right to the double nicks in the TRAC exon 1. A 2A peptide derived from porcine teschovirus-1 (P2A) was introduced to avoid interference from the T cell receptor alpha chain variable region. Typically, 2A peptides have approximately 20 amino acids and “self cleavage” occurs between the last two amino acids, glycine (G) and proline (P).

The present disclosure may further comprise additional modular components comprising multiple base editing systems. If more than one module is used, then each guide RNA sequence is complementary to a unique sequence allowing editing of more than one nucleic acid site. The modular system provides the tools for the targeting of multiple loci (e.g., 2 to 10) such that more than one gene can be knocked out simultaneously or sequentially.

Therefore, the method of the disclosure can use a modular system, wherein each module comprises a base editing system for introducing a genetic modification at a second or further target nucleic acid sequence, i.e., multiple base editing systems capable of binding to different target nucleic acid sequences to genetically modify multiple different genetic loci. Each module present in the modular system comprises:
a) an RNA scaffold comprising
   i) a guide RNA sequence complementary to the second or further target nucleic acid sequence,
   ii) a recruiting RNA motif, and
b) an effector fusion protein comprising
   i) an RNA binding domain capable of binding to the recruiting RNA motif,

The second or further target nucleic acid sequences are distinct from the first target nucleic acid sequence.

The RNA scaffold may additionally comprise a tracrRNA that is capable of binding to the RNA guided nickase.

The data provided herein compares the system of the present disclosure with another base editing system (referred to herein as the alternative fusion Cytidine Base Editing (CBE) system), which employs direct fusion of a Cas protein to an effector protein (e.g., a deaminase). The modular design of the system of the present disclosure allows for flexible system engineering. Modules are interchangeable and many combinations of different modules can be achieved by simply swapping the nucleotide sequence of the RNA scaffold. Recruitment of an effector by direct fusion or direct interaction with the protein component of the sequence-targeting unit, on the other hand, always requires a re-engineering of a new fusion protein, which is technically more difficult with a less predictable outcome. The system described herein is based on the base editing (BE) method which was developed to exploit the DNA targeting ability of Cas9 devoid of double strand cleavage activity, combined with the DNA editing capabilities of a deaminase, such as APOBEC-1, an enzyme member of the APOBEC family of DNA/RNA cytidine deaminases. By directly fusing the deaminase effector to a Cas protein that is devoid of double strand cleavage activity, e.g., dCas9 or nCas9 protein, these tools, called base editors, can introduce targeted point mutations in genomic DNA or RNA without generating DSBs or requiring HDR activity. In essence, the BE system utilizes a CRISPR/Cas9 complex devoid of double strand cleavage activity as a DNA targeting machinery, in which the mutant Cas9 serves as an anchor to recruit cytidine or adenine deaminase through a direct protein-protein fusion. As previously described in GB2015204.7 and GB2010692.8 (the entire contents of which are incorporated herein by reference), and used herein, the RNA component (scaffold) of the CRISPR/Cas9 complex serves as an anchor for effector recruitment by including an RNA motif (aptamer) into the RNA molecule. The RNA aptamer recruits an effector, e.g., a base editing enzyme, fused to the RNA aptamer ligand.

The methods provided herein may target different genes in the immune cells to, for example, introduce a genetic modification that results in the desired base change and/or subsequent phenotypic loss of a protein. Examples of genes that can be targeted include, but are not limited to, any of the following genes TRAC, TRBC1, TRBC2, PDCD1, CD52, CISH, CIITA, and B2M. The methods may be used to edit one or both alleles of a target gene in a cell. The methods provided herein may be used to edit multiple different genes (multiplex base editing), and successfully edit one or both alleles of the target genes. For example, the method may use multiple RNA scaffolds comprising different guide RNA sequences to genetically modify (base edit) multiple different genetic loci, (e.g., 2 to 10). Advantageously, it has been shown herein that the system can be used to base edit multiple genes to produce functional knock-outs, for example, by the introduction of point mutations to one or both alleles of the target genes, and at the same time, introducing an exogenous sequence in the cell.

### RNA Scaffold

The RNA scaffold can be either a single RNA molecule or be part of a complex of multiple RNA molecules. For example, the crRNA, the optional tracrRNA, and recruiting RNA motif can be three segments of one, long single RNA molecule. Alternatively, one, two or three of them can be on separate molecules. In the latter case, the three components can be linked together to form the scaffold via covalent or non-covalent linkage or binding, including, e.g., Watson-Crick base-pairing.

In one embodiment, the RNA scaffold comprises two separate RNA molecules. The first RNA molecule can comprise the programmable crRNA and a region that can form a stem duplex structure with a complementary region. The second RNA molecule can comprise the complementary region in addition to the tracrRNA and the RNA motif. Via this stem duplex structure, the first and second RNA molecules form an RNA scaffold of this disclosure. In one embodiment, the first and second RNA molecules each comprise a sequence (of about 6 to about 20 nucleotides) that base pairs to the other sequence. In some embodiments, the tracrRNA and the RNA motif can also be on different RNA molecule and be brought together with another stem duplex structure. In some embodiments, the crRNA and the tracrRNA are part of a single RNA molecule.

The RNAs and related scaffolds of this disclosure can be made by various methods known in the art including cell-based expression, in vitro transcription, and chemical synthesis. The ability to chemically synthesize relatively long RNAs (as long as 200 mers or more) using TC-RNA chemistry (see, e.g., U.S. Pat. No. 8,202,983) allows one to produce RNAs with special features that outperform those enabled by the basic four ribonucleotides (A, C, G, and U). The Cas protein-guide RNA scaffold complexes can be made with recombinant technology using a host cell system or an in vitro translation-transcription system known in the art. Details of such systems and technology can be found in e.g., WO2014144761 WO2014144592, WO2013176772, US20140273226, and US20140273233, the contents of which are incorporated herein by reference in their entireties. The complexes can be isolated or purified, at least to some extent, from cellular material of a cell or an in vitro translation-transcription system in which they are produced.

### RNA Motif

The base editing systems and methods provided herein are based on RNA scaffold-mediated effector protein recruitment. More specifically, the platform takes advantage of various recruiting RNA motif/RNA binding protein binding pairs. To this end, an RNA scaffold is designed such that an RNA motif (e.g., MS2 operator motif), which specifically binds to an RNA binding protein (e.g., MS2 coat protein, MCP), is linked to the gRNA-CRISPR scaffold. As a result, this RNA scaffold component of the platform disclosed herein is a designed RNA molecule, which comprises a guide RNA sequence, comprising the crRNA for specific DNA/RNA sequence recognition, and optionally a CRISPR RNA motif (tracrRNA) for Cas protein binding. The RNA scaffold component also comprises the RNA motif for effector recruitment (also referred to as the recruiting RNA motif). In this way effector protein fusions can be recruited to the site through their ability to bind to the RNA motif. A non-exhaustive list of examples of recruiting RNA motif/RNA binding protein pairs that could be used in the methods and systems provided herein is summarized in Table 2. In some embodiments, the recruiting RNA motif is an RNA aptamer and the RNA binding protein is an aptamer binding protein. In some embodiments, the recruiting RNA motif is the MS2 phage operator stem-loop and the RNA binding protein is the MS2 Coat Protein (MCP).

As will be apparent to the skilled person, chemically modified versions and/or or sequence variants of the RNA motif and their binding partners may also be utilized. Further examples of recruiting RNA motif/RNA binding protein pairs can be found in, e.g., Pumpens P, et al.; Intervirology; 2016; 59:74-110, and Tars K. (2020); Biocommunication of Phages, 261-292; the contents of which are incorporated herein by reference in their entireties.

TABLE 2
Examples of recruiting RNA motifs that can be used with this disclosure,
as well as their pairing RNA binding proteins/protein domains.

| | Pairing interacting | SEQ ID | |
|---|---|---|---|
| RNA motif | protein* | NO: | Organism |
| Telomerase Ku | Ku | 6-8 | Yeast |
| binding motif | | | |
| Telomerase Sm7 | Sm7 | 9-10 | Yeast |
| binding motif | | | |
| MS2 phage operator | MS2 Coat Protein (MCP) | 11-12 | Phage |
| stem-loop | | | |
| PP7 phage operator | PP7 coat protein (PCP) | 13-14 | Phage |
| stem-loop | | | |
| SfMu phage Com | Com RNA binding | 15-16 | Phage |
| stem-loop | protein | | |
| Non-natural RNA | Corresponding aptamer | | Artificially |
| aptamer | ligand | | designed |

*Recruited proteins are fused to effector proteins, for examples, see Table 3.

The sequences for the above binding pairs are listed below.

1. Telomerase Ku binding motif / Ku heterodimer
a. Ku binding hairpin
(SEQ ID NO: 6)
5′-UUCUUGUCGUACUUAUAGAUCGCUACGUUAUUUCAAUUUUGAAAAUC
UGAGUCCUGGGAGUGCGGA-3′
b. Ku heterodimer
(SEQ ID NO: 7)
MSGWESYYKTEGDEEAEEEQEENLEASGDYKYSGRDSLIFLVDASKAMFE
SQSEDELTPFDMSIQCIQSVYISKIISSDRDLLAVVFYGTEKDKNSVNFK
NIYVLQELDNPGAKRILELDQFKGQQGQKRFQDMMGHGSDYSLSEVLWVC
ANLFSDVQFKMSHKRIMLFTNEDNPHGNDSAKASRARTKAGDLRDTGIFL
DLMHLKKPGGFDISLFYRDIISIAEDEDLRVHFEESSKLEDLLRKVRAKE
TRKRALSRLKLKLNKDIVISVGIYNLVQKALKPPPIKLYRETNEPVKTKT
RTFNTSTGGLLLPSDTKRSQIYGSRQIILEKEETEELKRFDDPGLMLMGF
KPLVLLKKHHYLRPSLFVYPEESLVIGSSTLFSALLIKCLEKEVAALCRY
TPRRNIPPYFVALVPQEEELDDQKIQVTPPGFQLVFLPFADDKRKMPFTE
KIMATPEQVGKMKAIVEKLRFTYRSDSFENPVLQQHFRNLEALALDLMEP
EQAVDLTLPKVEAMNKRLGSLVDEFKELVYPPDYNPEGKVTKRKHDNEGS
GSKRPKVEYSEEELKTHISKGTLGKFTVPMLKEACRAYGLKSGLKKQELL
EALTKHFQD
(SEQ ID NO: 8)
MVRSGNKAAVVLCMDVGFTMSNSIPGIESPFEQAKKVITMFVQRQVFAEN
KDEIALVLFGTDGTDNPLSGGDQYQNITVHRHLMLPDFDLLEDIESKIQP
GSQQADFLDALIVSMDVIQHETIGKKFEKRHIEIFTDLSSRFSKSQLDII
IHSLKKCDISERHSIHWPCRLTIGSNLSIRIAAYKSILQERVKKTWTVVD
AKTLKKEDIQKETVYCLNDDDETEVLKEDIIQGFRYGSDIVPFSKVDEEQ
MKYKSEGKCFSVLGFCKSSQVQRRFFMGNQVLKVFAARDDEAAAVALSSL
IHALDDLDMVAIVRYAYDKRANPQVGVAFPHIKHNYECLVYVQLPFMEDL
RQYMFSSLKNSKKYAPTEAQLNAVDALIDSMSLAKKDEKTDTLEDLFPTT
KIPNPRFORLFQCLLHRALHPREPLPPIQQHIWNMLNPPAEVTTKSQIPL
SKIKTLFPLIEAKKKDQVTAQEIFQDNHEDGPTAK
2. Telomerase Sm7 biding motif / Sm7 homoheptamer
a. Sm consensus site (single stranded)
(SEQ ID NO: 9)
5′-AAUUUUUGGA-3′
b. Monomeric Sm - like protein (archaea)
(SEQ ID NO: 10)
GSVIDVSSQRVNVQRPLDALGNSLNSPVIIKLKGDREFRGVLKSFDLHMN
LVLNDAEELEDGEVTRRLGTVLIRGDNIVYISP
3. MS2 phage operator stem loop / MS2 coat protein
a. MS2 phage operator stem loop
(SEQ ID NO: 11)
5′- GCGCACAUGAGGAUCACCCAUGUGC -3′
b. MS2 coat protein
(SEQ ID NO: 12)
MASNFTQFVLVDNGGTGDVTVAPSNFANGIAEWISSNSRSQAYKVTCSVR
QSSAQNRKYTIKVEVPKGAWRSYLNMELTIPIFATNSDCELIVKAMQGLL
KDGNPIPSAIAANSGIY
4. PP7 phage operator stem loop / PP7 coat protein
a. PP7 phage operator stem loop
(SEQ ID NO: 13)
5′-aUAAGGAGUUUAUAUGGAAACCCUUA -3′
b. PP7 coat protein (PCP)
(SEQ ID NO: 14)
MSKTIVLSVGEATRTLTEIQSTADRQIFEEKVGPLVGRLRLTASLRQNGA
KTAYRVNLKLDQADVVDCSTSVCGELPKVRYTQVWSHDVTIVANSTEASR
KSLYDLTKSLVATSQVEDLVVNLVPLGR.
5. SfMu Com stem loop / SfMu Com binding protein
a. SfMu Com stem loop
(SEQ ID NO: 15)
5′-CUGAAUGCCUGCGAGCAUC-3′
b. SfMu Com binding protein
(SEQ ID NO: 16)
MKSIRCKNCNKLLFKADSFDHIEIRCPRCKRHIIMLNACEHPTEKHCGKR
EKITHSDETVRY

The RNA motif may be positioned at various positions of the RNA scaffold. In some embodiments, the RNA motif is positioned at the 3′ end of the guide RNA, in particular, at the 3′ end of the tracrRNA (if present), at the tetra loop of the gRNA, at stem loop 2 of the tracrRNA (if present) or at the stem loop 3 of the tracrRNA (if present). In some embodiments, the MS2 aptamer is positioned at the 3′ end of the gRNA. In particular, the MS2 aptamer may be positioned at the 3′ end of the tracrRNA (if present), at the tetra loop of the gRNA, at stem loop 2 of the tracrRNA (if present) and at the stem loop 3 of the tracrRNA (if present). The positioning of the RNA motif (such as the MS2 aptamer) is crucial due to the steric hindrance that can result from the bulky loops. In some embodiments, the MS2 aptamer is at the 3′ end of the gRNA. Advantageously, the positioning of the MS2 aptamer at the 3′ end of the gRNA is therefore reducing steric hindrance with other bulky loops of the RNA scaffold.

In some embodiments, the recruiting RNA motif may be linked to the guide RNA (in particular to the tracrRNA, if present) via a linker sequence. The linker sequence may be 2, 3, 4, 5, 6, 7 or more than 7 nucleotides. Advantageously, the linker sequence provides flexibility to the RNA scaffold. The linker sequence may be a GC rich sequence.

Modifications may be made to the recruiting RNA motif. In a particular embodiment, the modification to the MS2 aptamer is a substitution of the Adenine to 2-aminopurine (2-AP) at position 10. Advantageously, the substitution induces conformational changes resulting in greater affinity.

The nucleic acid-targeting motif or guide RNA sequence comprising a crRNA and optionally a CRISPR RNA motif (tracrRNA), can be provided as a single guide RNA (sgRNA). In some embodiments, the two components (the crRNA and the optional tracrRNA) are linked via a “repeat: anti-repeat” or a “tetra loop.” The repeat: anti-repeat upper stem can be extended to increase the flexibility, proper folding and stability of the loop. The tetra loop can be extended by 2, 3, 4, 5, 6, 7 bp or more than 7 bp at

In some embodiments, the RNA scaffold may have one or more of the above mentioned modifications. The one or more modification can be on the different components of the RNA scaffold, e.g., extension of tetra loop of the sgRNA and extension of the RNA motif, or can be on the same component of the RNA scaffold, e.g., extension of the RNA motif and substitution of the RNA motif's nucleotides. In some embodiments, the modifications may be two or more, three or more, four or more, or five or more nucleotides. In one embodiment, the modification may be the extension of the RNA motif and/or may the substitution of one or more nucleotides.

An example of a recruiting RNA motif, as used herein, is the MS2 aptamer. The MS2 aptamer specifically binds to the MS2 bacteriophage coat protein (MCP). In one embodiment, the MS2 aptamer is a wild-type MS2 aptamer (SEQ ID NO:11), a mutant MS2 aptamer or variants thereof. In another embodiment, the MS2 aptamer comprises a C-5 and/or F-5 mutation. In some embodiments, the MS2 aptamer can be a single-copy (i.e., one MS2 aptamer) or a double-copy (i.e., two MS2 aptamers). In some embodiments, the RNA motif is a single-copy RNA motif. In other embodiments, the RNA motif comprises more than one copy.

### Effector Fusion Protein

The effector fusion protein comprises two components, a RNA binding domain capable of binding to the recruiting RNA motif and an effector domain comprising a base modifying enzyme. In some embodiments, the base modifying enzyme has an activity selected from the group consisting of a cytosine deamination activity, an adenosine deamination activity, a DNA methyl transferase activity and a demethylase activity. The terms cytosine and cytidine are used interchangeably with respect to deaminases and deamination activity, as are the terms adenine and adenosine.

### RNA Binding Domain

In some embodiments, the RNA-binding domain is not the RNA binding domain of a Cas protein (such as Cas9) or its variant (such as dCas9 or nCas9). Examples of suitable RNA-binding domains are listed in Table 2, including the RNA motif-RNA binding pairs. Due to the flexibility of the RNA scaffold mediated recruitment, a functional monomer of RNA binding domains, as well as dimers, tetramers, or oligomers could be formed relatively easily near the target DNA or RNA sequence.

### Effector Domain

The effector domain or effector protein, comprises a base modifying enzyme which has cytidine deaminase activity (e.g., AID, APOBEC1, APOBEC3G) or adenosine deaminase activity (e.g., ADA and tadA) or DNA methyl transferase activity (e.g., Dnmt1 and Dnmt3a) or demethylase activity (e.g., Tet1 and Tet2). In some embodiments, the effector is modified to induce or improve DNA editing activity, for example, in the case of ADA and tadA which require modification to edit DNA. In some embodiments, the base modifying enzyme is a wild type or genetically engineered version of AID, CDA, APOBEC1, APOBEC3A, APOBEC3B, APOBEC3C, APOBEC3D, APOBEC3F, or other APOBEC family enzymes.

In some embodiments, the base modifying enzyme is a cytosine deaminase, such as APOBEC1. In some embodiments, the base modifying enzyme is APOBEC1 and comprises the sequence set forth below in SEQ ID NO:17:

MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLK

The effector domain is linked to an RNA binding domain to create the effector fusion protein. This may be by chemical modification, peptide linkers, chemical linkers, covalent or non-covalent bonds, or protein fusion or by any means known to one skilled in the art. The joining can be permanent or reversible. See, for example, U.S. Pat. Nos. 4,625,014, 5,057,301, and 5,514,363, US Application Nos. 20150182596 and 20100063258, and WO2012142515, the contents of which are incorporated herein in their entirety by reference. In some embodiments, the effector domain is linked to the RNA binding domain by a peptide linker.

In some embodiments, the effector fusion protein can comprise other domains, apart from the RNA binding domain and an effector domain. In certain embodiments, the effector fusion protein can comprise at least one nuclear localization signal (NLS). In general, a NLS comprises a stretch of basic amino acids. Nuclear localization signals are known in the art (see, e.g., Lange et al., J. Biol. Chem., 2007, 282:5101-5105, the entire contents of which are incorporated herein by reference). The NLS can be located at the N-terminus, the C-terminal, or in an internal location of the fusion protein.

In some embodiments, the fusion protein can comprise at least one cell-penetrating domain to facilitate delivery of the protein into a target cell. In one embodiment, the cell-penetrating domain can be a cell-penetrating peptide sequence. Various cell-penetrating peptide sequences are known in the art and examples include that of the HIV-1 TAT protein, TLM of the human HBV, Pep-1, VP22, and a polyarginine peptide sequence.

In still other embodiments, the fusion protein can comprise at least one marker domain. Non-limiting examples of marker domains include fluorescent proteins, purification tags, and epitope tags. In some embodiments, the marker domain can be a fluorescent protein. In other embodiments, the marker domain can be a purification tag and/or an epitope tag. See, e.g., US 20140273233, the entire contents of which are incorporated herein by reference.

In one embodiment, AID is used as the effector domain, and as an example to illustrate how the system works. AID is a cytidine deaminase that can catalyze the reaction of deamination of cytidine in the context of DNA or RNA. When brought to the targeted site, AID changes a C base to a U base. In dividing cells, this could lead to a C to T point mutation. Alternatively, the change of C to U could trigger cellular DNA repair pathways, mainly excision repair pathway, which will remove the mismatching U-G base-pair, and replace it with a T-A, A-T, C-G, or G-C pair. As a result, a point mutation would be generated at the target C-G site. As excision repair pathway is present in most, if not all, somatic cells, recruitment of AID to the target site can correct a C-G base pair to others. In that case, if a C-G base pair is an underlying disease-causing genetic mutation in somatic tissues/cells, the above-described approach can be used to correct the mutation and thereby treat the disease.

In another embodiment, APOBEC is used as the effector domain, and as an example to illustrate how the system works. APOBEC is also a cytidine deaminase that can catalyze the reaction of deamination of cytidine in the context of DNA or RNA.

In another embodiment, an adenosine deaminase was used as the effector domain, and as an example to illustrate how the system works. An adenosine deaminase can catalyze the reaction of deamination of adenosine in the context of DNA or RNA. By the same token, if an underlying disease causing genetic mutation is an A-T base pair at a specific site, one can use the same approach to recruit an adenosine deaminase to the specific site, where adenosine deaminase can correct the A-T base pair to others; see, for example, David Liu—U.S. Ser. No. 10/113,163, the entire contents of which are incorporated herein by reference. Other effector enzymes are expected to generate other types of changes in base-pairing. A non-exhaustive list of examples of base modifying enzymes is detailed in Table 3.

In some embodiments, the effector proteins provided herein can include functional variants, such as fragments of the effector proteins and proteins homologous to the fragments or proteins, for example, as described in Table 3. A functional variant is considered to share homology to an effector protein, or a fragment thereof, for example, of at least about 70% (e.g., at least about 80%, 90%, 95%, 96%, 97%, 98%, 99%), compared to the wild-type effector protein.

TABLE 3
Examples of effector proteins that can be used in this disclosure

| Enzyme type | Genetic change | Effector protein abbreviated |
|---|---|---|
| Cytidine deaminase | C→U/T | AID |
| | | APOBEC1 |
| | | APOBEC3A |
| | | APOBEC3B |
| | | APOBEC3C |
| | | APOBEC3D |
| | | APOBEC3F |
| | | APOBEC3G |
| | | APOBEC3H |
| | | CDA |
| Adenosine deaminase | A→I/G | ADA |
| | | ADAR1 |
| | | ADAR2 |
| | | ADAR3 |
| | | tadA |
| DNA Methyl transferase | C→Met-C | Dnmt1 |
| | | Dnmt3a |
| | | Dnmt3b |
| Demethylase | Met-C→ C | Tet1 |
| | | Tet2 |
| | | TDG |

Effector protein full names:
AID: activation induced cytidine deaminase, a.k.a. AICDA
APOBEC1: apolipoprotein B mRNA editing enzyme, catalytic polypeptide-like 1.
APOBEC3A: apolipoprotein B mRNA editing enzyme, catalytic polypeptide-like 3A
APOBEC3B: apolipoprotein B mRNA editing enzyme, catalytic polypeptide-like 3B
APOBEC3C: apolipoprotein B mRNA editing enzyme, catalytic polypeptide-like 3C
APOBEC3D: apolipoprotein B mRNA editing enzyme, catalytic polypeptide-like 3D
APOBEC3F: apolipoprotein B mRNA editing enzyme, catalytic polypeptide-like 3F
APOBEC3G: apolipoprotein B mRNA editing enzyme, catalytic polypeptide-like 3G
APOBEC3H: apolipoprotein B mRNA editing enzyme, catalytic polypeptide-like 3H
ADA: adenosine deaminase
ADAR1: adenosine deaminase acting on RNA 1
ADAR2: adenosine deaminase acting on RNA 2
ADAR3: adenosine deaminase acting on RNA 3
CDA: Cytidine deaminase
Dnmt1: DNA (cytosine-5-)-methyltransferase 1
Dnmt3a: DNA (cytosine-5-)-methyltransferase 3 alpha
Dnmt3b: DNA (cytosine-5-)-methyltransferase 3 beta
tadA: tRNA adenosine deaminase
Tet1: ten-eleven translocation 1
Tet2: ten-eleven translocation 2
Tdg: thymine DNA glycosylase

The above-described three specific components constitute the technological platform. Each component could be chosen from the list in Tables 1-3 respectively to achieve a specific therapeutic/utility goal.

In one embodiment, an RNA scaffold mediated recruitment system was constructed using (i) Cas9, dCas9 or nCas9 from S. pyogenes as the RNA guided nickase, (ii) an RNA scaffold containing a guide RNA sequence, a tracrRNA, and a recruiting RNA motif comprising a MS2 phage operator stem-loop, and (iii) an effector fusion protein containing a human AID fused to MS2 phage operator stem-loop binding protein (MCP). The sequences for the components are listed below.

S. pyogenes dCas9 protein sequence (SEQ ID NO: 18)
MDKKYSIGLAIGTNSVGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRERMKRIEEGIKE
LGSQILKEHPVENTQLQNEKLYLYYLQNGRDMYVDQELDINRLSDYDVDAIVPQSFLKDDSIDNKVLTRS
DKNRGKSDNVPSEEVVKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKRPLIETNGETGEIV
WDKGRDFATVRKVLSMPQVNIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLD
KVLSAYNKHRDKPIREQAENIIHLFTLTNLGAPAAFKYFDTTIDRKRYTSTKEVLDATLIHQSITGLYETRIDLS
QLGGD
(Residues underlined: D10A, H840A active site mutants)
Cas9 D10A Protein (residues underlined: D10A, SEQ ID NO: 19)(nCas9)
DKKYSIGLAIGTNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASGVDAKAILSARLSK
SRRLENLIAQLPGEKKNGLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPEKYKEIFFDQSKNGYAGYI
DGGASQEEFYKFIKPILEKMDGTEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLTFKEDIQKAQVSGQGDSLH
EHIANLAGSPAIKKGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDHIVPQSFLKDDSIDNKVLTRSD
KNRGKSDNVPSEEVVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIRKRPLIETNGETGEIVW
DKGRDFATVRKVLSMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKOLFVEQHKHYLDEIIEQISEFSKRVILADANLDKVL
SAYNKHRDKPIREQAENIIHLFTLTNLGAPAAFKYFDTTIDRKRYTSTKEVLDATLIHQSITGLYETRIDLSQL
GGD
DNA encoding Cas9 D10A Protein (SEQ ID NO: 20)
GATAAAAAGTATTCTATTGGTTTAGCCATCGGCACTAATTCCGTTGGATGGGCTGTCATAACCGATG
AATACAAAGTACCTTCAAAGAAATTTAAGGTGTTGGGGAACACAGACCGTCATTCGATTAAAAAGA
ATCTTATCGGTGCCCTCCTATTCGATAGTGGCGAAACGGCAGAGGCGACTCGCCTGAAACGAACCG
CTCGGAGAAGGTATACACGTCGCAAGAACCGAATATGTTACTTACAAGAAATTTTTAGCAATGAGAT
GGCCAAAGTTGACGATTCTTTCTTTCACCGTTTGGAAGAGTCCTTCCTTGTCGAAGAGGACAAGAAA
CATGAACGGCACCCCATCTTTGGAAACATAGTAGATGAGGTGGCATATCATGAAAAGTACCCAACG
ATTTATCACCTCAGAAAAAAGCTAGTTGACTCAACTGATAAAGCGGACCTGAGGTTAATCTACTTGG
CTCTTGCCCATATGATAAAGTTCCGTGGGCACTTTCTCATTGAGGGTGATCTAAATCCGGACAACTCG
GATGTCGACAAACTGTTCATCCAGTTAGTACAAACCTATAATCAGTTGTTTGAAGAGAACCCTATAAA
TGCAAGTGGCGTGGATGCGAAGGCTATTCTTAGCGCCCGCCTCTCTAAATCCCGACGGCTAGAAAAC
CTGATCGCACAATTACCCGGAGAGAAGAAAAATGGGTTGTTCGGTAACCTTATAGCGCTCTCACTAG
GCCTGACACCAAATTTTAAGTCGAACTTCGACTTAGCTGAAGATGCCAAATTGCAGCTTAGTAAGGA
CACGTACGATGACGATCTCGACAATCTACTGGCACAAATTGGAGATCAGTATGCGGACTTATTTTTG
GCTGCCAAAAACCTTAGCGATGCAATCCTCCTATCTGACATACTGAGAGTTAATACTGAGATTACCAA
GGCGCCGTTATCCGCTTCAATGATCAAAAGGTACGATGAACATCACCAAGACTTGACACTTCTCAAG
GCCCTAGTCCGTCAGCAACTGCCTGAGAAATATAAGGAAATATTCTTTGATCAGTCGAAAAACGGGT
ACGCAGGTTATATTGACGGCGGAGCGAGTCAAGAGGAATTCTACAAGTTTATCAAACCCATATTAGA
GAAGATGGATGGGACGGAAGAGTTGCTTGTAAAACTCAATCGCGAAGATCTACTGCGAAAGCAGC
GGACTTTCGACAACGGTAGCATTCCACATCAAATCCACTTAGGCGAATTGCATGCTATACTTAGAAG
GCAGGAGGATTTTTATCCGTTCCTCAAAGACAATCGTGAAAAGATTGAGAAAATCCTAACCTTTCGC
ATACCTTACTATGTGGGACCCCTGGCCCGAGGGAACTCTCGGTTCGCATGGATGACAAGAAAGTCC
GAAGAAACGATTACTCCATGGAATTTTGAGGAAGTTGTCGATAAAGGTGCGTCAGCTCAATCGTTCA
TCGAGAGGATGACCAACTTTGACAAGAATTTACCGAACGAAAAAGTATTGCCTAAGCACAGTTTACT
TTACGAGTATTTCACAGTGTACAATGAACTCACGAAAGTTAAGTATGTCACTGAGGGCATGCGTAAA
CCCGCCTTTCTAAGCGGAGAACAGAAGAAAGCAATAGTAGATCTGTTATTCAAGACCAACCGCAAA
GTGACAGTTAAGCAATTGAAAGAGGACTACTTTAAGAAAATTGAATGCTTCGATTCTGTCGAGATCT
CCGGGGTAGAAGATCGATTTAATGCGTCACTTGGTACGTATCATGACCTCCTAAAGATAATTAAAGA
TAAGGACTTCCTGGATAACGAAGAGAATGAAGATATCTTAGAAGATATAGTGTTGACTCTTACCCTC
TTTGAAGATCGGGAAATGATTGAGGAAAGACTAAAAACATACGCTCACCTGTTCGACGATAAGGTT
ATGAAACAGTTAAAGAGGCGTCGCTATACGGGCTGGGGACGATTGTCGCGGAAACTTATCAACGGG
ATAAGAGACAAGCAAAGTGGTAAAACTATTCTCGATTTTCTAAAGAGCGACGGCTTCGCCAATAGG
AACTTTATGCAGCTGATCCATGATGACTCTTTAACCTTCAAAGAGGATATACAAAAGGCACAGGTTTC
CGGACAAGGGGACTCATTGCACGAACATATTGCGAATCTTGCTGGTTCGCCAGCCATCAAAAAGGG
CATACTCCAGACAGTCAAAGTAGTGGATGAGCTAGTTAAGGTCATGGGACGTCACAAACCGGAAAA
CATTGTAATCGAGATGGCACGCGAAAATCAAACGACTCAGAAGGGGCAAAAAAACAGTCGAGAGC
GGATGAAGAGAATAGAAGAGGGTATTAAAGAACTGGGCAGCCAGATCTTAAAGGAGCATCCTGTG
GAAAATACCCAATTGCAGAACGAGAAACTTTACCTCTATTACCTACAAAATGGAAGGGACATGTATG
TTGATCAGGAACTGGACATAAACCGTTTATCTGATTACGACGTCGATCACATTGTACCCCAATCCTTT
TTGAAGGACGATTCAATCGACAATAAAGTGCTTACACGCTCGGATAAGAACCGAGGGAAAAGTGAC
AATGTTCCAAGCGAGGAAGTCGTAAAGAAAATGAAGAACTATTGGCGGCAGCTCCTAAATGCGAAA
CTGATAACGCAAAGAAAGTTCGATAACTTAACTAAAGCTGAGAGGGGGGCTTGTCTGAACTTGAC
AAGGCCGGATTTATTAAACGTCAGCTCGTGGAAACCCGCCAAATCACAAAGCATGTTGCACAGATAC
TAGATTCCCGAATGAATACGAAATACGACGAGAACGATAAGCTGATTCGGGAAGTCAAAGTAATCA
CTTTAAAGTCAAAATTGGTGTCGGACTTCAGAAAGGATTTTCAATTCTATAAAGTTAGGGAGATAAA
TAACTACCACCATGCGCACGACGCTTATCTTAATGCCGTCGTAGGGACCGCACTCATTAAGAAATAC
CCGAAGCTAGAAAGTGAGTTTGTGTATGGTGATTACAAAGTTTATGACGTCCGTAAGATGATCGCG
AAAAGCGAACAGGAGATAGGCAAGGCTACAGCCAAATACTTCTTTTATTCTAACATTATGAATTTCTT
TAAGACGGAAATCACTCTGGCAAACGGAGAGATACGCAAACGACCTTTAATTGAAACCAATGGGGA
GACAGGTGAAATCGTATGGGATAAGGGCCGGGACTTCGCGACGGTGAGAAAAGTTTTGTCCATGCC
CCAAGTCAACATAGTAAAGAAAACTGAGGTGCAGACCGGAGGGTTTTCAAAGGAATCGATTCTTCC
AAAAAGGAATAGTGATAAGCTCATCGCTCGTAAAAAGGACTGGGACCCGAAAAAGTACGGTGGCTT
CGATAGCCCTACAGTTGCCTATTCTGTCCTAGTAGTGGCAAAAGTTGAGAAGGGAAAATCCAAGAA
ACTGAAGTCAGTCAAAGAATTATTGGGGATAACGATTATGGAGCGCTCGTCTTTTGAAAAGAACCCC
ATCGACTTCCTTGAGGCGAAAGGTTACAAGGAAGTAAAAAAGGATCTCATAATTAAACTACCAAAGT
ATAGTCTGTTTGAGTTAGAAAATGGCCGAAAACGGATGTTGGCTAGCGCCGGAGAGCTTCAAAAGG
GGAACGAACTCGCACTACCGTCTAAATACGTGAATTTCCTGTATTTAGCGTCCCATTACGAGAAGTT
GAAAGGTTCACCTGAAGATAACGAACAGAAGCAACTTTTTGTTGAGCAGCACAAACATTATCTCGAC
GAAATCATAGAGCAAATTTCGGAATTCAGTAAGAGAGTCATCCTAGCTGATGCCAATCTGGACAAA
GTATTAAGCGCATACAACAAGCACAGGGATAAACCCATACGTGAGCAGGCGGAAAATATTATCCAT
TTGTTTACTCTTACCAACCTCGGCGCTCCAGCCGCATTCAAGTATTTTGACACAACGATAGATCGCAA
ACGATACACTTCTACCAAGGAGGTGCTAGACGCGACACTGATTCACCAATCCATCACGGGATTATAT
GAAACTCGGATAGATTTGTCACAGCTTGGGGGTGAC
RNA scaffold expression cassette (S. pyogenes), containing a 20-nucleotide programmable
sequence, a tracrRNA motif, and an MS2 phage operator stem loop motif (SEQ ID NO: 21):
N₂₀GTTTTAGAGCTAGAAATAGCAAGTTAAAATAAGGCTAGTCCGTTATCAACTTGAAAAAGTGGCA
CCGAGTCGGTGCGCGCACATGAGGATCACCCATGTGCTTTTTTTG
(N₂₀: programmable sequence: crRNA; Underlined: tracrRNA motif; Bold: recruiting RNA
motif - MS2 motif; Italic: terminator)

The above RNA scaffold containing one MS2 loop (1×MS2). Shown below is an RNA scaffold containing two MS2 loops (2×MS2), where MS2 scaffolds are underlined:

(SEQ ID NO: 22)
N₂₀GTTTTAGAGCTAGAAATAGCAAGTTAAAATAAGGCTAGTCCGTTATCAACTTGAAAAAGTGGCA
CCGAGTCGGTGCgggagcACATGAGGATCACCCATGTgccacgagcgACATGAGGATCACCCATGTcgctc
gtgttcccTTTTTTTCTCCGCT
Effector fusion protein comprising an Effector AID -MCP fusion:
(SEQ ID NO: 23)
MDSLLMNRRKFLYQFKNVRWAKGRRETYLCYVVKRRDSATSFSLDFGYLRNKNGCHVELLFLRYISDWD
LDPGRCYRVTWFTSWSPCYDCARHVADFLRGNPNLSLRIFTARLYFCEDRKAEPEGLRRLHRAGVQIAIM

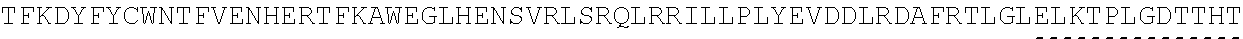

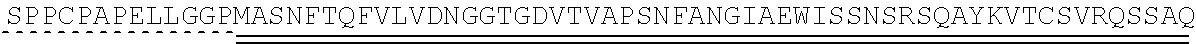

NRKYTIKVEVPKGAWRSYLNMELTIPIFATNSDCELIVKAMQGLLKDGNPIPSAIAANSGIY

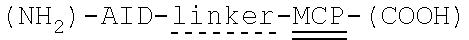

Effector fusion protein comprising an Effector Apobec1-MCP fusion:
(SEQ ID NO: 24)
MSSETGPVAVDPTLRRRIEPHEFEVFFDPRELRKETCLLYEINWGGRHSIWRHTSQNTNKHVEVNFIEKFT
TERYFCPNTRCSITWFLSWSPCGECSRAITEFLSRYPHVTLFIYIARLYHHADPRNRQGLRDLISSGVTIQIM
TEQESGYCWRNFVNYSPSNEAHWPRYPHLWVRLYVLELYCIILGLPPCLNILRRKQPQLTFFTIALQSCHY
QRLPPHILWATGLKELKTPLGDTTHTSPPCPAPELLGGPMASNFTQFVLVDNGGTGDVTVAPSNFANGI
AEWISSNSRSQAYKVTCSVRQSSAQNRKYTIKVEVPKGAWRSYLNMELTIPIFATNSDCELIVKAMQGLL
KDGNPIPSAIAANSGIY

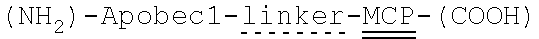

Like the Cas protein described herein, the effector fusion protein can also be obtained as a recombinant polypeptide. Techniques for making recombinant polypeptides are known in the art. See e.g., Creighton, “Proteins: Structures and Molecular Principles,” W.H. Freeman & Co., NY, 1983); Ausubel et al., Current Protocols in Molecular Biology, John Wiley & Sons, 2003; and Sambrook et al., Molecular Cloning, A Laboratory Manual,” Cold Spring Harbor Press, Cold Spring Harbor, NY, 2001, the entire contents of which are incorporated herein by reference).

As described herein, by mutating Ser38 to Ala in AID one can reduce the recruitment of AID to off-target sites. Listed below are the DNA and protein sequences of both wild type AID, as well as AID_S38A (phosphorylation null, pnAID):

wtAID cDNA (Ser38 codon in bold and underlined, SEQ ID NO: 25):
ATGGACAGCCTCTTGATGAACCGGAGGAAGTTTCTTTACCAATTCAAAAATGTCCGCTGGGCTAAGG
GTCGGCGTGAGACCTACCTGTGCTACGTAGTGAAGAGGCGTGACAGTGCTACATCCTTTTCACTGGA
CTTTGGTTATCTTCGCAATAAGAACGGCTGCCACGTGGAATTGCTCTTCCTCCGCTACATCTCGGACT
GGGACCTAGACCCTGGCCGCTGCTACCGCGTCACCTGGTTCACCTCCTGGAGCCCCTGCTACGACTG
TGCCCGACATGTGGCCGACTTTCTGCGAGGGAACCCCAACCTCAGTCTGAGGATCTTCACCGCGCGC
CTCTACTTCTGTGAGGACCGCAAGGCTGAGCCCGAGGGGCTGCGGCGGCTGCACCGCGCCGGGGT
GCAAATAGCCATCATGACCTTCAAAGATTATTTTTACTGCTGGAATACTTTTGTAGAAAACCATGAAA
GAACTTTCAAAGCCTGGGAAGGGCTGCATGAAAATTCAGTTCGTCTCTCCAGACAGCTTCGGCGCAT
CCTTTTGCCCCTGTATGAGGTTGATGACTTACGAGACGCATTTCGTACTTTGGGACTT
wtAID protein (Ser38 in bold and underlined, SEQ ID NO: 26):
MDSLLMNRRKFLYQFKNVRWAKGRRETYLCYVVKRRDSATSFSLDFGYLRNKNGCHVELLFLRYISDWD
LDPGRCYRVTWFTSWSPCYDCARHVADFLRGNPNLSLRIFTARLYFCEDRKAEPEGLRRLHRAGVQIAIM
TFKDYFYCWNTFVENHERTFKAWEGLHENSVRLSRQLRRILLPLYEVDDLRDAFRTLGL
AID_S38A cDNA (S38A mutation in bold and underlined, SEQ ID NO: 27)
ATGGACAGCCTCTTGATGAACCGGAGGAAGTTTCTTTACCAATTCAAAAATGTCCGCTGGGCTAAGG
GTCGGCGTGAGACCTACCTGTGCTACGTAGTGAAGAGGCGTGACGCCGCTACATCCTTTTCACTGGA
CTTTGGTTATCTTCGCAATAAGAACGGCTGCCACGTGGAATTGCTCTTCCTCCGCTACATCTCGGACT
GGGACCTAGACCCTGGCCGCTGCTACCGCGTCACCTGGTTCACCTCCTGGAGCCCCTGCTACGACTG
TGCCCGACATGTGGCCGACTTTCTGCGAGGGAACCCCAACCTCAGTCTGAGGATCTTCACCGCGCGC
CTCTACTTCTGTGAGGACCGCAAGGCTGAGCCCGAGGGGCTGCGGCGGCTGCACCGCGCCGGGGT
GCAAATAGCCATCATGACCTTCAAAGATTATTTTTACTGCTGGAATACTTTTGTAGAAAACCATGAAA
GAACTTTCAAAGCCTGGGAAGGGCTGCATGAAAATTCAGTTCGTCTCTCCAGACAGCTTCGGCGCAT
CCTTTTGCCCCTGTATGAGGTTGATGACTTACGAGACGCATTTCGTACTTTGGGACTT
AID_S38A protein (S38A mutation in bold and underlined, SEQ ID NO: 28)
MDSLLMNRRKFLYQFKNVRWAKGRRETYLCYVVKRRDAATSFSLDFGYLRNKNGCHVELLFLRYISDWD
LDPGRCYRVTWFTSWSPCYDCARHVADFLRGNPNLSLRIFTARLYFCEDRKAEPEGLRRLHRAGVQIAIM
TFKDYFYCWNTFVENHERTFKAWEGLHENSVRLSRQLRRILLPLYEVDDLRDAFRTLGL

The above three components of the platform/system disclosed herein can be expressed using one, two or three expression vectors. The system can be programmed to target virtually any DNA or RNA sequence. In addition to the second generation base editors described above, similar second generation base editors could be generated by varying the modular components of the system, including any suitable Cas orthologs, deaminase orthologs, and other DNA modification enzymes.

In some embodiments, the second target nucleic acid sequence is B2M and/or CD52. In this embodiment, the method comprises two modules, one targeting the B2M gene and the other targeting CD52.

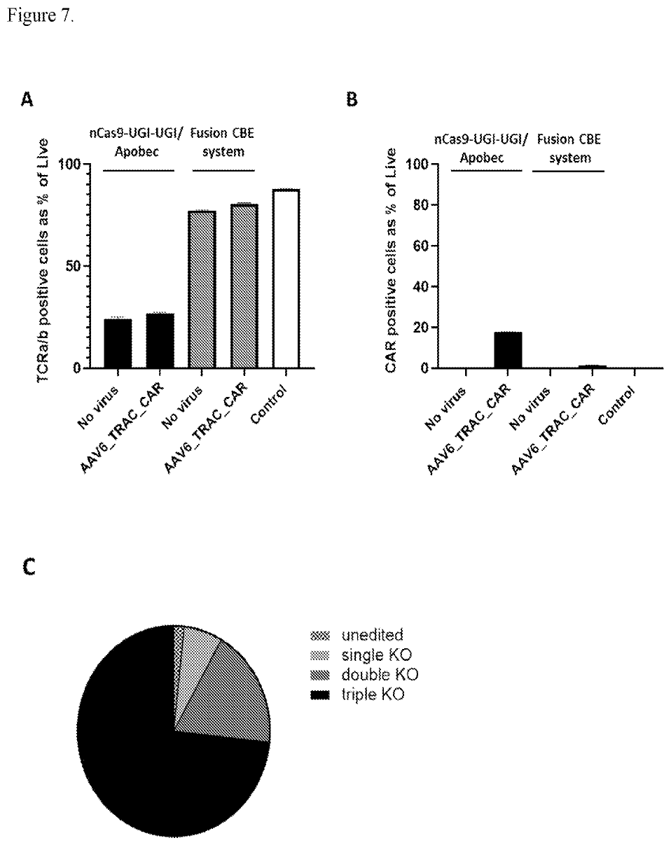

The inventors have shown that the system as described herein is significantly more effective in generating genes Knock-in compared to alternative fusion CBE systems (FIG. 7). Similarly, genes Knock-out efficiency by double nicking, according to the present disclosure is six times higher than with an alternative fusion CBE system (FIG. 7).

The present disclosure may also be used to (i) knock-out or modify genes that are involved in fratricide of immune cells, such as T cells and NK cells, or (ii) genes that alert the immune system of a subject or animal that a foreign cell, particle or molecule has entered a subject or animal, such as B2M gene or (iii) genes encoding proteins that are current therapeutic targets used to compromise or boost an immune response, such as for example, CD52 and PDCD1 genes. For example, for chimeric antigen receptor (CAR) T therapies against CD7+ leukaemias (e.g., AML), it would be necessary to genetically modify the CAR T cells so that they do not contain CD7 to avoid fratricide.

In various embodiments, the present disclosure may be used to generate knock-out of genes, modify or increase the expression of a single gene or multiple genes in various types of cells or cell lines, including but not limited to cells from mammals. The present systems and methods may be applicable to multiplex genetic modification, which, as is known in the art, involves genetically modifying multiple genes or multiple targets within the same gene. The technology may be used for many applications, including but not limited to, knock-out of genes to prevent graft versus host disease by making non-host cells non-immunogenic to the host or prevent host vs. graft disease by making non-host cells resistant to attacks by the host.

These approaches are also relevant to generating allogeneic (off-the-shelf) or autologous (patient specific) cell-based therapeutics. Such knock-out genes include, but are not limited to, the T Cell Receptor (TRAC, TRBC1, TRBC2, TRDC, TRGC1, TRGC2), the major histocompatibility complex (MHC class I and class II) genes, including B2M, co-receptors (HLA-F, HLA-G), genes involved in the innate immune response (MICA, MICB, HCP5, STING, DDX41 and Toll-like-receptors (TLRs)), inflammation (NKBBiL, LTA, TNF, LTB, LST1, NCR3, AIF1), heat shock proteins (HSPA1L, HSPA1A, HSPA1B), complement cascade, regulatory receptors (NOTCH family members), antigen processing (TAP, HLA-DM, HLA-DO), increased potency or persistence (such as PD-1, CTLA-4 and other members of the B7 family of checkpoint proteins), genes involved in immunosuppressive immune cells (such as FOXP3 and Interleukin (IL)-10), genes involved in T cell interaction with the tumour microenvironment (including but not limited to receptors of cytokines such as TGFB, IL-4, IL-7, IL-2, IL-15, IL-12, IL-18, IFNgamma), genes involved in contributing to cytokine release syndrome (including but not limited to IL-6, IFNgamma, IL-8 (CXCL8), IL-10, GM-CSF, MIP-1α/β, MCP-1 (CCL2), CXCL9, and CXCL10 (IP-10), genes that code for the antigen targeted by a CAR/TCR (for example endogenous CS1 where the CAR is designed against CS1) or other genes found to be beneficial to CAR-T/TCR-T (such as TET2, ARG2, NR4A1, NR4A2, NR4A3, TOX and TOX2) or other cell based therapeutics including but not limited to CAR-NK, CAR-B etc. See, e.g., DeRenzo et al., Genetic Modification Strategies to Enhance CAR T Cell Persistence for Patients With Solid Tumors. Front. Immunol., 15 Feb. 2019, the entire contents of which are incorporated herein by reference.

One application of the method and system provided herein is to engineer HLA alleles of bone marrow cells or bone marrow cells differentiated from iPS cells to increase haplotype match. The engineered cells can be used for bone marrow transplantation for treating leukemia. Another application is to engineer the negative regulatory element of fetal hemoglobin gene in hematopoietic stem cells for treating sickle cell anemia and beta-thalassemia. The negative regulatory element will be mutated and the expression of fetal hemoglobin gene is re-activated in hematopoietic stem cells, compensating the functional loss due to mutations in adult alpha or beta hemoglobin genes. A further application is to engineer iPS cells for generating allogeneic therapeutic cells for various degenerative diseases including Parkinson's disease (neuronal cell loss), Type 1 diabetes (pancreatic beta cell loss). Other exemplary applications include engineering HIV infection resistant T-Cells by inactivating CCR5 gene and other genes encoding receptors required for HIV entering cells; removing a premature stop codon in the DMD gene to re-establish expression of dystrophin; and the correction of cancer driver mutations, such as p53 Y163C.

Delivery of Components into Cells

In embodiments provided herein, the guide RNA molecules can be delivered to the target cell via various methods, without limitation, listed below. Firstly, direct introduction of synthetic RNA molecules (whether sgRNA, crRNA, or tracrRNA and modifications thereof) to the cell of interest by electroporation, nucleofection, transfection, via nanoparticles, via viral mediated RNA delivery, via non-viral mediated delivery, via extracellular vesicles (for example exosome and microvesicles), via eukaryotic cell transfer (for example by recombinant yeast) and other methods that can package the RNA molecules and can be delivered to the target viable cell without changes to the genomic landscape. Other methods for the introduction of guide RNA molecules include non-integrative transient transfer of DNA polynucleotides that includes the relevant sequence for the protein recruitment so that the molecule can be transcribed into the target guide RNA molecule, this includes, without limitation, DNA-only vehicles (for example, plasmids, MiniCircles, MiniVectors, MiniStrings, Protelomerase generated DNA molecules (for example Doggybones), artificial chromosome (for example HAC), cosmids), via DNA vehicles by nanoparticles, extracellular vesicles (for example exosome and microvesicles), via eukaryotic cell transfer (for example by recombinant yeast), transient viral transfer by AAV, non-integrating viral particles (for example lentivirus and retrovirus based systems), cell penetrating peptides and other technology that can mediate the introduction of DNA into a cell without direct integration into the genomic landscape. Another method for the introduction of the guide RNA include the use of integrative gene transfer technology for stable introduction of the machinery for guide RNA transcription into the genome of the target cells, this can be controlled via constitutive or promoter inducible systems to attenuate the guide RNA expression and this can also be designed so that the system can be removed after the utility has been met (for example, introducing a Cre-Lox recombination system), such technology for stable gene transfer includes, but not limited to, integrating viral particles (for example lentivirus, adenovirus, and retrovirus based systems), transposase mediate transfer (for example Sleeping Beauty and Piggybac), exploitation of the non-homologous repair pathways introduced by DNA breaks (for example utilising CRISPR and TALEN) technology and a surrogate DNA molecule, and other technology that encourages integration of the target DNA into a cell of interest.

The method for delivering the effector fusion protein and the CRISPR targeting components are often mediated by the same technology. In some situations, there are advantages to mediate the delivery of the effector fusion protein by one method and the CRISPR targeting components via another method. The applicable methods, and not limited to, are listed below. Firstly, the direct introduction of mRNA and Protein molecules directly to the cell of interest by electroporation, nucleofection, transfection, via nanoparticles, via viral mediated packaged delivery, extracellular vesicles (for example exosome and microvesicles), via eukaryotic cell transfer (for example by recombinant yeast), and other methods that can package the macromolecules and can be delivered to the target viable cell without integration into genomic landscape. Other methods for the introduction of the coding sequence of the effector fusion protein include non-integrative transient transfer of DNA polynucleotides that includes the relevant sequence for the protein recruitment so that the molecule or molecules can be transcribed and translated into the target protein molecule. This includes, without limitation, DNA-only vehicles (for example, plasmids, MiniCircles, MiniVectors, MiniStrings, Protelomerase generated DNA molecules (for example Doggybones), artificial chromosome (for example HAC), cosmids), via DNA vehicles by nanoparticles, extracellular vesicles (for example exosome and microvesicles), via eukaryotic cell transfer (for example by recombinant yeast), transient viral transfer by AAV, non-integrating viral particles (for example lentivirus and retrovirus based systems), and other technology that can mediate the introduction of DNA into a cell without direct integration into the genomic landscape. Another method for the introduction of the effector fusion protein (such as a deaminase) and/or the CRISPR targeting components includes the use of integrative gene transfer technology for stable introduction of the machinery for transcription and translation into the genome of the target cells, this can be controlled via constitutive or inducible promoter systems to attenuate the molecule, or molecules expression, and this can also be designed so that the system can be removed after the utility has been met (for example, introducing a Cre-Lox recombination system), such technology for stable gene transfer includes, but not limited to, integrating viral particles (for example lentivirus, adenovirus and retrovirus based systems), transposase mediate transfer (for example Sleeping Beauty and Piggybac), exploitation of the non-homologous repair pathways introduced by DNA breaks (for example utilising CRISPR and TALEN) technology and a surrogate DNA molecule, and other technology that encourages integration of the target DNA into a cell of interest.

### Expression System

The nucleic acids encoding the RNA scaffold, the effector fusion protein or the nickase can be cloned into one or more intermediate expression vectors for introducing into prokaryotic or eukaryotic cells for replication and/or transcription. Intermediate vectors are typically prokaryotic vectors, e.g., plasmids, or shuttle vectors, or insect vectors, for storage or manipulation of the nucleic acid encoding the RNA scaffold or protein components for production of the RNA scaffold or protein components. The nucleic acids can also be cloned into one or more expression vectors, for administration to a plant cell, animal cell. In some embodiments, the nucleic acids can be cloned into one or more expression vectors, for administration to a mammalian cell or a human cell, fungal cell, bacterial cell, or protozoan cell. Accordingly, the present disclosure provides nucleic acids that encode any of the RNA scaffold or proteins mentioned above. In some embodiments, the nucleic acids are isolated and/or purified.

The present disclosure also provides recombinant constructs or vectors having sequences encoding one or more of the RNA scaffold or proteins described above. Examples of the constructs include a vector, such as a plasmid or a viral vector, into which a nucleic acid sequence of the disclosure has been inserted, in a forward or reverse orientation. In an embodiment, the construct further includes regulatory sequences, including a promoter, operably linked to the sequence. Large numbers of suitable vectors and promoters are known to those of skill in the art, and are commercially available. Appropriate cloning and expression vectors for use with prokaryotic and eukaryotic hosts are also described in, e.g., Sambrook et al. (2001, Molecular Cloning: A Laboratory Manual, Cold Spring Harbor Press), the entire contents of which are incorporated herein by reference.

### Culturing the Cells

The method of the disclosure further comprises maintaining the cells under appropriate conditions such that the guide RNA guides the effector protein to the targeted site in the target sequence, and the effector domain modifies the target sequence. In general, the cell can be maintained under conditions appropriate for cell growth and/or maintenance. Suitable cell culture conditions are well known in the art and are described, for example, in Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001), Santiago et al. (2008) PNAS 105:5809-5814; Moehle et al. (2007) PNAS 104:3055-3060; Urnov et al. (2005) Nature 435:646-651; and Lombardo et al. (2007) Nat. Biotechnology 25:1298-1306, the entire contents of each are incorporated herein by reference. Those of skill in the art appreciate that methods for culturing cells are known in the art and can and will vary depending on the cell type. Routine optimization may be used, in all cases, to determine the best techniques for a particular cell type.

Cells useful for the methods provided herein can be freshly isolated primary cells or obtained from a frozen aliquot of a primary cell culture. In some embodiments, cells are electroporated for uptake of gRNAs and the base editing fusion protein. As described in the Examples that follow, electroporation conditions for some assays (e.g., for T cells) can comprise 1600 volts, pulse width of 10 milliseconds, 3 pulses. Following electroporation, electroporated T cells are allowed to recover in a cell culture medium and then cultured in a T cell expansion medium. In some cases, electroporated cells are allowed to recover in the cell culture medium for about 5 to about 30 minutes (e.g., about 5, 10, 15, 20, 25, 30 minutes). In one embodiment, the recovery cell culture medium is free of an antibiotic or other selection agent. In some cases, the T cell expansion medium is complete CTS OpTmizer T-cell Expansion or Immunocult-XT Expansion medium.

Various exemplary embodiments of compositions and methods according to this disclosure are now described in the following Examples:

### EXAMPLES

### Example 1. Integration of a Promoter-Less Transgene in the TRAC Locus of T Cells with Expression of the Transgene Driven by the TRAC Endogenous Promoter and Destruction of TCRα/b Expression

In this example, primary human Pan T lymphocytes were used to prove the utility of the CRISPR system targeting module (nCas9-UGI-UGI) for specific integration in the TRAC locus of a promoter-less transgene. The Pan T cells were activated utilizing anti-CD3 and anti-CD28 antibodies and then electroporated with mRNA for nCas9-UGI-UGI component and two sgRNAs targeting opposite strands in the first exon of the TRAC gene. The two single nicks generated by the nCas9-UGU-UGI at the two target loci recognised by the sgRNAs resulted in a staggered double strand break (DSB). After electroporation, cells were transduced with an AAV6 virus used to promote integration of a GFP coding sequence in frame with the TRAC gene. Integration of the transgene by homologous directed repair (HDR) or non-homologous end joining (NHEJ) induced by the DSB at this locus resulted in efficient knock-out of the TRAC gene and disruption of the TCRα/b complex. After transduction, the cells were incubated for 4-7 days and cells were checked for GFP expression and surface knock-out of TCRα/b by flow cytometry.

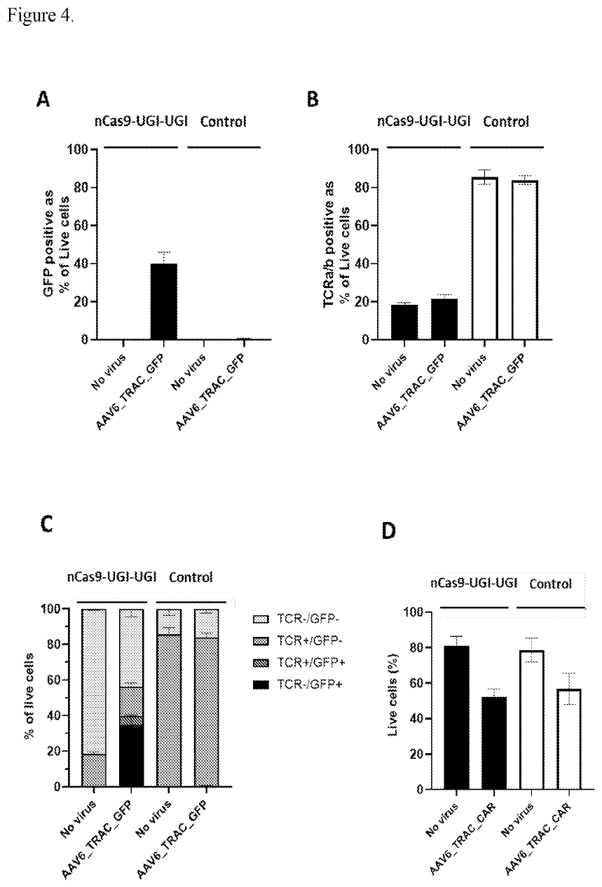

The data shows that the technology of the disclosure can efficiently induce loss of TCRα/P from the surface (FIG. 4B) and generate good level of GFP integration (FIG. 4A). Expression of GFP was not observed in the control cells. Control cells were cells that did not receive the targeting components, i.e., cells not electroporated with the nCas9-UGI-UGI component and the two sgRNAs targeting the TRAC gene. Expression of GFP was observed only in cells where the TRAC locus was cleaved and, therefore, GFP integrated and transcriptionally controlled by the TRAC promoter. As expected, the GPF expressing T cells lost the expression of TCRα/b (FIG. 4C). Furthermore, the combination of electroporation of the targeting components and AAV transduction did not affect viability when compared to transduction only, showing that the technology was not deleterious for the cells (FIG. 4D).

### Materials and Methods

### Knock-In Guide RNAs

To disrupt the TRAC locus and place the GFP under its transcriptional control, we designed sgRNA pairs targeting the 5′ end of the first exon of TRAC and an AAV vector with homology arms to the target locus and encoding a self-cleaving P2A peptide followed by the GFP cDNA. The sgRNAs to target the TRAC locus were designed following the rule of the PAM-out configuration (PAM sites faced the outside of the target region), with the cleavage sites 40-70 bp apart. The knock-in guides were designed without the 1×MS2 aptamer. The sgRNAs were synthesized by Horizon Discovery (formerly Dharmacon).

Synthetic sgRNA sequences
(SEQ ID NO: 29)
mN*mN*NNNNNNNNNNNNNNNNNNGUUUUAGAGCUAGAAAUAGCAAGUU
AAAAUAAGGCUAGUCCGUUAUCAACUUGAAAAAGUGGCACCGAGUCGGU
GCUmU*mU*U
(m) 2-O Methyl and (*) phosphorothioate modified
residues N (spacer) = any of G, U, A or C
5′ sgRNA sequence
(SEQ ID NO: 30)
mG*mA*GAAUCAAAAUCGGUGAAUGUUUUAGAGCUAGAAAUAGCAAGUU
AAAAUAAGGCUAGUCCGUUAUCAACUUGAAAAAGUGGCACCGAGUCGGU
GCUmU*mU*U
3′ sgRNA sequence
(SEQ ID NO: 31)
mA*mA*CAAAUGUGUCACAAAGUAGUUUUAGAGCUAGAAAUAGCAAGUU
AAAAUAAGGCUAGUCCGUUAUCAACUUGAAAAAGUGGCACCGAGUCGGU
GCUmU*mU*U

### Messenger RNA

Messenger RNA molecules were custom synthesized by TriLink Biotechnologies utilizing the modified nucleotides pseudouridine and 5-Methyl-Cytosine. The mRNA component translated to the following proteins: nCas9=NLS-nCas9-UGI-UGI-NLS.

### AAV Plasmid Construction

The AAV plasmids were custom synthesized by GenScript. Based on a pAAV backbone, we designed the pAAV-TRAC-GFP containing in order: 0.9 kb left homology arm of genomic TRAC flanking the 5′ gRNA targeting sequence, a GSG (gly-ser-gly) peptide followed by a self-cleaving P2A peptide in frame with the first exon of TRAC, a GFP coding sequence, the bovine growth hormone polyA signal (bGHpA) and 0.9kb right homology arm of the genomic TRAC flanking the 3′ gRNA targeting sequence.

### Cells

CD3+ T cells were either isolated from whole blood or outsourced from Hemacare. Briefly, peripheral blood mononuclear cells were isolated by density gradient centrifugation (SepMate PBMC isolation tube, STEMCELL Technologies) and T lymphocytes were then purified using the EasySep™ Human T Cell Isolation Kit (STEMCELL Technologies). Cells were activated with Dynabeads (1:1 beads:cell) Human T-Activator CD3/CD28 (ThermoFisher) in Immunocult XT T Cell Expansion medium (STEMCELL Technologies) supplemented with 100 U/ml IL-2 (STEMCELL Technologies) and 1× Penicillin/Streptomycin (Thermofisher) at 37° C. and 5% CO₂ for 48 hours at a density of 10⁶ cells per ml. Post-activation, beads were removed by placement on a magnet and the cells were transferred back into culture.

### T Cell Electroporation

After 48-72 hours post-activation T cells were electroporated using the Neon Electroporator (Thermofisher). Neon Electroporator conditions were 1600v/10 ms/3 pulses with a 10 μl tip with 250 k cells, combined total of mRNA amount of 1-5 μg and 2 μM each of the targeting gRNAs. Post-electroporation cells were transferred to Immunocult XT media with 100 U/ml IL-2, 100 U/ml IL-7 and 100 U/ml IL-15 (STEMCELL Technologies) and cultured at 37° C. and 5% CO₂ for 48-72 hours.

### T Cell Transduction

Recombinant AAV6 particles were generated by Vigene Biosciences. Where applicable, recombinant AAV6 particles carrying the GFP coding sequence were added to the culture 2 to 4 h after electroporation, at the 1×10⁶ genome copies (GC) per cell. Subsequently, edited cells were cultured at 37° C. and 5% CO₂ for 96 hours, maintaining the density of ˜1×10⁶ cells per ml.

### Flow Cytometry

T cell identity and QC were confirmed by CD3 antibody staining (Biolegend). T cell activation was confirmed by CD25 staining. GFP positive cells were measured by flow cytometry at 7 days post electroporation/transduction. Levels of TCR−/GFP+ cells were assessed at 7 days post electroporation/transduction by flow cytometry using a TCRα/β antibody (Biolegend).

Any phenotype data was reported as percentage of viable cells, as ascertained by viability dye staining.

### Example 2. Integration of a CAR Gene in the TRAC Locus of T Cells with Expression of the Transgene Driven by the TRAC Endogenous Promoter and Destruction of TCR Expression

In this example, primary human Pan T lymphocytes were used to prove the utility of the enzyme in the CRISPR system, the targeting module, (nCas9-UGI-UGI) for specific integration in the TRAC locus of a CAR gene. The Pan T cells were activated utilizing anti-CD3 and anti-CD28 antibodies and then electroporated with mRNA for nCas9-UGI-UGI component and two sgRNAs targeting opposite strands in the first exon of the TRAC gene. The two single nicks generated by the nCas9-UGU-UGI at the two target loci recognised by the sgRNAs resulted in a staggered double strand break (DSB). After electroporation, cells were transduced with an AAV6 virus used to promote integration of a CAR coding sequence in frame with the TRAC gene. Integration of the transgene by homologous directed repair (HDR) or non-homologous end joining (NHEJ) induced by the DSB at this locus resulted in efficient knock-out of the TRAC gene and disruption of the TCR complex. After transduction, the cells were incubated for 4-7 days and cells were checked for CAR expression and surface knock-out of TCR by flow cytometry.

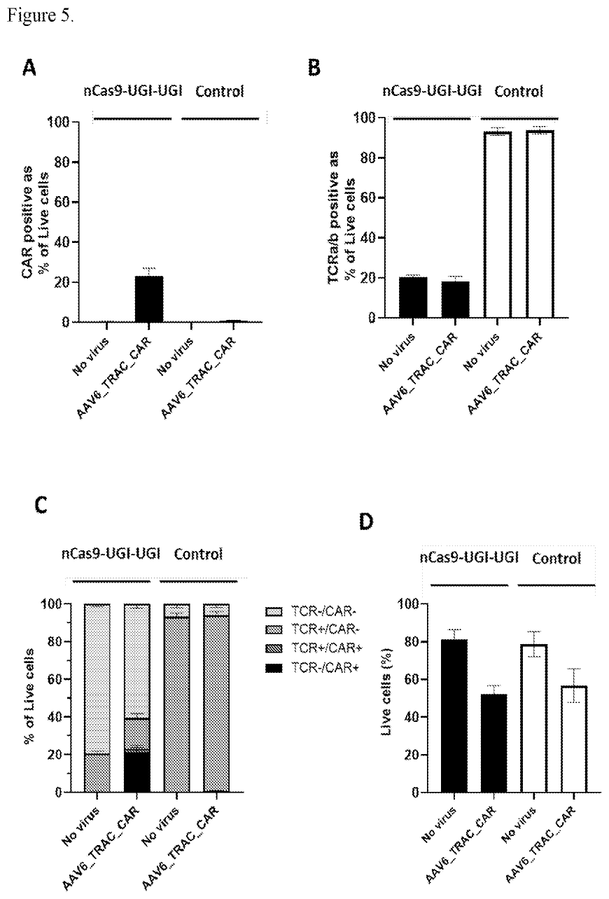

The data shows that the technology of the disclosure can efficiently induce loss of TCRα/β from the surface and generate a good level of CAR integration (FIG. 5 A-B). Expression of CAR was not observed in control cells (cells that did not receive the nCas9-UGI-UGI and two sgRNAs targeting the TRAC gene components), but only in cells where the TRAC locus was cleaved and therefore CAR integrated and transcriptionally controlled by the TRAC promoter. As expected, the CAR expressing T cells lost the expression of TCR (FIG. 5C). Furthermore, the combination of electroporation of the targeting components and AAV transduction did not affect viability when compared to transduction only, showing that the technology was not deleterious for the cells (FIG. 5D).

### Materials and Methods

### Knock-In Guide RNAs

To disrupt the TRAC locus and place the CD19-specific 1928z CAR gene under its transcriptional control, we designed sgRNA pairs targeting the 5′ end of the first exon of TRAC and an AAV vector with homology arms to the target locus and encoding a self-cleaving P2A peptide followed by the CAR cDNA. The sgRNAs to target the TRAC locus were designed following the rule of the PAM-out configuration (PAM sites faced the outside of the target region), with the cleavage sites 40-70 bp apart. The knock-in guides were designed without the 1×MS2 aptamer. The sgRNAs were synthesised by Horizon Discovery (formerly Dharmacon).

Synthetic sgRNA sequences
(SEQ ID NO: 29)
mN*mN*NNNNNNNNNNNNNNNNNNGUUUUAGAGCUAGAAAUAGCAAGUU
AAAAUAAGGCUAGUCCGUUAUCAACUUGAAAAAGUGGCACCGAGUCGGU
GCUmU*mU*U
(m) 2-O Methyl and (*) phosphorothioate modified
residues
5′ sgRNA sequence
(SEQ ID NO: 30)
mG*mA*GAAUCAAAAUCGGUGAAUGUUUUAGAGCUAGAAAUAGCAAGUU
AAAAUAAGGCUAGUCCGUUAUCAACUUGAAAAAGUGGCACCGAGUCGGU
GCUmU*mU*U
3′ sgRNA sequence
(SEQ ID NO: 31)
mA*mA*CAAAUGUGUCACAAAGUAGUUUUAGAGCUAGAAAUAGCAAGUU
AAAAUAAGGCUAGUCCGUUAUCAACUUGAAAAAGUGGCACCGAGUCGGU
GCUmU*mU*U

### Messenger RNA

Messenger RNA molecules were custom synthesized by TriLink Biotechnologies utilizing the modified nucleotides pseudouridine and 5-Methyl-Cytosine. The mRNA component translated to the following proteins: nCas9=NLS-nCas9-UGI-UGI-NLS.

### AAV Plasmid Construction

The AAV plasmids were custom synthesized by GenScript. Based on a pAAV backbone we designed the pAAV-TRAC-1928Z_CAR and the pAAV-TRAC-GFP containing in order: 0.9 kb left homology arm of genomic TRAC flanking the 5′ gRNA targeting sequence, a GSG (gly-ser-gly) peptide followed by a self-cleaving P2A peptide in frame with the first exon of TRAC, the 1928z CAR used in Yescarta™ therapy or a GFP coding sequence, the bovine growth hormone polyA signal (bGHpA) and 0.9kb right homology arm of the genomic TRAC flanking the 3′ gRNA targeting sequence. Briefly, the CD19-CAR (Kochenderfer et al 2009, J Immunotherapy) comprised a single chain variable fragment scFV specific for the human CD19 derived from the FMC63 mouse hybridoma (Nicholson et al 1997, Mol Immunology), a portion of the human CD28 molecule (a hinge extracellular part, a transmembrane domain and the entire intracellular domain) and the entire domain of CD3-zeta chain (FIG. 2).

### Cells

CD3+ T cells were either isolated from whole blood or outsourced from Hemacare. Briefly, peripheral blood mononuclear cells were isolated by density gradient centrifugation (SepMate PBMC isolation tube, STEMCELL Technologies) and T lymphocytes were then purified using the EasySep™ Human T Cell Isolation Kit (STEMCELL Technologies). Cells were activated with Dynabeads (1:1 beads:cell) Human T-Activator CD3/CD28 (ThermoFisher) in Immunocult XT T Cell Expansion medium (STEMCELL Technologies) supplemented with 100 U/ml IL-2 (STEMCELL Technologies) and 1x Penicillin/Streptomycin (Thermofisher) at 37° C. and 5% CO₂ for 48 hours at a density of 10⁶ cells per ml. Post-activation, beads were removed by placement on a magnet and the cells were transferred back into culture.

### T Cell Electroporation

After 48-72 hours post-activation, T cells were electroporated using the Neon Electroporator (Thermofisher). Neon Electroporator conditions were 1600v/10 ms/3 pulses with a 10 μl tip with 250 k cells, combined total of mRNA amount of 1-5 μg and 2 μM each of the targeting gRNAs. Post-electroporation, cells were transferred to Immunocult XT media with 100 U/ml IL-2, 100 U/ml IL-7 and 100 U/ml IL-15 (STEMCELL Technologies) and cultured at 37° C. and 5% CO₂ for 48-72 hours.

### T Cell Transduction

Recombinant AAV6 particles were generated by Vigene Biosciences. Where applicable, recombinant AAV6 particles carrying the CD19-CAR coding sequence were added to the culture 2 to 4 h after electroporation, at the 1×10⁶ GC per cell. Subsequently, edited cells were cultured at 37° C. and 5% CO₂ for 96 hours, maintaining the density of ˜1×10⁶ cells per ml.

### Flow Cytometry

T cell identity and QC was confirmed by CD3 antibody staining (Biolegend). T cell activation was confirmed by CD25 staining. CD19-CAR positive cells were detected by flow cytometry using an Anti-FMC63 scFv Antibody (AcroBiosystem) at 96 hours post electroporation/transduction. Levels of TCR−/CAR+ cells were assessed by combined staining with a TCRa/b antibody (Biolegend). Any phenotype data was reported as percentage of viable cells, as ascertained by viability dye staining.

### Example 3. Generation of Universal CAR-T Cells by the Aptamer-Based Base Editing System

In this example, primary human Pan T lymphocytes were used to prove the utility of the CRISPR-aptamer based gene editing system for specific integration in the TRAC locus of a CAR gene and simultaneous knock-out of TRAC, B2M and CD52 genes by the cytosine base editing system. With the method of the present disclosure, base editing guided knock-out was achieved through recruitment of the deaminase on the target site by a sgRNA-aptamer. CAR integration and consequent TRAC knock-out was achieved through the same enzyme used for the CRISPR system, combined with sgRNAs. The advantage over previous systems is that one CRISPR enzyme, or single RNA guided nickase, was used to achieve both the modifications, i.e., the same enzyme was used for the CAR gene knock-in and for the TRAC, B2M and CD52 genes knock-out.

The Pan T cells were activated utilizing anti-CD3 and anti-CD28 antibodies and then electroporated with the following components: (i) mRNA encoding the deaminase-MCP, (ii) mRNA encoding nCas9-UGI-UGI protein, (iii) two sgRNAs targeting opposite strands in the first exon of the TRAC gene and (iv) sgRNA-aptamer for two different genes. The two single nicks generated by the nCas9-UGU-UGI at the two target loci recognized by the sgRNAs resulted in staggered double strand breaks (DSB). After electroporation, cells were transduced with an AAV6 virus used to promote integration of a CAR coding sequence in frame with the TRAC gene by homologous directed repair (HDR). Integration of the transgene by HDR or non-homologous end joining (NHEJ) induced by the DSB at this locus resulted in efficient knock-out of the TRAC gene and disruption of the TCR complex. After transduction, the cells were incubated for 4-7 days and were then checked for CAR expression and surface knock-out of TCR, B2M and CD52 by flow cytometry. Base conversion was measured by targeted PCR amplification and Sanger sequencing. Multi-antibody panel was used to ascertain multiplex KO level within the CAR+population by flow cytometry.

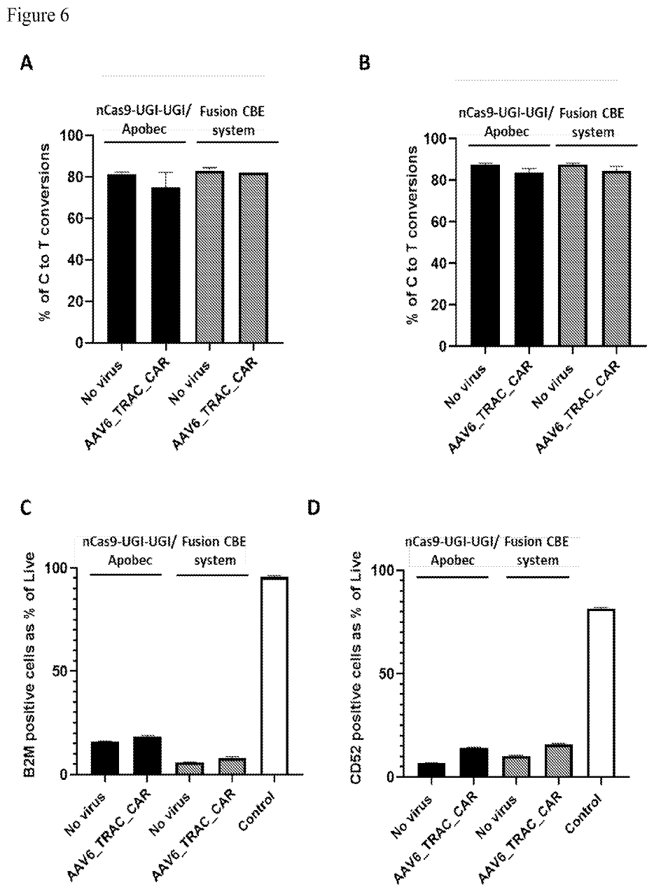

When base editing components were delivered to the cells, high levels of base conversion were observed at the two targeted loci, B2M and CD52, and editing efficiency was not compromised by the viral vector delivery (FIG. 6A-B). The data shows that sgRNA-aptamer-based base editing was comparable in efficiency to a traditional CRISPR-assisted base-editing system, where the deaminase is fused to the Cas protein. Functional KO information generated by flow cytometry correlated with the base conversion (FIG. 6C-D). However, loss of TCRα/β from the surface and CAR integration was efficiently achieved only by the enzyme of the CRISPR system of the present disclosure, not by the alternative fusion CBE system (FIG. 7A-B). High level of multiple gene KO (triple KO in this example) was achieved by the present technology in the CAR expressing population (FIG. 7C). This data shows that the present technology generated CAR-T cells with functional knock-out in multiple genes that work as universal CAR-T cells. The data also shows the superiority of the CRISPR based gene editing system of the present disclosure to achieve HDR guided integration compared to the alternative fusion CBE system.

### Materials and Methods

### Base Editing Guide RNAs

Internally generated data was used to specify base editing windows calculated at set distances from the PAM motif (NGG). The data was used to develop algorithms to predict Phenotype or Gene KO applicable guides sequence for the following genes: TRAC, TRBC1, TRBC2, PDCD-1, B2M, and CD52 (Table 4). The sgRNAs were designed including the 1×MS2 aptamer. The guide RNA sequences were synthesised by Horizon Discovery (formerly Dharmacon) and Agilent.

Synthetic 1xMS2 sgRNA sequence
(SEQ ID NO: 32)
mN*mN*NNNNNNNNNNNNNNNNNNGUUUUAGAGCUAGAAAUAGCAAGUU
AAAAUAAGGCUAGUCCGUUAUCAACUUGAAAAAGUGGCACCGAGUCGGU
GCGCGCACAUGAGGAUCACCCAUGUGCUUUUmU*mU*U
(m) 2-O Methyl and (*) phosphorothioate modified
residues

TABLE 4
crRNA sequences in the single-guide RNAs (sgRNAs) for TRAC, TRBC1, TRBC2,
PDCD1, CD52 and B2M genes. An example list of guide designs for guide RNA sequences that
can create a functional knock-out using the base editing technology exemplified. The list
includes guides specific to the introduction of a premature stop codon and splice
disruption sites, which were generated by an in-house proprietary software.

| Gene | | KO | | | SEQ ID | |
|---|---|---|---|---|---|---|
| Name | Guide ID | Type* | Strand | Guide Sequence | NO: | PAM |
| B2M | B2M_1 | Stop | sense | CACAGCCCAAGATAGTTAAG | 33 | TGG |
| | B2M_2 | Stop | sense | ACAGCCCAAGATAGTTAAGT | 34 | GGG |
| | B2M_3 | Stop | anti | TTACCCCACTTAACTATCTT | 35 | GGG |
| | B2M_4 | Stop | anti | CTTACCCCACTTAACTATCT | 36 | TGG |
| | B2M_5 | Splice | anti | ACTCACGCTGGATAGCCTCC | 37 | AGG |
| | B2M_6 | Splice | anti | TTGGAGTACCTGAGGAATAT | 38 | CGG |
| | B2M_7 | Splice | anti | TCGATCTATGAAAAAGACAG | 39 | TGG |
| | B2M_8 | Splice | anti | AACCTGAAAAGAAAAGAAAA | 40 | AGG |
| CD52 | CD52_1 | Stop | sense | GTACAGGTAAGAGCAACGCC | 41 | TGG |
| | CD52_2 | Stop | sense | CTCCTCCTACAGATACAAAC | 42 | TGG |
| | CD52_3 | Stop | sense | CAGATACAAACTGGACTCTC | 43 | AGG |
| | CD52_4 | Splice | anti | CTCTTACCTGTACCATAACC | 44 | AGG |
| | CD52_5 | Splice | anti | GTATCTGTAGGAGGAGAAGT | 45 | GGG |
| | CD52_6 | Splice | anti | TGTATCTGTAGGAGGAGAAG | 46 | TGG |
| | CD52_7 | Splice | anti | GTCCAGTTTGTATCTGTAGG | 47 | AGG |
| TRAC | TRAC_1 | Stop | sense | AACAAATGTGTCACAAAGTA | 48 | AGG |
| | TRAC_2 | Stop | sense | CTTCTTCCCCAGCCCAGGTA | 49 | AGG |
| | TRAC_3 | Stop | sense | TTCTTCCCCAGCCCAGGTAA | 50 | GGG |
| | TRAC_4 | Stop | sense | AGCCCAGGTAAGGGCAGCTT | 51 | TGG |
| | TRAC_5 | Stop | sense | TTTCAAAACCTGTCAGTGAT | 52 | TGG |
| | TRAC_6 | Stop | sense | TTCAAAACCTGTCAGTGATT | 53 | GGG |
| | TRAC_7 | Stop | sense | CCGAATCCTCCTCCTGAAAG | 54 | TGG |
| | TRAC_8 | Splice | anti | CTTACCTGGGCTGGGGAAGA | 55 | AGG |
| | TRAC_9 | Splice | anti | TTCGTATCTGTAAAACCAAG | 56 | AGG |
| TRBC1/2 | TRBC1/2_1 | Stop | sense | CCACACCCAAAAGGCCACAC | 57 | TGG |
| | TRBC1/2_2 | Stop | anti | CCCACCAGCTCAGCTCCACG | 58 | TGG |
| | TRBC1/2_3 | Stop | sense | CGCTGTCAAGTCCAGTTCTA | 59 | CGG |
| | TRBC1/2_4 | Stop | sense | GCTGTCAAGTCCAGTTCTAC | 60 | GGG |
| | TRBC1/2_5 | Stop | sense | AGTCCAGTTCTACGGGCTCT | 61 | CGG |
| | TRBC1/2_6 | Stop | sense | CACCCAGATCGTCAGCGCCG | 62 | AGG |
| | TRBC1/2_7 | Splice | anti | ACCTGCTCTACCCCAGGCCT | 63 | CGG |
| | TRBC1/2_8 | Splice | anti | CCACTCACCTGCTCTACCCC | 64 | AGG |
| TRBC1 | TRBC1_1 | Stop | sense | CACGGACCCGCAGCCCCTCA | 65 | AGG |
| | TRBC1_2 | Stop | anti | GCGGGGGTTCTGCCAGAAGG | 66 | TGG |
| | TRBC1_3 | Stop | anti | GTTGCGGGGGTTCTGCCAGA | 67 | AGG |
| | TRBC1_4 | Stop | sense | ATGACGAGTGGACCCAGGAT | 68 | AGG |
| | TRBC1_5 | Stop | sense | TGACGAGTGGACCCAGGATA | 69 | GGG |
| | TRBC1_6 | Stop | anti | ACCTGCTCTACCCCAGGCCT | 70 | CGG |
| | TRBC1_7 | Stop | sense | CCAACAGTGTCCTACCAGCA | 71 | AGG |
| | TRBC1_8 | Stop | sense | CAACAGTGTCCTACCAGCAA | 72 | GGG |
| | TRBC1_9 | Stop | sense | AACAGTGTCCTACCAGCAAG | 73 | GGG |
| | TRBC1_10 | Splice | anti | GTCTGAAAGAAAGCAGGGAG | 74 | AGG |
| | TRBC1_11 | Splice | anti | CCACAGTCTGAAAGAAAGCA | 75 | GGG |
| | TRBC1_12 | Splice | anti | GCCACAGTCTGAAAGAAAGC | 76 | AGG |
| | TRBC1_13 | Splice | anti | GACACTGTTGGCACGGAGGA | 77 | AGG |
| | TRBC1_14 | Splice | anti | GTAGGACACTGTTGGCACGG | 78 | AGG |
| | TRBC1_15 | Splice | anti | TACCATGGCCATCAACACAA | 79 | GGG |
| | TRBC1_16 | Splice | anti | TTACCATGGCCATCAACACA | 80 | AGG |
| TRBC2 | TRBC2_1 | Stop | anti | CCAGCTCAGCTCCACGTGGT | 81 | CGG |
| | TRBC2_2 | Stop | sense | CACAGACCCGCAGCCCCTCA | 82 | AGG |
| | TRBC2_3 | Stop | anti | GCGGGGGTTCTGCCAGAAGG | 83 | TGG |
| | TRBC2_4 | Stop | anti | GTTGCGGGGGTTCTGCCAGA | 84 | AGG |
| | TRBC2_5 | Stop | sense | ATGACGAGTGGACCCAGGAT | 85 | AGG |
| | TRBC2_6 | Stop | sense | TGACGAGTGGACCCAGGATA | 86 | GGG |
| | TRBC2_7 | Stop | anti | ACCTGCTCTACCCCAGGCCT | 87 | CGG |
| | TRBC2_8 | Stop | sense | TCAACAGAGTCTTACCAGCA | 88 | AGG |
| | TRBC2_9 | Stop | sense | CAACAGAGTCTTACCAGCAA | 89 | GGG |
| | TRBC2_10 | Stop | sense | AACAGAGTCTTACCAGCAAG | 90 | GGG |
| | TRBC2_11 | Splice | anti | CACAGTCTGAAAGAAAACAG | 91 | AGG |
| | TRBC2_12 | Splice | anti | CCACAGTCTGAAAGAAAACA | 92 | AGG |
| | TRBC2_13 | Splice | anti | GCCACAGTCTGAAAGAAAAC | 93 | AGG |
| PDCD1 | PDCD1_1 | Stop | sense | TCCAGGCATGCAGATCCCAC | 94 | AGG |
| | PDCD1_2 | Stop | sense | TGCAGATCCCACAGGCGCCC | 95 | TGG |
| | PDCD1_3 | Stop | anti | CGACTGGCCAGGGCGCCTGT | 96 | GGG |
| | PDCD1_4 | Stop | anti | ACGACTGGCCAGGGCGCCTG | 97 | TGG |
| | PDCD1_5 | Stop | anti | ACCGCCCAGACGACTGGCCA | 98 | GGG |
| | PDCD1_6 | Stop | anti | CACCGCCCAGACGACTGGCC | 99 | AGG |
| | PDCD1_7 | Stop | anti | TGTAGCACCGCCCAGACGAC | 100 | TGG |
| | PDCD1_8 | Stop | sense | GGGCGGTGCTACAACTGGGC | 101 | TGG |
| | PDCD1_9 | Stop | sense | CGGTGCTACAACTGGGCTGG | 102 | CGG |
| | PDCD1_10 | Stop | sense | CTACAACTGGGCTGGCGGCC | 103 | AGG |
| | PDCD1_11 | Stop | anti | CACCTACCTAAGAACCATCC | 104 | TGG |
| | PDCD1_12 | Stop | anti | GGGGTTCCAGGGCCTGTCTG | 105 | GGG |
| | PDCD1_13 | Stop | anti | GGGGGTTCCAGGGCCTGTCT | 106 | GGG |
| | PDCD1_14 | Stop | anti | GGGGGGTTCCAGGGCCTGTC | 107 | TGG |
| | PDCD1_15 | Stop | sense | CAGCAACCAGACGGACAAGC | 108 | TGG |
| | PDCD1_16 | Stop | sense | CCCGAGGACCGCAGCCAGCC | 109 | CGG |
| | PDCD1_17 | Stop | sense | GGACCGCAGCCAGCCCGGCC | 110 | AGG |
| | PDCD1_18 | Stop | sense | CGTGTCACACAACTGCCCAA | 111 | CGG |
| | PDCD1_19 | Stop | sense | GTGTCACACAACTGCCCAAC | 112 | GGG |
| | PDCD1_20 | Stop | sense | CGCAGATCAAAGAGAGCCTG | 113 | CGG |
| | PDCD1_21 | Stop | sense | GCAGATCAAAGAGAGCCTGC | 114 | GGG |
| | PDCD1_22 | Stop | sense | AGCCGGCCAGTTCCAAACCC | 115 | TGG |
| | PDCD1_23 | Stop | sense | CGGCCAGTTCCAAACCCTGG | 116 | TGG |
| | PDCD1_24 | Stop | sense | CAGTTCCAAACCCTGGTGGT | 117 | TGG |
| | PDCD1_25 | Stop | anti | GGACCCAGACTAGCAGCACC | 118 | AGG |
| | PDCD1_26 | Splice | anti | CACCTACCTAAGAACCATCC | 119 | TGG |
| | PDCD1_27 | Splice | anti | GGAGTCTGAGAGATGGAGAG | 120 | AGG |
| | PDCD1_28 | Splice | anti | TCTGGAAGGGCACAAAGGTC | 121 | AGG |
| | PDCD1_29 | Splice | anti | TTCTCTCTGGAAGGGCACAA | 122 | AGG |
| | PDCD1_30 | Splice | anti | TGACGTTACCTCGTGCGGCC | 123 | CGG |
| | PDCD1_31 | Splice | anti | TCCCTGCAGAGAAACACACT | 124 | TGG |
| | PDCD1_32 | Splice | anti | GAGACTCACCAGGGGCTGGC | 125 | CGG |
| | PDCD1_33 | Splice | anti | TCTTTGAGGAGAAAGGGAGA | 126 | GGG |
| | PDCD1_34 | Splice | anti | TTCTTTGAGGAGAAAGGGAG | 127 | AGG |

*Stop = Premature stop codon, Splice = Splice site disruption

### Knock-In Guide RNAs

To disrupt the TRAC locus and place the CD19-specific 1928z CAR gene under its transcriptional control, we designed sgRNA pairs targeting the 5′ end of the first exon of TRAC and an AAV vector with homology arms to the target locus and encoding a self-cleaving P2A peptide followed by the CAR cDNA. The sgRNAs to target the TRAC locus were designed following the rule of the PAM-out configuration (PAM sites faced the outside of the target region), with the cleavage sites 40-70 bp apart. The knock-in guides were designed without the 1×MS2 aptamer. The sgRNAs were synthesized by Horizon Discovery (formerly Dharmacon).

Synthetic sgRNA sequences
(SEQ ID NO: 29)
mN*mN*NNNNNNNNNNNNNNNNNNGUUUUAGAGCUAGAAAUAGCAAGUU
AAAAUAAGGCUAGUCCGUUAUCAACUUGAAAAAGUGGCACCGAGUCGGU
GCUmU*mU*U
(m) 2-O Methyl and (*) phosphorothioate modified
residues
5′ sgRNA sequence
(SEQ ID NO: 30)
mG*mA*GAAUCAAAAUCGGUGAAUGUUUUAGAGCUAGAAAUAGCAAGUU
AAAAUAAGGCUAGUCCGUUAUCAACUUGAAAAAGUGGCACCGAGUCGGU
GCUmU*mU*U
3′ sgRNA sequence
(SEQ ID NO: 31)
mA*mA*CAAAUGUGUCACAAAGUAGUUUUAGAGCUAGAAAUAGCAAGUU
AAAAUAAGGCUAGUCCGUUAUCAACUUGAAAAAGUGGCACCGAGUCGGU
GCUmU*mU*U

### Messenger RNA

Messenger RNA molecules were custom synthesized by TriLink Biotechnologies utilising the modified nucleotides pseudouridine and 5-Methyl-Cytosine. The mRNA components translated to the following proteins: Deaminase Apobec 1=NLS-rApobec1-Linker-MCP and nCas9=NLS-nCas9-UGI-UGI-NLS.

### AAV Plasmid Construction

The AAV plasmids were custom synthesized by GenScript. Based on a pAAV backbone we designed the pAAV-TRAC-1928Z_CAR containing in order: 0.9 kb left homology arm of genomic TRAC flanking the 5′ gRNA targeting sequence, a GSG (gly-ser-gly) peptide followed by a self-cleaving P2A peptide in frame with the first exon of TRAC, the 1928z CAR used in Yescarta™ therapy, the bovine growth hormone polyA signal (bGHpA) and 0.9kb right homology arm of the genomic TRAC flanking the 3′ gRNA targeting sequence. Briefly, the CD19-CAR (Kochenderfer et al. 2009, J Immunotherapy) comprised a single chain variable fragment scFV specific for the human CD19 derived from the FMC63 mouse hybridoma (Nicholson et al. 1997, Mol Immunology), a portion of the human CD28 molecule (a hinge extracellular part, a transmembrane domain and the entire intracellular domain) and the entire domain of CD3-zeta chain. A second AAV vector was designed and cloned where the CD19-CAR CDS was replaced by the turboGFP coding sequence.

### Cells

CD3+ T cells were either isolated from whole blood or outsourced from Hemacare. Briefly, peripheral blood mononuclear cells were isolated by density gradient centrifugation (SepMate PBMC isolation tube, STEMCELL Technologies) and T lymphocytes were then purified using the EasySep™ Human T Cell Isolation Kit (STEMCELL Technologies). Cells were activated with Dynabeads (1:1 beads:cell) Human T-Activator CD3/CD28 (ThermoFisher) in Immunocult XT T Cell Expansion medium (STEMCELL Technologies) supplemented with 100 U/ml IL-2 (STEMCELLTechnologies) and 1× Penicillin/Streptomycin (Thermofisher) at 37° C. and 5% CO₂ for 48 hours at a density of 10⁶ cells per ml. Post-activation, beads were removed by placement on a magnet and the cells were transferred back into culture.

### T Cell Electroporation

After 48-72 hours post-activation, T cells were electroporated using the Neon Electroporator (Thermofisher). Neon Electroporator conditions were 1600v/10 ms/3 pulses with a 10 μl tip with 250 k cells, combined total of mRNA amount of 1-5 μg, for both the Deaminase-MCP and nCas9-UGI-UGI, and where applicable 2 μM each of the targeting gRNAs. Post-electroporation cells were transferred to Immunocult XT media with 100 U/ml IL-2, 100 U/ml IL-7 and 100 U/ml IL-15 (STEMCELLTechnologies) and cultured at 37° C. and 5% CO₂ for 48-72 hours.

### T Cell Transduction

Recombinant AAV6 particles were generated by Vigene Biosciences. Where applicable, recombinant AAV6 particles were added to the culture 2 to 4 h after electroporation, at the 1×10⁶ GC per cell. Subsequently, edited cells were cultured at 37 C and 5% CO2 for 96 hours, maintaining the density of ˜1×10⁶ cells per ml.

### Flow Cytometry

T cell identity and QC was confirmed by CD3 antibody staining (Biolegend). T cell activation was confirmed by CD25 staining. CD19-CAR+ cells were detected by flow cytometry using an Anti-FMC63 scFv Antibody (AcroBiosystem) at 96 hours post electroporation/transduction. Phenotypic Gene Multiplex KO was assessed at 96 hours post electroporation/transduction: TRAC was confirmed by TCRab antibody staining (Biolegend), B2M by B2M-Antibody (Biolegend) and CD52 with a CD52-antibody (Biolegend); any phenotype data was reported as percentage of viable cells, as ascertained by viability dye staining.

### Genomic DNA Analysis

Genomic DNA was released from lysed cells 96 hours post-electroporation. Loci of interest were amplified by PCR and products then sent for Sanger sequencing (Genewiz). Data was analyzed by a proprietary in-house software.

TABLE 5
Single guide RNA for knock-in integration in the TRAC locus. An example list of
guide designs for guide RNA sequences that, when used as a pair and combined with
the nCas9-UGI-UGI of the Examples, generate two nicks in opposite strands, creating
a functional knock-out and inducing site-specific integration using the base editing
technology exemplified and a template transgene with homology arms to the locus.
Single guide RNA with PAM located 5′ to the integration site have to be combined with
single guide RNA with PAM located 3′ to the integration site.

| | | | PAM location | | | |
|---|---|---|---|---|---|---|
| | | | to the | | | |
| Gene | | | integration | | SEQ | |
| Name | Guide ID | Strand | site | Spacer Sequence | ID: | PAM |
| TRAC | TRAC_5′_1 | anti | 5′ | TCAGGGTTCTGGATATCTGT | 128 | GGG |
| | TRAC_5′_2 | anti | 5′ | CTCTCAGCTGGTACACGGCA | 129 | GGG |
| | TRAC_5′_3 | anti | 5′ | AGCTGGTACACGGCAGGGTC | 130 | AGG |
| | TRAC_5′_4 | anti | 5′ | ACACGGCAGGGTCAGGGTTC | 131 | TGG |
| | TRAC_5′_5 | anti | 5′ | GAGAATCAAAATCGGTGAAT | 132 | AGG |
| | TRAC_3′_1 | sense | 3′ | AACAAATGTGTCACAAAGTA | 133 | AGG |
| | TRAC_3′_2 | sense | 3′ | TGTGCTAGACATGAGGTCTA | 134 | TGG |

### Example 4. Generation of iPSC Cell Lines for Allogeneic CAR Therapy by CRISPR-Aptamer Based Gene Editing System

In this example, induced pluripotent stem cells (iPSCs) are used to prove the utility of the CRISPR-aptamer based gene editing system for specific integration in the TRAC locus or B2M locus of a CAR gene and simultaneous knock-out of TRAC, B2M and CIITA genes. With the method of the present disclosure, base editing guided knock-out is achieved through recruitment of the deaminase on the target site by a sgRNA-aptamer. CAR integration and consequent B2M knock-out is achieved through the same enzyme used for the CRISPR system (combined with sgRNAs) and the base editing system. The advantage over previous systems is that one enzyme or single RNA guided nickase is used to achieve all the modifications, i.e., the same enzyme is used for the CAR gene knock-in and for the TRAC and CIITA genes knock-out.

The iPSCs are cultivated in cell line specific media, disassociated, and then electroporated with mRNA components for both the deaminase-MCP, nCas9-UGI-UGI components, two sgRNAs targeting opposite strands in the first exon of the B2M gene, sgRNA-aptamer for two different genes (TRAC and CIITA), and an exogenous dsDNA template. The two single nicks generated by the nCas9-UGU-UGI at the two target loci recognized by the sgRNAs result in a staggered double strand break (DSB). The exogenous dsDNA template contained homology arms relevant to the DNA break target site on the B2M locus and also contained the CAR transgene cassette. After electroporation, the cells are incubated for 4-7 days and are then checked for CAR expression and knock-out of B2M and CIITA by flow cytometry. Also, base conversion is measured by targeted PCR amplification and Sanger sequencing for TRAC and CIITA. Multi-antibody panel is used to ascertain multiplex KO level within the CAR+population by flow cytometry.

The technology may generate iPSC lines with both the inclusion of a transgene, in a very specific locus, and multiplex editing at the same time, which may be superior to the current technology available. These edited iPSCs can then be utilised to differentiate, or forward program, into clinically relevant iPSC derived allogeneic CAR T cells.

### Example 5. Generation of Improved NK-Cells for Allogeneic CAR Therapy by CRISPR-Aptamer Based Gene Editing System

In this example, NK-cells are used to prove the utility of the CRISPR based gene editing method for specific integration in the CISH locus of a CAR and simultaneous knock-out of PD1 and NKG2A. With this system, base editing guided knock-out is achieved through recruitment of the deaminase on the target site by sgRNA-aptamer. CAR integration and consequent CISH knock-out is achieved through the same enzyme used for the CRISPR system and for the base editing system. The advantage over previous systems is that one enzyme, or single RNA guided nickase, is used to achieve both the modifications.

The NK-cells are electroporated with mRNA components for both the deaminase-MCP, nCas9-UGI-UGI components, two sgRNAs targeting opposite strands in the first exon of the CISH gene, and sgRNA-aptamer for two different genes (PD1 and NKG2A). The two single nicks generated by the nCas9-UGU-UGI at the two target loci recognized by the sgRNAs result in a staggered double strand break (DSB). After electroporation, cells are transduced with an AAV6 virus used to promote integration of a CAR coding sequence in frame with the CISH gene by homologous directed repair (HDR). Integration of the transgene by HDR or non-homologous end joining (NHEJ) induced by the DSB at this locus results in efficient knock-out of the CISH gene. After transduction, the cells are incubated for 4-7 days and are then checked for CAR expression and knock-out of CISH, PD1 and NKG2A by flow cytometry. Also, base conversion is measured by targeted PCR amplification and Sanger sequencing for PD1 and NKG2A. Multi-antibody panel is used to ascertain multiplex KO level within the CAR+population by flow cytometry.

Thus, the technology may generate NK cells with both the inclusion of a transgene, in a very specific locus, and multiplex editing at the same time, which may be superior to the current technology available. These edited NK cells can be used as improved CAR-NK cells.

### Example 6. Generation of Universal CAR-T Cells by the CRISPR-Aptamer Based Gene Editing System with Knock-Out of 4 Genes

In this example, primary human Pan T lymphocytes were used to prove the utility of the CRISPR based gene editing system for specific integration in the TRAC locus of a CAR gene and simultaneous knock-out of TRAC, B2M, CD52 and PDCD1 genes by the cytosine base editing system. With the method of the present disclosure, base editing guided knock-out was achieved through recruitment of the deaminase on the target site by a sgRNA-aptamer. CAR gene integration and consequent TRAC knock-out was achieved through the same enzyme used for the CRISPR system combined to sgRNAs. The advantage over previous systems is that one CRISPR enzyme, or single RNA guided nickase, is used to achieve both the modifications.

The Pan T cells were activated utilizing anti-CD3 and anti-CD28 antibodies and then electroporated with mRNA components for the following components: the deaminase-MCP, nCas9-UGI-UGI protein, two sgRNAs targeting opposite strands in the first exon of the TRAC gene and sgRNA-aptamer for three different genes. The two single nicks generated by the nCas9-UGU-UGI at the two target loci recognized by the sgRNAs result in a staggered double strand break (DSB). After electroporation, cells were transduced with an AAV6 virus used to promote integration of a CAR coding sequence in frame with the TRAC gene by homologous directed repair (HDR). Integration of the transgene by HDR or non-homologous end joining (NHEJ) induced by the DSB at this locus resulted in efficient knock-out of the TRAC gene and disruption of the TCR complex. After transduction, the cells were incubated for 4-7 days and were then checked for CAR expression and surface knock-out of TCRα/b, B2M, CD52 and PD1 by flow cytometry. Base conversion was measured by targeted PCR amplification and Sanger sequencing.

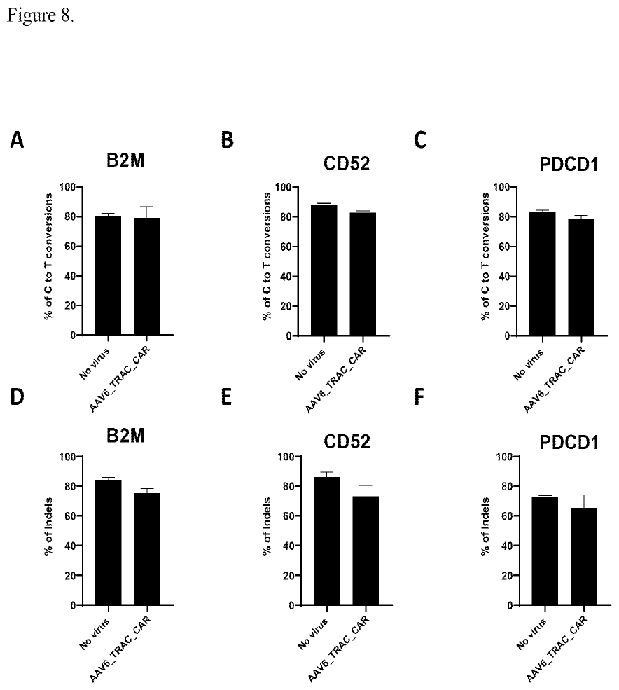

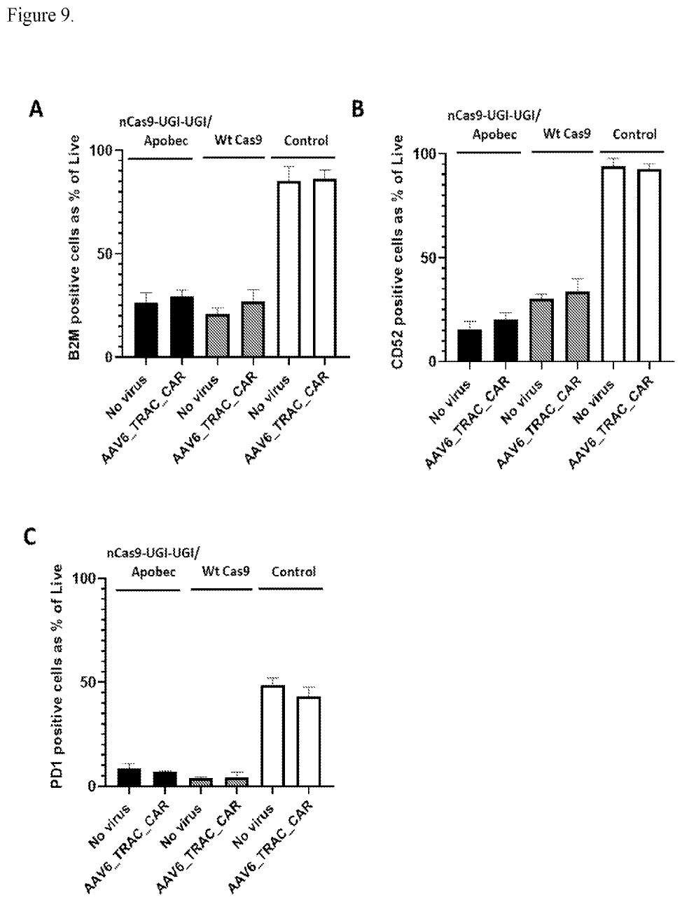

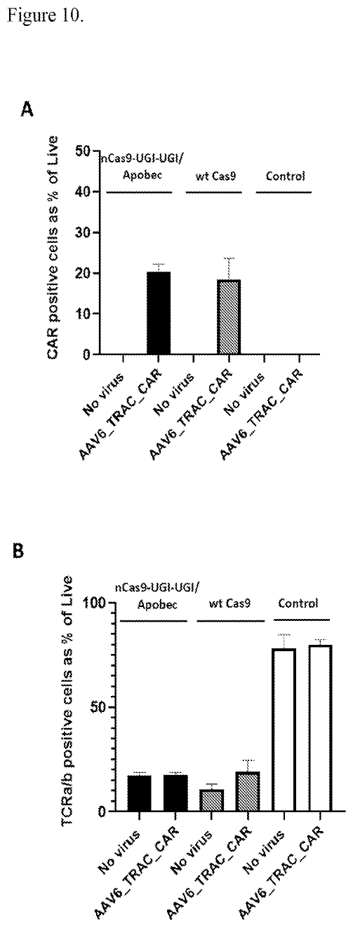

When base editing components were delivered to the cells, high levels of C to T conversions were observed at the three targeted loci, B2M, CD52 and PDCD1, and base editing efficiency was not compromised by the viral vector delivery (FIG. 8A-C). In comparison, similar levels of editing by indel formation were observed with wildtype Cas9 at the three targeted loci, B2M, CD52 and PDCD1 (FIG. 8 D-F). Functional KO for B2M, CD52 and PD1 genes generated by flow cytometry correlated with the base conversion or indel formation and was comparable with the knock-out generated by wt Cas9 (FIG. 9A-C). CAR integration, measured as CAR positive cells or TCR a/b positive cells, was efficiently achieved with the present base editing system and is comparable to the level observed with wild type Cas9 (FIG. 10A-B).

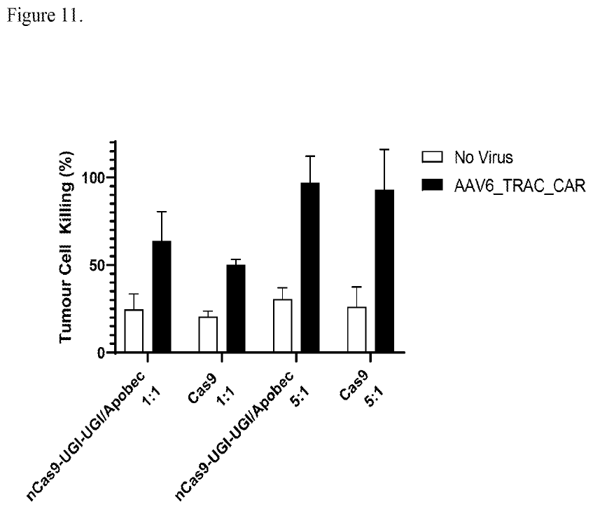

Allogeneic CAR-T cells were generated with the base editing system of the disclosure. For the generation of CAR-T cells, a pair of synthetic sgRNAs targeting exon 1 of the TRAC locus, sgRNA-aptamers for base editing targeting of B2M, CD52, and PDCD1 and nCas9-UGI-UGI and Apobec1-MCP mRNAs were co-delivered into CD3 positive T-cells. Cas9 samples were electroporated with wildtype Cas9 mRNA and regular sgRNAs. This was followed by transduction with the viral vector AAV6-TRAC-CAR. Around 7 days post electroporation, CD3+ cells were depleted from the culture and the resulting CAR-T cells were incubated with CD19 positive Raji cells, previously loaded with Calcein AM, for 4 hours at 1:1 and 5:1 CAR-T:Raji cells ratio. As shown in FIG. 11, the allogeneic CAR-T cells of the disclosure killed efficiently antigen positive cancer cells (CD19 positive Raji cells), and was comparable to the wtCas9 outcome. This data shows that the technology can generate CAR-T cells with functional knock-out in multiple genes that efficiently work as universal CAR-T cells.

### Materials and Methods

### Base Editing Guide RNAs:

Internally generated data was used to specify base editing windows calculated at set distances from the PAM motif (NGG). The data was used to develop algorithms to predict Phenotype or Gene KO applicable guides sequence for the following genes: TRAC, TRBC1, TRBC2, PDCD-1, B2M, and CD52 (Table 4). The sgRNAs were designed including the 1×MS2 aptamer. The guide RNA sequences were synthesized by Horizon Discovery (formerly Dharmacon) and Agilent.

Synthetic 1xMS2 sgRNA sequence
(SEQ ID NO: 32)
mN*mN*NNNNNNNNNNNNNNNNNNGUUUUAGAGCUAGAAAUAGCAAGUU
AAAAUAAGGCUAGUCCGUUAUCAACUUGAAAAAGUGGCACCGAGUCGGU
GCGCGCACAUGAGGAUCACCCAUGUGCUUUUmU*mU*U
(m) 2-O Methyl and (*) phosphorothioate modified
residues

### Knock-In Guide RNAs:

To disrupt the TRAC locus and place the CD19-specific 1928z CAR gene under its transcriptional control, we designed sgRNA pairs targeting the 5′ end of the first exon of TRAC and an AAV vector with homology arms to the target locus and encoding a self-cleaving P2A peptide followed by the CAR cDNA. The sgRNAs to target the TRAC locus were designed following the rule of the PAM-out configuration (PAM sites faced the outside of the target region), with the cleavage sites 40-70 bp apart. The knock-in guides were designed without the 1×MS2 aptamer. The sgRNAs were synthesized by Horizon Discovery (formerly Dharmacon).

Synthetic sgRNA sequences
(SEQ ID NO: 29)
mN*mN*NNNNNNNNNNNNNNNNNNGUUUUAGAGCUAGAAAUAGCAAGUU
AAAAUAAGGCUAGUCCGUUAUCAACUUGAAAAAGUGGCACCGAGUCGGU
GCUmU*mU*U
(m) 2-O Methyl and (*) phosphorothioate modified
residues
5′ sgRNA sequence
(SEQ ID NO: 30)
mG*mA*GAAUCAAAAUCGGUGAAUGUUUUAGAGCUAGAAAUAGCAAGUU
AAAAUAAGGCUAGUCCGUUAUCAACUUGAAAAAGUGGCACCGAGUCGGU
GCUmU*mU*U
3′ sgRNA sequence
(SEQ ID NO: 31)
mA*mA*CAAAUGUGUCACAAAGUAGUUUUAGAGCUAGAAAUAGCAAGUU
AAAAUAAGGCUAGUCCGUUAUCAACUUGAAAAAGUGGCACCGAGUCGGU
GCUmU*mU*U

### Messenger RNA

Messenger RNA molecules were custom synthesized by TriLink Biotechnologies utilising the modified nucleotides pseudouridine and 5-Methyl-Cytosine. The mRNA components translated to the following proteins: Deaminase Apobec 1=NLS-rApobec1-Linker-MCP and nCas9=NLS-nCas9-UGI-UGI-NLS.

### AAV Plasmid Construction

The AAV plasmids were custom synthesized by GenScript. Based on a pAAV backbone we designed the pAAV-TRAC-1928Z_CAR containing in order: 0.9 kb left homology arm of genomic TRAC flanking the 5′ gRNA targeting sequence, a GSG (gly-ser-gly) peptide followed by a self-cleaving P2A peptide in frame with the first exon of TRAC, the 1928z CAR used in Yescarta™ therapy, the bovine growth hormone polyA signal (bGHpA) and 0.9kb right homology arm of the genomic TRAC flanking the 3′ gRNA targeting sequence. Briefly the CD19-CAR (Kochenderfer et al. 2009, J Immunotherapy) comprises a single chain variable fragment scFV specific for the human CD19 derived from the FMC63 mouse hybridoma (Nicholson et al. 1997, Mol Immunology) a portion of the human CD28 molecule (a hinge extracellular part, a transmembrane domain and the entire intracellular domain) and the entire domain of CD3-zeta chain. A second AAV vector has been designed and cloned where the CD19-CAR CDS is replaced by the turboGFP coding sequence.

### Cells

CD3+ T cells were either isolated from whole blood or outsourced from Hemacare. Briefly, peripheral blood mononuclear cells were isolated by density gradient centrifugation (SepMate PBMC isolation tube, STEMCELL Technologies) and T lymphocytes were then purified using the EasySep™ Human T Cell Isolation Kit (STEMCELL Technologies). Cells were activated with Dynabeads (1:1 beads:cell) Human T-Activator CD3/CD28 (ThermoFisher) in Immunocult XT T Cell Expansion medium (STEMCELL Technologies) supplemented with 100 U/ml IL-2 (STEMCELLTechnologies) and 1× Penicillin/Streptomycin (Thermofisher) at 37° C. and 5% CO₂ for 48 hours at a density of 10⁶ cells per ml. Post-activation, beads were removed by placement on a magnet and the cells were transferred back into culture.

### T Cell Electroporation

After 48-72 hours post-activation T cells were electroporated using the Neon Electroporator (Thermofisher). Neon Electroporator conditions were 1600v/10 ms/3 pulses with a 10 μl tip with 250 k cells, combined total of mRNA amount of 1-5 μg, for both the Deaminase-MCP and nCas9-UGI-UGI, and where applicable 2 μM each of the targeting gRNAs. Post-electroporation cells were transferred to Immunocult XT media with 100 U/ml IL-2, 100 U/ml IL-7 and 100 U/ml IL-15 (STEMCELLTechnologies) and cultured at 37° C. and 5% CO₂ for 48-72 hours.

### T Cell Transduction

Recombinant AAV6 particles were generated by Vigene Biosciences. Where applicable, recombinant AAV6 particles were added to the culture 2 to 4 h after electroporation, at the 1×10⁶ GC per cell. Subsequently, edited cells were cultured at 37° C. and 5% CO₂ for 96 hours, maintaining the density of ˜1×10⁶ cells per ml.

### Flow Cytometry

For the detection of PD1 by flow cytometry, cells were stimulated with PMA (50 ng/ml) and ionomycin (250 ng/ml) for 48 hours before the analysis.

T cell identity and QC was confirmed by CD3 antibody staining (Biolegend). T cell activation was confirmed by CD25 staining. CD19-CAR+ cells were detected using an Anti-FMC63 scFv Antibody (AcroBiosystem) at 96 hours post electroporation/transduction. Phenotypic Gene Multiplex KO was assessed at 96 hours post electroporation/transduction: TRAC was confirmed by TCRab antibody staining (Biolegend), B2M by B2M-Antibody (Biolegend), CD52 with a CD52-antibody (Biolegend) and PD1 with a PD1-antibody (Biolegend); any phenotype data was reported as percentage of viable cells, as ascertained by viability dye staining.

### Genomic DNA Analysis

Genomic DNA was released from lysed cells 96 hours post-electroporation. Loci of interest were amplified by PCR and products then sent for Sanger sequencing (Genewiz). Data were analyzed by proprietary in-house software.

### Killing Assay

To test the functionality of CAR-T cells generated with the base editing technology, modified CAR-T cells were firstly depleted of CD3 positive cells using the EasySep™ Human CD3 Positive Selection Kit II (Stemcell) and then incubated with CD19 positive Raji cells at a ratio of 1:1 or 5:1 CAR-T:Raji cells. Before incubation, Raji cells were loaded with Calcein AM. After 4 h of incubation, supernatant from the culture has been collected and analyzed for fluorescent emission using a plate reader with Excitation/Emission of 494/517. The level of fluorescence is proportional to the level of killing of the target Raji cells. The percentage of target cell killing is calculated as [(average of test condition−average of negative control condition)/(average of positive control condition−average of negative control condition)]*100, where negative control condition is Raji cells without CAR-T cells and positive control condition is Raji cells exposed to 2% triton to achieve complete lysis.

### Example 7. Integration of a Promoter-Less Transgene in the B2M Locus of iPSCs with Expression of the Transgene Driven by the B2M Endogenous Promoter and Destruction of B2M Expression and Base Editing of the CIITA Gene

In this example, iPSCs were used to prove the utility of the CRISPR-aptamer based gene editing system for specific integration in the B2M locus of a promoter-less transgene (GFP) and simultaneous knock-out of B2M and CIITA genes by the cytosine base editing system. With the method of the present disclosure, base editing guided knock-out was achieved through recruitment of the deaminase to the target site by a sgRNA-aptamer. GFP integration and consequent B2M knock-out was achieved through the same enzyme used for the CRISPR system, combined with sgRNAs. The advantage over previous systems is that one CRISPR enzyme, or single RNA guided nickase, was used to achieve both modifications, i.e., the same enzyme was used for the transgene knock-in and for the B2M and CIITA genes knock-out.

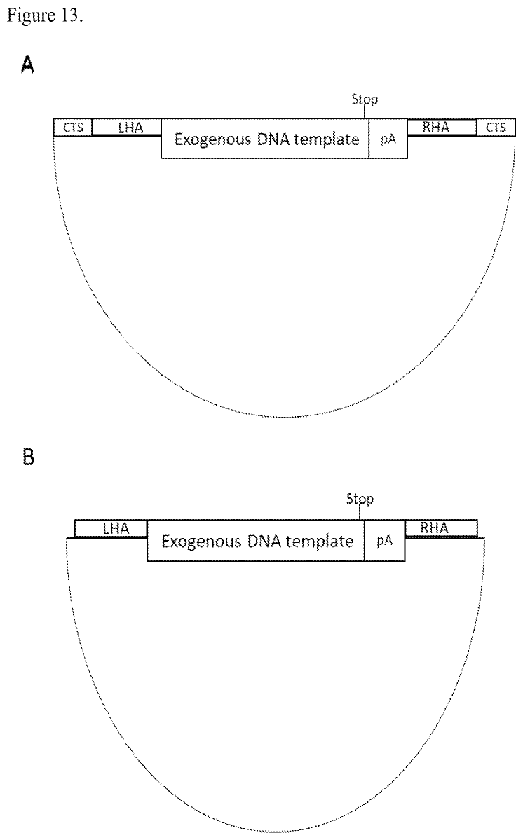

In this example, the exogenous DNA template was delivered in the form of circular or linear double-stranded DNA. The exogenous DNA template was flanked by homology arms from the B2M locus. The exogenous DNA template with homology arms was flanked or not on both sides by sgRNAs B2M targeting sequences (CRISPR/Cas9 target sequences (CTS)) (FIGS. 13 A and B, respectively). In some cases, the GFP transgene flanked by homology arms was flanked by the sequence of the gRNA pair that target the B2M locus, so that once the circular double-stranded DNA is co-delivered in the cells together to the CRISPR components, the donor nucleic acid sequence was released as linear DNA from the circular dsDNA following the cut by CRISPR/Cas.

iPSCs were electroporated with the following components: (i) mRNA encoding the deaminase-MCP (SEQ ID NO: XX), (ii) mRNA encoding nCas9-UGI-UGI protein, (iii) two sgRNAs targeting opposite strands in the first exon of the B2M gene, (iv) sgRNA-aptamer for CIITA gene and (v) circular or linear double-stranded DNA containing the GFP coding sequence with homology arms to the B2M gene. In the circular and linear forms, the homology arms can be flanked (CTS_B2M_tGFP—SEQ ID NO: 136) or not (B2M_tGFP—SEQ ID NO: 135) by sgRNAs B2M targeting sequences (CRISPR/Cas9 target sequences (CTS)).

B2M-tGFP-SEQ ID NO: 135:
CCGTGGCCTTAGCTGTGCTCGCGCTACTCTCTCTTTCTGGCCTGGAGGTAATTCATTCATTCATCCATC
CATTCGTTCATTCGGTTTACTGAGTACCTACTATGTGCCAGCCCCTGTTCTAGGGTGGAAACTAAGAG
AATGATGTACCTAGAGGGCGCTGGAAGCTCTAAAGCCCTAGCAGTTACTGCTTTTACTATTAGTGGT
CGTTTTTTTCTCCCCCCCGCCCCCCGACAAATCAACAGAACAAAGAAAATTACCTAAACAGCAAGGAC
ATAGGGAGGAACTTCTTGGCACAGAACTTTCCAAACACTTTTTCCTGAAGGGATACAAGAAGCAAGA
AAGGTACTCTTTCACTAGGACCTTCTCTGAGCTGTCCTCAGGATGCTTTTGGGACTATTTTTCTTACCC
AGAGAATGGAGAAACCCTGCAGGGAATTCCCAAGCTGTAGTTATAAACAGAAGTTCTCCTTCTGCTA
GGTAGCATTCAAAGATCTTAATCTTCTGGGTTTCCGTTTTCTCGAATGAAAAATGCAGGTCCGAGCA
GTTAACTGGCTGGGGCACCATTAGCAAGTCACTTAGCATCTCTGGGGCCAGTCTGCAAAGCGAGGG
GGCAGCCTTAATGTGCCTCCAGCCTGAAGTCCTAGAATGAGCGCCCGGTGTCCCAAGCTGGGGCGC
GCACCCCAGATCGGAGGGCGCCGATGTACAGACAGCAAACTCACCCAGTCTAGTGCATGCCTTCTTA
AACATCACGAGACTCTAAGAAAAGGAAACTGAAAACGGGAAAGTCCCTCTCTCTAACCTGGCACTG
CGTCGCTGGCTTGGAGACAGGTGACGGTCCCTGCGGGCCTTGTCCTGATTGGCTGGGCACGCGTTT
AATATAAGTGGAGGCGTCGCGCTGGCGGGCATTCCTGAAGCTGACAGCATTCGGGACGAGatggaga
gcgacgagagcggcctgcccgccatggagatcgagtgccgcatcaccggcaccctgaacggcgtggagttcgagctggtgggcgg
cggagagggcacccccgagcagggccgcatgaccaacaagatgaagagcaccaaaggcgccctgaccttcagcccctacctgct
gagccacgtgatgggctacggcttctaccacttcggcacctaccccagcggctacgagaaccccttcctgcacgccatcaacaacgg
cggctacaccaacacccgcatcgagaagtacgaggacggcggcgtgctgcacgtgagcttcagctaccgctacgaggccggccgc
gtgatcggcgacttcaaggtgatgggcaccggcttccccgaggacagcgtgatcttcaccgacaagatcatccgcagcaacgccac
cgtggagcacctgcaccccatgggcgataacgatctggatggcagcttcacccgcaccttcagcctgcgcgacggcggctactaca
gctccgtggtggacagccacatgcacttcaagagcgccatccaccccagcatcctgcagaacgggggccccatgttcgccttccgcc
gcgtggaggaggatcacagcaacaccgagctgggcatcgtggagtaccagcacgccttcaagaccccggatgcagatgccggtga
agaatgactgtgccttctagttgccagccatctgttgtttgcccctcccccgtgccttccttgaccctggaaggtgccactcccactgtc
ctttcctaataaaatgaggaaattgcatcgcattgtctgagtaggtgtcattctattctggggggtggggtggggcaggacagcaag
ggggaggattgggaagacaatagcaggcatgctggggatgcggtgggctctatggGGAGGCTATCCAGCGTGAGTCTC
TCCTACCCTCCCGCTCTGGTCCTTCCTCTCCCGCTCTGCACCCTCTGTGGCCCTCGCTGTGCTCTCTCG
CTCCGTGACTTCCCTTCTCCAAGTTCTCCTTGGTGGCCCGCCGTGGGGCTAGTCCAGGGCTGGATCTC
GGGGAAGCGGCGGGGTGGCCTGGGAGTGGGGAAGGGGGTGCGCACCCGGGACGCGCGCTACTTG
CCCCTTTCGGCGGGGAGCAGGGGAGACCTTTGGCCTACGGCGACGGGAGGGTCGGGACAAAGTTT
AGGGCGTCGATAAGCGTCAGAGCGCCGAGGTTGGGGGAGGGTTTCTCTTCCGCTCTTTCGCGGGGC
CTCTGGCTCCCCCAGCGCAGCTGGAGTGGGGGACGGGTAGGCTCGTCCCAAAGGCGCGGCGCTGA
GGTTTGTGAACGCGTGGAGGGGCGCTTGGGGTCTGGGGGAGGCGTCGCCCGGGTAAGCCTGTCTG
CTGCGGCTCTGCTTCCCTTAGACTGGAGAGCTGTGGACTTCGTCTAGGCGCCCGCTAAGTTCGCATG
TCCTAGCACCTCTGGGTCTATGTGGGGCCACACCGTGGGGAGGAAACAGCACGCGACGTTTGTAGA
ATGCTTGGCTGTGATACAAAGCGGTTTCGAATAATTAACTTATTTGTTCCCATCACATGTCACTTTTAA
AAAATTATAAGAACTACCCGTTATTGACATCTTTCTGTGTGCCAAGGACTTTATGTGCTTTGCGTCATT
TAATTTTGAAAACAGTTATCTTCCGCCATAGATAACTACTATGGTTATCTTCTGCCTCTCACAGATGAA
GAAACTAAGGCACCGAGATTTTAAGAAACTTAATTACACAGGGGATAAATGGCAGCAATCGAGATT
GAAGTCAAGCCGTGGCCTTAGCTGTGCTCGCGCTACTCTCTCTTTCTGGCCTGGAGG
CTS_B2M_tGFP-SEQ ID NO: 136:
TAATTCATTCATTCATCCATCCATTCGTTCATTCGGTTTACTGAGTACCTACTATGTGCCAGCCCCTGTT
CTAGGGTGGAAACTAAGAGAATGATGTACCTAGAGGGCGCTGGAAGCTCTAAAGCCCTAGCAGTTA
CTGCTTTTACTATTAGTGGTCGTTTTTTTCTCCCCCCCGCCCCCCGACAAATCAACAGAACAAAGAAA
ATTACCTAAACAGCAAGGACATAGGGAGGAACTTCTTGGCACAGAACTTTCCAAACACTTTTTCCTG
AAGGGATACAAGAAGCAAGAAAGGTACTCTTTCACTAGGACCTTCTCTGAGCTGTCCTCAGGATGCT
TTTGGGACTATTTTTCTTACCCAGAGAATGGAGAAACCCTGCAGGGAATTCCCAAGCTGTAGTTATA
AACAGAAGTTCTCCTTCTGCTAGGTAGCATTCAAAGATCTTAATCTTCTGGGTTTCCGTTTTCTCGAAT
GAAAAATGCAGGTCCGAGCAGTTAACTGGCTGGGGCACCATTAGCAAGTCACTTAGCATCTCTGGG
GCCAGTCTGCAAAGCGAGGGGGCAGCCTTAATGTGCCTCCAGCCTGAAGTCCTAGAATGAGCGCCC
GGTGTCCCAAGCTGGGGCGCGCACCCCAGATCGGAGGGCGCCGATGTACAGACAGCAAACTCACCC
AGTCTAGTGCATGCCTTCTTAAACATCACGAGACTCTAAGAAAAGGAAACTGAAAACGGGAAAGTC
CCTCTCTCTAACCTGGCACTGCGTCGCTGGCTTGGAGACAGGTGACGGTCCCTGCGGGCCTTGTCCT
GATTGGCTGGGCACGCGTTTAATATAAGTGGAGGCGTCGCGCTGGCGGGCATTCCTGAAGCTGACA
GCATTCGGGACGAGatggagagcgacgagagcggcctgcccgccatggagatcgagtgccgcatcaccggcaccctgaac
ggcgtggagttcgagctggtgggcggcggagagggcacccccgagcagggccgcatgaccaacaagatgaagagcaccaaagg
cgccctgaccttcagcccctacctgctgagccacgtgatgggctacggcttctaccacttcggcacctaccccagcggctacgagaa
ccccttcctgcacgccatcaacaacggcggctacaccaacacccgcatcgagaagtacgaggacggggcgtgctgcacgtgagc
ttcagctaccgctacgaggccggccgcgtgatcggcgacttcaaggtgatgggcaccggcttccccgaggacagcgtgatcttcacc
gacaagatcatccgcagcaacgccaccgtggagcacctgcaccccatgggcgataacgatctggatggcagcttcacccgcacctt
cagcctgcgcgacggcggctactacagctccgtggtggacagccacatgcacttcaagagcgccatccaccccagcatcctgcaga
acgggggccccatgttcgccttccgccgcgtggaggaggatcacagcaacaccgagctgggcatcgtggagtaccagcacgccttc
aagaccccggatgcagatgccggtgaagaatgactgtgccttctagttgccagccatctgttgtttgcccctcccccgtgccttccttg
accctggaaggtgccactcccactgtcctttcctaataaaatgaggaaattgcatcgcattgtctgagtaggtgtcattctattctggg
gggtggggtggggcaggacagcaagggggaggattgggaagacaatagcaggcatgctggggatgcggtgggctctatggGGA
GGCTATCCAGCGTGAGTCTCTCCTACCCTCCCGCTCTGGTCCTTCCTCTCCCGCTCTGCACCCTCTGTG
GCCCTCGCTGTGCTCTCTCGCTCCGTGACTTCCCTTCTCCAAGTTCTCCTTGGTGGCCCGCCGTGGGG
CTAGTCCAGGGCTGGATCTCGGGGAAGCGGCGGGGTGGCCTGGGAGTGGGGAAGGGGGTGCGCA
CCCGGGACGCGCGCTACTTGCCCCTTTCGGCGGGGAGCAGGGGAGACCTTTGGCCTACGGCGACG
GGAGGGTCGGGACAAAGTTTAGGGCGTCGATAAGCGTCAGAGCGCCGAGGTTGGGGGAGGGTTT
CTCTTCCGCTCTTTCGCGGGGCCTCTGGCTCCCCCAGCGCAGCTGGAGTGGGGGACGGGTAGGCTC
GTCCCAAAGGCGCGGCGCTGAGGTTTGTGAACGCGTGGAGGGGCGCTTGGGGTCTGGGGGAGGC
GTCGCCCGGGTAAGCCTGTCTGCTGCGGCTCTGCTTCCCTTAGACTGGAGAGCTGTGGACTTCGTCT
AGGCGCCCGCTAAGTTCGCATGTCCTAGCACCTCTGGGTCTATGTGGGGCCACACCGTGGGGAGGA
AACAGCACGCGACGTTTGTAGAATGCTTGGCTGTGATACAAAGCGGTTTCGAATAATTAACTTATTT
GTTCCCATCACATGTCACTTTTAAAAAATTATAAGAACTACCCGTTATTGACATCTTTCTGTGTGCCAA
GGACTTTATGTGCTTTGCGTCATTTAATTTTGAAAACAGTTATCTTCCGCCATAGATAACTACTATGGT
TATCTTCTGCCTCTCACAGATGAAGAAACTAAGGCACCGAGATTTTAAGAAACTTAATTACACAGGG
GATAAATGGCAGCAATCGAGATTGAAGTCAAG

The two single nicks generated by the nCas9-UGU-UGI at the two target loci recognised by the sgRNAs in B2M exon 1 resulted in staggered double strand breaks (DSB). Integration of the GFP transgene was promoted by the homology arms to the B2M locus by homologous directed repair (HDR). Integration of the transgene by HDR or non-homologous end joining (NHEJ) induced by the DSB at this locus resulted in efficient knock-out of the B2M gene. B2M expression level was low in pluripotent stem cell including iPSCs and it can be induced by treatment with interferon-y. To detect B2M functional knock-out and successful GFP knock-in in the B2M locus, two or four days after electroporation edited cells were treated for 48h with interferon-y and then analyzed by flow cytometry. Base conversion at the CIITA locus was measured by targeted PCR amplification and Sanger sequencing 4-6 days post electroporation.

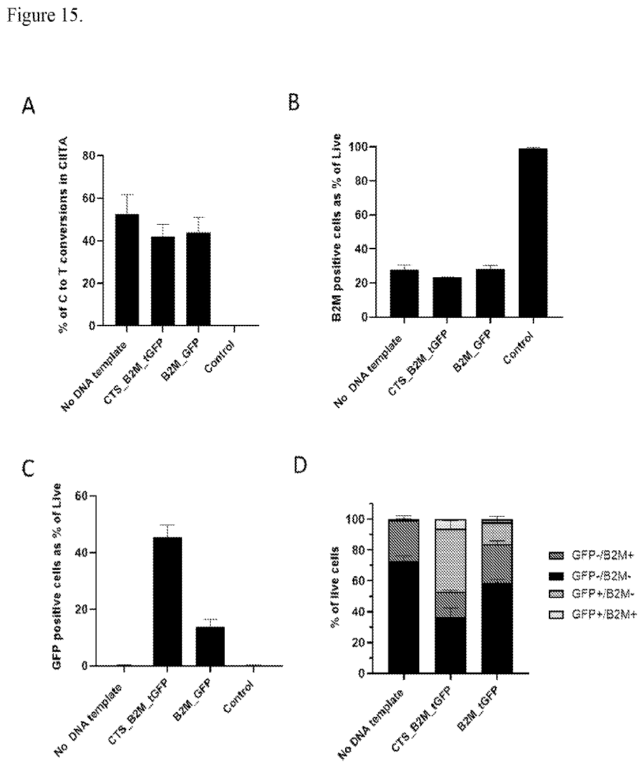

When base editing components and circular double-stranded DNA containing the tGFP coding sequence with homology arms to the B2M were delivered to the cells, C to T conversion was observed at the targeted CIITA locus and base editing efficiency was not compromised by the delivery of the donor DNA (FIG. 15A). Efficient B2M knock-out was observed (FIG. 15B). tGFP integration in the B2M locus, measured as tGFP positive cells in interferon-γ treated cells, was achieved (FIG. 15C). Highest integration was achieved with the donor template flanked on both sides by sgRNAs B2M targeting sequences (CTS_B2M_tGFP). As expected, the majority of GFP positive cells are B2M negative (FIG. 15D).

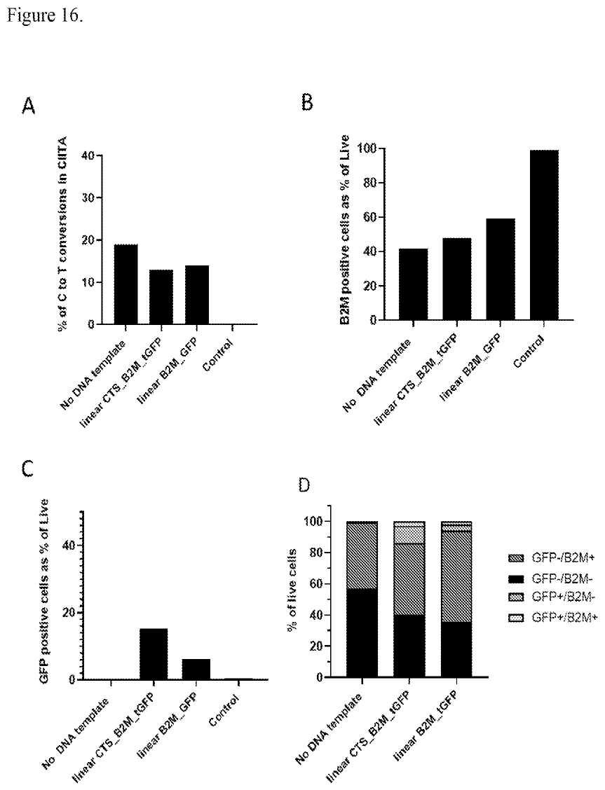

When base editing components and linear double-stranded DNA containing the tGFP coding sequence with homology arms to the B2M were delivered to the cells, C to T conversion was observed at the targeted CIITA locus and base editing efficiency was not compromised by the delivery of the donor DNA (FIG. 16A). Efficient B2M knock-out was observed (FIG. 16B). tGFP integration in the B2M locus, measured as tGFP positive cells in interferon-γ treated cells, was achieved (FIG. 16C). Highest integration was achieved with the donor template flanked on both sides by sgRNAs B2M targeting sequences (CTS_B2M_tGFP). As expected, the majority of GFP positive cells are B2M negative (FIG. 16D).

### Example 8. Generation of Universal iPSCs by the CRISPR-Aptamer Based Gene Editing System

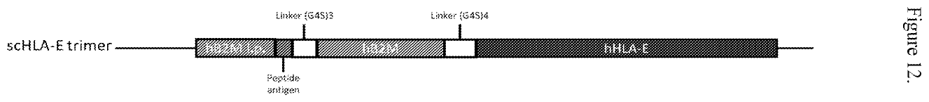

In this example, iPSCs were used to prove the utility of the CRISPR-aptamer based gene editing system for specific integration in the B2M locus of a scHLA-E trimer transgene (See FIG. 12 for the schematic of the construct) and simultaneous knock-out of B2M and CIITA genes by the cytosine base editing system. With the method of the present disclosure, base editing guided knock-out was achieved through recruitment of the deaminase to the target site by a sgRNA-aptamer. scHLA-E trimer integration and consequent B2M knock-out was achieved through the same enzyme used for the CRISPR system, combined with sgRNAs. The advantage over previous systems is that one CRISPR enzyme, or single RNA guided nickase, was used to achieve both the modifications, i.e., the same enzyme was used for the scHLA-E trimer transgene knock-in and for the B2M and CIITA genes knock-out.

In this example, the exogenous DNA template was delivered in the form of circular double-stranded DNA. The exogenous DNA template was flanked by homology arms from the B2M locus, and was flanked or not on both sides by sgRNAs B2M targeting sequences (FIG. 13). In some cases, the scHLA-E trimer transgene flanked by homology arms was flanked by the sequence of the gRNA pair that target the B2M locus, so that once the circular double-stranded DNA is co-delivered in the cells together to the CRISPR components, the donor nucleic acid sequence is released as linear DNA from the plasmid following cut by CRISPR/Cas.

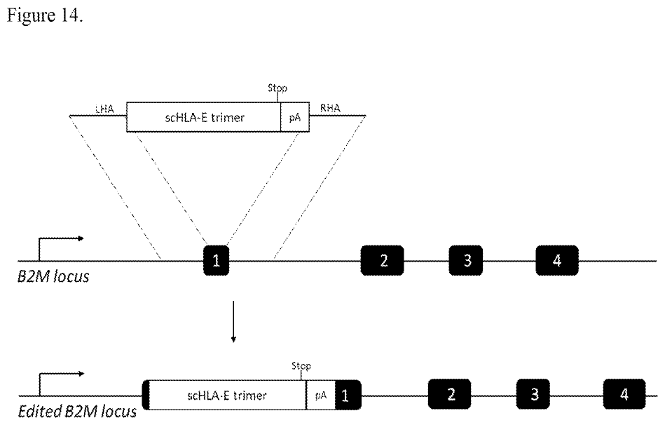

FIG. 14 shows a schematic diagram of an example of a suitable scHLA-E trimer delivery strategy. In this example, the scHLA-E trimer gene was integrated into exon 1 of the B2M locus in frame with the upstream B2M locus. The exogenous DNA template contained the scHLA-E trimer coding sequence flanked by homology sequences (LHA and RHA). Once integrated, scHLA-E trimer expression was driven by the endogenous B2M promoter while the B2M locus was disrupted.

iPSCs were electroporated with the following components: (i) mRNA encoding the deaminase-MCP, (ii) mRNA encoding nCas9-UGI-UGI protein, (iii) two sgRNAs targeting opposite strands in the first exon of the B2M gene, (iv) sgRNA-aptamer for CIITA gene and (v) circular double-stranded exogenous DNA template containing the scHLA-E trimer coding sequence. The two single nicks generated by the nCas9-UGU-UGI at the two target loci recognized by the sgRNAs resulted in staggered double strand breaks (DSB). Integration of the scHLA-E trimer transgene was promoted by the homology arms to the B2M locus by homologous directed repair (HDR). Integration of the transgene by HDR or non-homologous end joining (NHEJ) induced by the DSB at this locus resulted in efficient knock-out of the B2M gene. B2M expression level is low in pluripotent stem cell including iPSCs and it can be induced by treatment with interferon-y. To detect B2M functional knock-out and successful scHLA-E trimer knock-in in the B2M locus, two or four days after electroporation edited cells were treated for 48h with interferon-y and then analyzed by flow cytometry. Base conversion at the CIITA locus was measured by targeted PCR amplification and Sanger sequencing 4-6 days post electroporation.

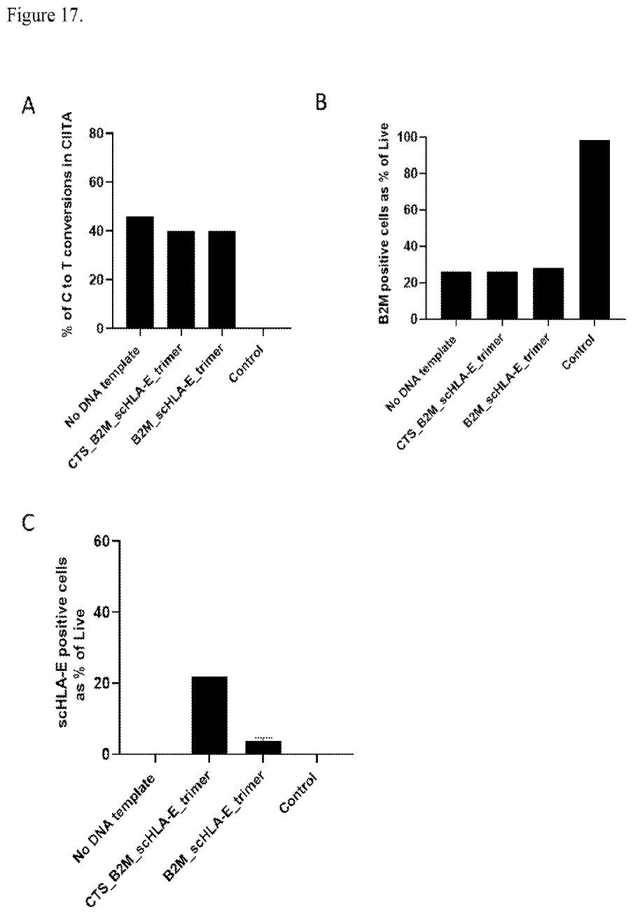

When base editing components and circular double-stranded DNA containing the scHLA-E trimer coding sequence with homology arms to the B2M were delivered to the cells, C to T conversion was observed at the targeted CIITA locus and base editing efficiency was not compromised by the delivery of the donor DNA (FIG. 17A). Efficient B2M knock-out was observed (FIG. 17B). scHLA-E trimer integration in the B2M locus, measured as scHLA-E trimer positive cells in interferon-γ treated cells, was achieved (FIG. 17C). Highest integration was achieved with the donor template flanked on both sides by sgRNAs B2M targeting sequences (CTS_B2M_scHLA-trimer).

This data shows that the present base editing system can efficiently induce site specific integration of a transgene and B2M functional knockout while achieving high level of base editing on another locus (CIITA). These genetic modifications allow the generation of hypoimmunogenic universal iPSCs.

### Materials and Methods for Examples 7 and 8

### Base Editing Guide RNAs:

Internally generated data was used to specify base editing windows calculated at set distances from the PAM motif (NGG). The data was used to develop algorithms to predict Phenotype or Gene KO applicable guides sequence for CIITA (Table X). The sgRNAs were designed, including the 1×MS2 aptamer. The guide RNA sequences were synthesized by Horizon Discovery (formerly Dharmacon) and Agilent.

Synthetic 1xMS2 sgRNA sequence
(SEQ ID NO: 32)
mN*mN*NNNNNNNNNNNNNNNNNNGUUUUAGAGCUAGAAAUAGCAAGUU
AAAAUAAGGCUAGUCCGUUAUCAACUUGAAAAAGUGGCACCGAGUCGGU
GCGCGCACAUGAGGAUCACCCAUGUGCUUUUmU*mU*U
(m) 2-O Methyl and (*) phosphorothioate modified
residues

TABLE 6
crRNA sequences in the single-guide RNAs (sgRNAs) for CIITA genes. An example list
of guide designs for guide RNA sequences that can create a functional knock-out using
the base editing technology exemplified. The list includes guides specific to the
introduction of a premature stop codon and splice disruption sites, which were
generated by an in-house proprietary software.

| Gene | | KO | | | | |
|---|---|---|---|---|---|---|
| Name | Guide ID | Type* | Strand | Guide Sequence | SEQ ID NO: | PAM |
| CIITA | CIITA_1 | Stop | sense | GAGCCCCAAGGTAAAAAGGC | 137 | CGG |
| | CIITA_2 | Stop | sense | AGCCCCAAGGTAAAAAGGCC | 138 | GGG |
| | CIITA_3 | Stop | sense | CAGCTCACAGTGTGCCACCA | 139 | TGG |
| | CIITA_4 | Stop | sense | TATGACCAGATGGACCTGGC | 140 | TGG |
| | CIITA_5 | Stop | sense | ACTGGACCAGTATGTCTTCC | 141 | AGG |
| | CIITA_6 | Stop | sense | TGTCTTCCAGGACTCCCAGC | 142 | TGG |
| | CIITA_7 | Stop | sense | CTTCCAGGACTCCCAGCTGG | 143 | AGG |
| | CIITA_8 | Stop | sense | TTCCAGGACTCCCAGCTGGA | 144 | GGG |
| | CIITA_9 | Stop | anti | TTCCAGTGCTTCAGGTCTGC | 145 | CGG |
| | CIITA_10 | Stop | sense | TTCAACCAGGAGCCAGCCTC | 146 | CGG |
| | CIITA_11 | Stop | sense | GACCAGATTCCCAGTATGTT | 147 | AGG |
| | CIITA_12 | Stop | sense | ACCAGATTCCCAGTATGTTA | 148 | GGG |
| | CIITA_13 | Stop | sense | CTCTGGCAAATCTCTGAGGC | 149 | TGG |
| | CIITA_14 | Stop | sense | AGCCAAGTACCCCCTCCCAG | 150 | TGG |
| | CIITA_15 | Stop | sense | ACCTCCCGAGCAAACATGAC | 151 | AGG |
| | CIITA_16 | Stop | sense | CCCACCCAATGCCCGGCAGC | 152 | TGG |
| | CIITA_17 | Stop | anti | AGGCCATTTTGGAAGCTTGT | 153 | TGG |
| | CIITA_18 | Stop | sense | TGGTGCAGGCCAGGCTGGAG | 154 | AGG |
| | CIITA_19 | Stop | sense | GAACGGCAGCTGGCCCAAGG | 155 | AGG |
| | CIITA_20 | Stop | sense | GGCCCAAGGAGGCCTGGCTG | 156 | AGG |
| | CIITA_21 | Stop | sense | GACACGAGTGATTGCTGTGC | 157 | TGG |
| | CIITA_22 | Stop | sense | ACACGAGTGATTGCTGTGCT | 158 | GGG |
| | CIITA_23 | Stop | sense | CTGGTCAGGGCAAGAGCTAT | 159 | TGG |
| | CIITA_24 | Stop | sense | TGGTCAGGGCAAGAGCTATT | 160 | GGG |
| | CIITA_25 | Stop | sense | TTCCAGAAGAAGCTGCTCCG | 161 | AGG |
| | CIITA_26 | Stop | sense | CAGACATCAAAGTACCCTAC | 162 | AGG |
| | CIITA_27 | Stop | sense | ACATCAAAGTACCCTACAGG | 163 | AGG |
| | CIITA_28 | Stop | anti | CGCCCAGGTCCTCACGTCTG | 164 | CGG |
| | CIITA_29 | Stop | sense | CTTAGTCCAACACCCACCGC | 165 | GGG |
| | CIITA_30 | Stop | sense | GGAAGCAGAAGGTGCTTGCG | 166 | AGG |
| | CIITA_31 | Stop | sense | GGCTGCAGCCGGGGACACTG | 167 | CGG |
| | CIITA_32 | Stop | sense | GCTGCAGCCGGGGACACTGC | 168 | GGG |
| | CIITA_33 | Stop | anti | CTGCCAAATTCCAGCCTCCT | 169 | CGG |
| | CIITA_34 | Stop | sense | GGCGGGCCAAGACTTCTCCC | 170 | TGG |
| | CIITA_35 | Stop | sense | TGTGCAGACTCAGAGGTGAG | 171 | AGG |
| | CIITA_36 | Stop | sense | AGACTCAGAGGTGAGAGGAG | 172 | AGG |
| | CIITA_37 | Stop | sense | CTCAGAGGTGAGAGGAGAGG | 173 | CGG |
| | CIITA_38 | Stop | sense | CGTCCAGTACAACAAGTTCA | 174 | CGG |
| | CIITA_39 | Splice | anti | TTTTACCTTGGGGCTCTGAC | 175 | AGG |
| | CIITA_40 | Splice | anti | TTCTGGGAGGAAAAGTCCCT | 176 | TGG |
| | CIITA_41 | Splice | anti | TACTGAAAATGTCCTTGCTC | 177 | AGG |
| | CIITA_42 | Splice | anti | CACCTGGCTTCCAGTGCTTC | 178 | AGG |
| | CIITA_43 | Splice | anti | GGGCTCAGCTGTGAGGAAGT | 179 | GGG |
| | CIITA_44 | Splice | anti | TAACATACTGGGAATCTGGT | 180 | CGG |
| | CIITA_45 | Splice | anti | CTTACCTGTCATGTTTGCTC | 181 | GGG |
| | CIITA_46 | Splice | anti | CCTTACCTGTCATGTTTGCT | 182 | CGG |
| | CIITA_47 | Splice | anti | TGCTCTGGAGATGGAGAAGC | 183 | AGG |
| | CIITA_48 | Splice | anti | ACCGGCTCTGCAAAGGCCAG | 184 | GGG |

*Stop = Premature stop codon, Splice = Splice site disruption

### Knock-In Guide RNAs:

To disrupt the B32M locus and place the scHLA-E trimer gene under its transcriptional control, we designed sgRNA pairs targeting the 5′ end of the first exon of B32M and a donor DNA template with homology arms to the target locus and encoding scHLA-E trimer. The sgRNAs to target the B32M locus were designed following the rule of the PAM-out configuration (PAM sites faced the outside of the target region), with the cleavage sites 40-70 bp apart (Table 7). The knock-in guides were designed without the 1×MS2 aptamer. The sgRNAs were synthesised by Horizon Discovery (formerly Dharmacon).

Synthetic sgRNA sequences
(SEQ ID NO: 29)
mN*mN*NNNNNNNNNNNNNNNNNNGUUUUAGAGCUAGAAAUAGCAAGUU
AAAAUAAGGCUAGUCCGUUAUCAACUUGAAAAAGUGGCACCGAGUCGGU
GCUmU*mU*U
(m) 2-O Methyl and (*) phosphorothioate modified
residues
5′ sgRNA sequence 2
(SEQ ID NO: 185)
mC*mG*CGAGCACAGCUAAGGCCAGUUUUAGAGCUAGAAAUAGCAAGUU
AAAAUAAGGCUAGUCCGUUAUCAACUUGAAAAAGUGGCACCGAGUCGGU
GCUmU*mU*U
3′ sgRNA sequence 2
(SEQ ID NO: 186)
mA*mC*UCUCUCUUUCUGGCCUGGGUUUUAGAGCUAGAAAUAGCAAGUU
AAAAUAAGGCUAGUCCGUUAUCAACUUGAAAAAGUGGCACCGAGUCGGU
GCUmU*mU*U

TABLE 7
Single guide RNA for knock-in integration in the B2M locus. An example list of guide
designs for guide RNA sequences that, when used as a pair and combined with the nCas9-
UGI-UGI of the Examples, generate two nicks in opposite strands, creating a functional
knock-out and inducing site-specific integration using the base editing technology
exemplified and a template transgene with homology arms to the locus. Single guide
RNA with PAM located 5′ to the integration site have to be combined with single
guide RNA with PAM located 3′ to the integration site.

| | | | PAM location | | | |
|---|---|---|---|---|---|---|
| Gene | | | to the | | SEQ | |
| Name | Guide ID | Strand | integration site | Spacer Sequence | ID: | PAM |
| B2M | B2M_5′_1 | anti | 5′ | GCCCGAATGCTGTCAGCTTC | 187 | AGG |
| | B2M_5′_2 | anti | 5′ | GGCCACGGAGCGAGACATCT | 188 | CGG |
| | B2M_5′_3 | anti | 5′ | CGCGAGCACAGCTAAGGCCA | 189 | CGG |
| | B2M_5′_4 | anti | 5′ | GAGTAGCGCGAGCACAGCTA | 190 | AGG |
| | B2M_3′_1 | sense | 3′ | GGCCGAGATGTCTCGCTCCG | 191 | TGG |
| | B2M_3′_2 | sense | 3′ | CTCGCGCTACTCTCTCTTTC | 192 | TGG |
| | B2M_3′_3 | sense | 3′ | GCTACTCTCTCTTTCTGGCC | 193 | TGG |
| | B2M_3′_4 | sense | 3′ | ACTCTCTCTTTCTGGCCTGG | 194 | AGG |

### Messenger RNA

Messenger RNA molecules were custom synthesized by TriLink Biotechnologies utilizing the modified nucleotides pseudouridine and 5-Methyl-Cytosine. The mRNA components translated to the following proteins: Deaminase Apobec 1=NLS-rApobec1-Linker-MCP and nCas9=NLS-nCas9-UGI-UGI-NLS.

### Donor DNA Template Construction

The donor DNA templates were custom synthesized and cloned in the pUC19 cloning plasmid by GenScript. The donor DNA template coding for the scHLA-E trimer contains in order: 0.9 kb left homology arm of genomic B2M flanking the 5′ gRNA targeting sequence, the scHLA-E trimer coding sequence, the bovine growth hormone polyA signal (bGHpA) and 0.9kb right homology arm of the genomic B2M flanking the 3′ gRNA targeting sequence. Briefly, the scHLA-E trimer is a chimeric protein that comprises the following elements: (a) the leader peptide of B2M, (b) VMAPRTLIL (a HLA-E-binding peptide), (c) a 15 amino acid linker (G4S)3, (d) a mature human B2M, (e) a 20 amino acid linker (G4S)4 and (f) a mature HLA-E heavy chain. The donor DNA template with homology arms is flanked or not on both sides by the sgRNAs targeting sequences that target the B2M locus. A second donor DNA template has been designed and cloned where the scHLA-E trimer coding sequence was replaced by the turboGFP coding sequence. To obtain the linear double-stranded form of the donor DNA templates, the above plasmids were digested with specific restriction enzymes to excise the donor DNA template from the plasmid. The fragment of the right size was then purified after gel electrophoresis using a gel purification kit.

Human iPSC Culture

Frozen human iPSCs were obtained from ThermoFisher Scientific (Gibco line). Cells were thawed and cultured in mTesr-PLUS medium (STEMCELL Technologies) on Vitronectin-XF (STEMCELL Technologies) coated non-adherent cell-culture plasticware (Greiner Bio-One) at 37 C and 5% CO2. When confluent cells were passaged in clumps using Versene dissociation reagent (ThermoFisher Scientific). Medium was exchanged at 1-3 day intervals, and cells were passaged at 3-5 day intervals, as required.

Human iPSC Electroporation and Culture Post Electroporation

2-4 hrs prior to electroporation iPSCs were fed with fresh mTesr-PLUS culture medium (STEMCELL Technologies) containing 10 μM Y-27632 (STEMCELL Technologies), then dissociated to single cells using Accutase (ThermoFisher Scientific). 150 k-200 k cells were resuspended in 20 μl of Buffer P3 (Lonza) and combined with 1-4 μg of modified mRNA (Trilink) encoding Deaminase-MCP and nCas9-UGI proteins, 1-4 μM of each sgRNA (Agilent) and 1-2 ug of donor DNA. Electroporation was performed with the 4D Nucleofector (Lonza) in a 16×20 μl multi-well cuvette using programmes CM138 or DN100. Post-electroporation, cells were seeded on Geltrex (ThermoFisher Scientific) coated cell-culture plasticware (Corning) in mTesr-PLUS (STEMCELL Technologies) with the inclusion of 10 μM of the Rho-kinase inhibitor Y-27632 (STEMCELL Technologies) in culture medium for 24 hrs post-electroporation to promote cell survival. Two- or 4-days post electroporation cells were treated with interferon-y at a concentration of 100 ng/ml in mTesr-PLUS culture medium (STEMCELL Technologies) for 48 h before flow cytometry analysis.

### Flow Cytometry

scHLA-E trimer positive cells were detected using an Anti-HLA-E Antibody (Biolegend) after 48 hours treatment with interferon-y. Phenotypic knock-out of B2M was assessed after 48 hours treatment with interferon-y using a B2M-Antibody (Biolegend); any phenotype data was reported as percentage of viable cells, as ascertained by viability dye staining.

### Genomic DNA Analysis

Genomic DNA was released from lysed cells 96 hours post-electroporation. Loci of interest were amplified by PCR and products then sent for Sanger sequencing (Genewiz). Data were analyzed by proprietary in-house software.

1. A method for making multiple genetic modifications to a cell, the method comprising introducing into the cell and/or expressing in the cell:
a) a CRISPR system for integrating an exogenous sequence at a first target nucleic acid sequence, the CRISPR system comprising:
i) a first gRNA and a second gRNA that are complementary to opposite strands of the first target nucleic acid sequence; and
ii) a donor nucleic acid sequence comprising the exogenous sequence;
b) a base editing system for introducing a genetic modification at a second target nucleic acid sequence, the base editing system comprising:
i) an RNA scaffold comprising (i) a gRNA sequence that is complementary to the second target nucleic acid sequence and, (ii) a recruiting RNA motif; and
ii) an effector fusion protein comprising (i) an RNA binding domain capable of binding to the recruiting RNA motif and (ii) an effector domain comprising a base modifying enzyme; and
c) a RNA guided nickase capable of interacting with the first and second gRNAs of the CRISPR system and with the RNA scaffold of the base editing system; and
d) culturing the cell to produce a cell comprising multiple genetic modifications.

2. The method according to claim 1, wherein the base modifying enzyme has cytosine deamination activity, adenosine deamination activity, DNA methyl transferase activity, or demethylase activity.

3. The method according to claim 1 or 2, wherein the RNA guided nickase is a CRISPR Type II or Type V enzyme.

4. The method according to any one of the preceding claims, wherein the RNA guided nickase is a Cas9 nickase.

5. The method according to any one of the preceding claims, wherein the RNA scaffold comprises a tracrRNA.

6. The method according to any one of the preceding claims, wherein the method uses a modular system comprising multiple base editing systems capable of binding to different target nucleic acid sequences to genetically modify multiple different genetic loci.

7. The method according to any one of the preceding claims, wherein the cell is an immune cell or an hPSC.

8. The method according to claim 7, wherein the hPSC is an iPSC.

9. The method according to claim 7, wherein the cell is an immune cell selected from T cells, Natural Killer cells (NK cell), B cells, myeloblast lymphoblastor CD34+ hematopoietic stem and progenitor cells (HSPC).

10. The method according to any one of the preceding claims, wherein the first gRNA and second gRNA are complementary to opposite strands of a TRAC or B2M or CISH locus.

11. The method according to any one of the preceding claims, wherein the CRISPR system is used to introduce a donor nucleic acid sequence comprising a CAR or a TCR encoding sequence flanked by homology arms specific to the target locus.

12. The method according to claim 11, wherein the CAR or TCR or scHLA-E trimer encoding sequence is integrated at the TRAC or B2M or CISH locus.

13. The method according to claim 12, wherein expression of the CAR or TCR or scHLA-E trimer encoding sequence is driven by an endogenous TRAC or B2M or CISH promoter.

14. The method according to any one of the preceding claims, wherein the multiple genetic modifications occur simultaneously.

15. The method according to any one of the preceding claims, wherein the nucleic acids encoding each of the CRISPR system, base editing system and the RNA guided nickase are introduced into the cell in a single delivery step.

16. The method according to any one of the preceding claims, wherein the donor nucleic acid sequence is introduced into the cell using a viral vector.

17. The method according to claim 16, wherein the viral vector is an AAV.

18. The method according to any one of the preceding claims, wherein the base editing system introduces one or more genetic modifications that correct a genetic mutation, inactivate the expression of a gene, change the expression levels of a gene, or change intron-exon splicing.

19. The method according to any one of the preceding claims, wherein the genetic modification introduced by the base editing system is a point mutation.

20. The method according to claim 19, wherein the point mutation introduces a premature stop codon, disrupts a start codon, disrupts a splice site or corrects a genetic mutation.

21. The method according to any one of the preceding claims, wherein the genetic modification introduced by the base editing system results in reduced expression of any one or more of the genes selected from the group consisting of TRAC, TRBC1, TRBC2, PDCD1, CD52, CIITA, NKG2A and B2M.

22. The method according to any one of the preceding claims, wherein the RNA scaffold is introduced into the cell as chemically synthesized RNA.

23. The method according to any one of the preceding claims, wherein the RNA scaffold comprises one or more chemical modifications.

24. The method according to any one of the preceding claims, wherein the recruiting RNA motif is located at the 3′ end of the RNA scaffold.

25. The method according to any one of the preceding claims, wherein the RNA scaffold comprises two or more recruiting RNA motifs.

26. The method according to any one of the preceding claims, wherein the recruiting RNA motif is an RNA aptamer.

27. The method according to any one of the preceding claims, wherein the recruiting RNA motif is an MS2 aptamer.

28. The method according to any one of the preceding claims, wherein the RNA guided nickase is nCas9 with one or two UGIs and the recruiting RNA motif is a single MS2 aptamer located at the 3′ end of the RNA scaffold.

29. The method according to any one of the preceding claims, wherein the genetic modification introduced results in the generation of an allogeneic T-cell.

30. A genetically modified cell obtained by the method of any one of the preceding claims.

31. A genetically modified cell obtained by the method of any one of claims 1 to 29, comprising an exogenous CAR or TCR encoding sequence in the endogenous TRAC or B2M locus and at least one point mutation in 2 or more genes.

32. The genetically modified cell of claim 31, wherein the 2 or more genes are selected from the group consisting of TRAC, TRBC1, TRBC2, PDCD1, CD52, CIITA, NKG2A and B2M, resulting in the functional knock-out of said genes.

33. A system for genetically modifying a cell comprising:
a) a CRISPR system for integrating an exogenous sequence at a first target nucleic acid sequence, the CRISPR system comprising:
iii) a first gRNA and a second gRNA that are complementary to opposite strands of the first target nucleic acid sequence; and
iv) a donor nucleic acid sequence comprising the exogenous sequence;
b) a base editing system for introducing a genetic modification at a second target nucleic acid sequence, the base editing system comprising:
iii) an RNA scaffold comprising (i) a gRNA sequence that is complementary to the second target nucleic acid sequence and, (ii) a recruiting RNA motif; and
iv) an effector fusion protein comprising (i) an RNA binding domain capable of binding to the recruiting RNA motif and (ii) an effector domain comprising a base modifying enzyme; and
c) an RNA guided nickase capable of interacting with the first and second gRNAs of the CRISPR system and with the RNA scaffold of the base editing system.


SEQUENCE LISTING (195 sequences)

SEQ ID NO: 1 - protein, 9 aa, unidentified
VMAPRTLIL

SEQ ID NO: 2 - DNA, 1524 bp, unidentified
  source 1..1524: mol_type = unassigned DNA
aagctttgagccgagatgtctcgctccgtggccttagctgtgctcgcgctactctctctttctggcctcgaggctgttatggctccgcggactttaattttaggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaatctaga

SEQ ID NO: 3 - DNA, 3535 bp, synthetic construct
  source 1..3535: mol_type = other DNA
taattcattcattcatccatccattcgttcattcggtttactgagtacctactatgtgccagcccctgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggacgagatgtctcgctcagtcgccttagctgtgctcgcgctactctctctgtccgggctcgaagctgttatggctccgcggactttaattttaggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaataaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaag

SEQ ID NO: 4 - DNA, 3631 bp, synthetic construct
  source 1..3631: mol_type = other DNA
ccgtggccttagctgtgctcgcgctactctctctttctggcctggaggtaattcattcattcatccatccattcgttcattcggtttactgagtacctactatgtgccagcccctgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggacgagatgtctcgctcagtcgccttagctgtgctcgcgctactctctctgtccgggctcgaagctgttatggctccgcggactttaattttaggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaataaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagccgtggccttagctgtgctcgcgctactctctctttctggcctggagg

SEQ ID NO: 5 - protein, 84 aa, unidentified
  source 1..84: note = Bacillus Phage PBS2
MTNLSDIIEKETGKQLVIQESILMLPEEVEEVIGNKPESDILVHTAYDESTDENVMLLTSDAPEYKPWALVIQDSNGENKIKML

SEQ ID NO: 6 - RNA, 66 nt, unidentified
  source 1..66: mol_type = other RNA; note = Yeast
ttcttgtcgtacttatagatcgctacgttatttcaattttgaaaatctgagtcctgggagtgcgga

SEQ ID NO: 7 - protein, 609 aa, unidentified
  source 1..609: note = Yeast
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 8 - protein, 485 aa, unidentified
  source 1..485: note = Yeast
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 9 - RNA, 10 nt, unidentified
  source 1..10: mol_type = unassigned RNA; note = Yeast
aatttttgga

SEQ ID NO: 10 - protein, 83 aa, unidentified
  source 1..83: note = Yeast
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 11 - RNA, 25 nt, unidentified
  source 1..25: mol_type = unassigned RNA; note = phage
gcgcacatgaggatcacccatgtgc

SEQ ID NO: 12 - protein, 117 aa, unidentified
  source 1..117: note = phage
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 13 - RNA, 26 nt, unidentified
  source 1..26: mol_type = unassigned RNA; note = phage
ataaggagtttatatggaaaccctta

SEQ ID NO: 14 - protein, 128 aa, unidentified
  source 1..128: note = phage
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 15 - RNA, 19 nt, unidentified
  source 1..19: mol_type = unassigned RNA; note = phage
ctgaatgcctgcgagcatc

SEQ ID NO: 16 - protein, 62 aa, unidentified
  source 1..62: note = phage
MKSIRCKNCNKLLFKADSFDHIEIRCPRCKRHIIMLNACEHPTEKHCGKREKITHSDETVRY

SEQ ID NO: 17 - protein, 229 aa, unidentified [sequence omitted]

SEQ ID NO: 18 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 19 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 20 - DNA, 4101 bp, synthetic construct
  source 1..4101: mol_type = other DNA
gataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgac

SEQ ID NO: 21 - DNA, 177 bp, synthetic construct
  source 1..177: mol_type = other DNA
nnnnnnnnnnnnnnnnnnnngttttagagctagaaatagcaagttaaaataaggctagtccgttatcaacttgaaaaagtggcaccgagtcggtgcgggagcacatgaggatcacccatgtgccacgagcgacatgaggatcacccatgtcgctcgtgttccctttttttctccgct

SEQ ID NO: 22 - DNA, 157 bp, synthetic construct
  source 1..157: mol_type = other DNA
gttttagagctagaaatagcaagttaaaataaggctagtccgttatcaacttgaaaaagtggcaccgagtcggtgcgggagcacatgaggatcacccatgtgccacgagcgacatgaggatcacccatgtcgctcgtgttccctttttttctccgct

SEQ ID NO: 23 - protein, 340 aa, synthetic construct
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 24 - protein, 371 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 25 - DNA, 594 bp, unidentified
  source 1..594: mol_type = unassigned DNA
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactt

SEQ ID NO: 26 - protein, 198 aa, unidentified [sequence omitted]

SEQ ID NO: 27 - DNA, 594 bp, synthetic construct
  source 1..594: mol_type = other DNA
atggacagcctcttgatgaaccggaggaagtttctttaccaattcaaaaatgtccgctgggctaagggtcggcgtgagacctacctgtgctacgtagtgaagaggcgtgacgccgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactt

SEQ ID NO: 28 - protein, 198 aa, synthetic construct
MDSLLMNRRKFLYQFKNVRWAKGRRETYLCYVVKRRDAATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 29 - RNA, 100 nt, synthetic construct
  source 1..100: mol_type = other RNA
  modified_base 1..2: mod_base = OTHER; note = 2-O Methyl nucleotide
  modified_base 98..99: mod_base = um
  modified_base 1^2: mod_base = OTHER; note = Phosphorothioate bond
  modified_base 2^3: mod_base = OTHER; note = Phosphorothioate bond
  modified_base 98^99: mod_base = OTHER; note = Phosphorothioate bond
  modified_base 99^100: mod_base = OTHER; note = Phosphorothioate bond
nnnnnnnnnnnnnnnnnnnngttttagagctagaaatagcaagttaaaataaggctagtccgttatcaacttgaaaaagtggcaccgagtcggtgctttt

SEQ ID NO: 30 - RNA, 100 nt, synthetic construct
  source 1..100: mol_type = other RNA
  modified_base 1^2: mod_base = OTHER; note = Phosphorothioate bond
  modified_base 2^3: mod_base = OTHER; note = Phosphorothioate bond
  modified_base 98^99: mod_base = OTHER; note = Phosphorothioate bond
  modified_base 99^100: mod_base = OTHER; note = Phosphorothioate bond
  modified_base 1: mod_base = gm
  modified_base 98..99: mod_base = um
  modified_base 2: mod_base = OTHER; note = 2-O Methyl adenosine
gagaatcaaaatcggtgaatgttttagagctagaaatagcaagttaaaataaggctagtccgttatcaacttgaaaaagtggcaccgagtcggtgctttt

SEQ ID NO: 31 - RNA, 100 nt, synthetic construct
  source 1..100: mol_type = other RNA
  modified_base 1^2: mod_base = OTHER; note = Phosphorothioate bond
  modified_base 2^3: mod_base = OTHER; note = Phosphorothioate bond
  modified_base 98^99: mod_base = OTHER; note = Phosphorothioate bond
  modified_base 99^100: mod_base = OTHER; note = Phosphorothioate bond
  modified_base 1..2: mod_base = OTHER; note = 2-O Methyl adenosine
  modified_base 98..99: mod_base = um
aacaaatgtgtcacaaagtagttttagagctagaaatagcaagttaaaataaggctagtccgttatcaacttgaaaaagtggcaccgagtcggtgctttt

SEQ ID NO: 32 - RNA, 128 nt, synthetic construct
  source 1..128: mol_type = other RNA
  modified_base 1^2: mod_base = OTHER; note = Phosphorothioate bond
  modified_base 2^3: mod_base = OTHER; note = Phosphorothioate bond
  modified_base 126^127: mod_base = OTHER; note = Phosphorothioate bond
  modified_base 127^128: mod_base = OTHER; note = Phosphorothioate bond
  modified_base 1..2: mod_base = OTHER; note = 2-O Methyl nucleotide
  modified_base 127..128: mod_base = um
nnnnnnnnnnnnnnnnnnnngttttagagctagaaatagcaagttaaaataaggctagtccgttatcaacttgaaaaagtggcaccgagtcggtgcgcgcacatgaggatcacccatgtgcttttttt

SEQ ID NO: 33 - DNA, 20 bp, synthetic construct
  source 1..20: mol_type = other DNA
cacagcccaagatagttaag

SEQ ID NO: 34 - DNA, 20 bp, synthetic construct
  source 1..20: mol_type = other DNA
acagcccaagatagttaagt

SEQ ID NO: 35 - DNA, 20 bp, synthetic construct
  source 1..20: mol_type = other DNA
ttaccccacttaactatctt

SEQ ID NO: 36 - DNA, 20 bp, synthetic construct
  source 1..20: mol_type = other DNA
cttaccccacttaactatct

SEQ ID NO: 37 - DNA, 20 bp, synthetic construct
  source 1..20: mol_type = other DNA
actcacgctggatagcctcc

SEQ ID NO: 38 - DNA, 20 bp, synthetic construct
  source 1..20: mol_type = other DNA
ttggagtacctgaggaatat

SEQ ID NO: 39 - DNA, 20 bp, synthetic construct
  source 1..20: mol_type = other DNA
tcgatctatgaaaaagacag

SEQ ID NO: 40 - DNA, 20 bp, synthetic construct
  source 1..20: mol_type = other DNA
aacctgaaaagaaaagaaaa

SEQ ID NO: 41 - DNA, 20 bp, synthetic construct
  source 1..20: mol_type = other DNA
gtacaggtaagagcaacgcc

SEQ ID NO: 42 - DNA, 20 bp, synthetic construct
  source 1..20: mol_type = other DNA
ctcctcctacagatacaaac

SEQ ID NO: 43 - DNA, 20 bp, synthetic construct
  source 1..20: mol_type = other DNA
cagatacaaactggactctc

SEQ ID NO: 44 - DNA, 20 bp, synthetic construct
  source 1..20: mol_type = other DNA
ctcttacctgtaccataacc

SEQ ID NO: 45 - DNA, 20 bp, synthetic construct
  source 1..20: mol_type = other DNA
gtatctgtaggaggagaagt

SEQ ID NO: 46 - DNA, 20 bp, synthetic construct
  source 1..20: mol_type = other DNA
tgtatctgtaggaggagaag

SEQ ID NO: 47 - DNA, 20 bp, synthetic construct
  source 1..20: mol_type = other DNA
gtccagtttgtatctgtagg

SEQ ID NO: 48 - DNA, 20 bp, synthetic construct
  source 1..20: mol_type = other DNA
aacaaatgtgtcacaaagta

SEQ ID NO: 49 - DNA, 20 bp, synthetic construct
  source 1..20: mol_type = other DNA
cttcttccccagcccaggta

SEQ ID NO: 50 - DNA, 20 bp, synthetic construct
  source 1..20: mol_type = other DNA
ttcttccccagcccaggtaa

SEQ ID NO: 51 - DNA, 20 bp, synthetic construct
  source 1..20: mol_type = other DNA
agcccaggtaagggcagctt

SEQ ID NO: 52 - DNA, 20 bp, synthetic construct
  source 1..20: mol_type = other DNA
tttcaaaacctgtcagtgat

SEQ ID NO: 53 - DNA, 20 bp, synthetic construct
  source 1..20: mol_type = other DNA
ttcaaaacctgtcagtgatt

SEQ ID NO: 54 - DNA, 20 bp, synthetic construct
  source 1..20: mol_type = other DNA
ccgaatcctcctcctgaaag

SEQ ID NO: 55 - DNA, 20 bp, synthetic construct
  source 1..20: mol_type = other DNA
cttacctgggctggggaaga

SEQ ID NO: 56 - DNA, 20 bp, synthetic construct
  source 1..20: mol_type = other DNA
ttcgtatctgtaaaaccaag

SEQ ID NO: 57 - DNA, 20 bp, synthetic construct
  source 1..20: mol_type = other DNA
ccacacccaaaaggccacac

SEQ ID NO: 58 - DNA, 20 bp, synthetic construct
  source 1..20: mol_type = other DNA
cccaccagctcagctccacg

SEQ ID NO: 59 - DNA, 20 bp, synthetic construct
  source 1..20: mol_type = other DNA
cgctgtcaagtccagttcta

SEQ ID NO: 60 - DNA, 20 bp, synthetic construct
  source 1..20: mol_type = other DNA
gctgtcaagtccagttctac

SEQ ID NO: 61 - DNA, 20 bp, synthetic construct
  source 1..20: mol_type = other DNA
agtccagttctacgggctct

SEQ ID NO: 62 - DNA, 20 bp, synthetic construct
  source 1..20: mol_type = other DNA
cacccagatcgtcagcgccg

SEQ ID NO: 63 - DNA, 20 bp, synthetic construct
  source 1..20: mol_type = other DNA
acctgctctaccccaggcct

SEQ ID NO: 64 - DNA, 20 bp, synthetic construct
  source 1..20: mol_type = other DNA
ccactcacctgctctacccc

SEQ ID NO: 65 - DNA, 20 bp, synthetic construct
  source 1..20: mol_type = other DNA
cacggacccgcagcccctca

SEQ ID NO: 66 - DNA, 20 bp, synthetic construct
  source 1..20: mol_type = other DNA
gcgggggttctgccagaagg

SEQ ID NO: 67 - DNA, 20 bp, synthetic construct
  source 1..20: mol_type = other DNA
gttgcgggggttctgccaga

SEQ ID NO: 68 - DNA, 20 bp, synthetic construct
  source 1..20: mol_type = other DNA
atgacgagtggacccaggat

SEQ ID NO: 69 - DNA, 20 bp, synthetic construct
  source 1..20: mol_type = other DNA
tgacgagtggacccaggata

SEQ ID NO: 70 - DNA, 20 bp, synthetic construct
  source 1..20: mol_type = other DNA
acctgctctaccccaggcct

SEQ ID NO: 71 - DNA, 20 bp, synthetic construct
  source 1..20: mol_type = other DNA
ccaacagtgtcctaccagca

SEQ ID NO: 72 - DNA, 20 bp, synthetic construct
  source 1..20: mol_type = other DNA
caacagtgtcctaccagcaa

SEQ ID NO: 73 - DNA, 20 bp, synthetic construct
  source 1..20: mol_type = other DNA
aacagtgtcctaccagcaag

SEQ ID NO: 74 - DNA, 20 bp, synthetic construct
  source 1..20: mol_type = other DNA
gtctgaaagaaagcagggag

SEQ ID NO: 75 - DNA, 20 bp, synthetic construct
  source 1..20: mol_type = other DNA
ccacagtctgaaagaaagca

SEQ ID NO: 76 - DNA, 20 bp, synthetic construct
  source 1..20: mol_type = other DNA
gccacagtctgaaagaaagc

SEQ ID NO: 77 - DNA, 20 bp, synthetic construct
  source 1..20: mol_type = other DNA
gacactgttggcacggagga

SEQ ID NO: 78 - DNA, 20 bp, synthetic construct
  source 1..20: mol_type = other DNA
gtaggacactgttggcacgg

SEQ ID NO: 79 - DNA, 20 bp, synthetic construct
  source 1..20: mol_type = other DNA
taccatggccatcaacacaa

SEQ ID NO: 80 - DNA, 20 bp, synthetic construct
  source 1..20: mol_type = other DNA
ttaccatggccatcaacaca

SEQ ID NO: 81 - DNA, 20 bp, synthetic construct
  source 1..20: mol_type = other DNA
ccagctcagctccacgtggt

SEQ ID NO: 82 - DNA, 20 bp, synthetic construct
  source 1..20: mol_type = other DNA
cacagacccgcagcccctca

SEQ ID NO: 83 - DNA, 20 bp, synthetic construct
  source 1..20: mol_type = other DNA
gcgggggttctgccagaagg

SEQ ID NO: 84 - DNA, 20 bp, synthetic construct
  source 1..20: mol_type = other DNA
gttgcgggggttctgccaga

SEQ ID NO: 85 - DNA, 20 bp, synthetic construct
  source 1..20: mol_type = other DNA
atgacgagtggacccaggat

SEQ ID NO: 86 - DNA, 20 bp, synthetic construct
  source 1..20: mol_type = other DNA
tgacgagtggacccaggata

SEQ ID NO: 87 - DNA, 20 bp, synthetic construct
  source 1..20: mol_type = other DNA
acctgctctaccccaggcct

SEQ ID NO: 88 - DNA, 20 bp, synthetic construct
  source 1..20: mol_type = other DNA
tcaacagagtcttaccagca

SEQ ID NO: 89 - DNA, 20 bp, synthetic construct
  source 1..20: mol_type = other DNA
caacagagtcttaccagcaa

SEQ ID NO: 90 - DNA, 20 bp, synthetic construct
  source 1..20: mol_type = other DNA
aacagagtcttaccagcaag

SEQ ID NO: 91 - DNA, 20 bp, synthetic construct
  source 1..20: mol_type = other DNA
cacagtctgaaagaaaacag

SEQ ID NO: 92 - DNA, 20 bp, synthetic construct
  source 1..20: mol_type = other DNA
ccacagtctgaaagaaaaca

SEQ ID NO: 93 - DNA, 20 bp, synthetic construct
  source 1..20: mol_type = other DNA
gccacagtctgaaagaaaac

SEQ ID NO: 94 - DNA, 20 bp, synthetic construct
  source 1..20: mol_type = other DNA
tccaggcatgcagatcccac

SEQ ID NO: 95 - DNA, 20 bp, synthetic construct
  source 1..20: mol_type = other DNA
tgcagatcccacaggcgccc

SEQ ID NO: 96 - DNA, 20 bp, synthetic construct
  source 1..20: mol_type = other DNA
cgactggccagggcgcctgt

SEQ ID NO: 97 - DNA, 20 bp, synthetic construct
  source 1..20: mol_type = other DNA
acgactggccagggcgcctg

SEQ ID NO: 98 - DNA, 20 bp, synthetic construct
  source 1..20: mol_type = other DNA
accgcccagacgactggcca

SEQ ID NO: 99 - DNA, 20 bp, synthetic construct
  source 1..20: mol_type = other DNA
caccgcccagacgactggcc

SEQ ID NO: 100 - DNA, 20 bp, synthetic construct
  source 1..20: mol_type = other DNA
tgtagcaccgcccagacgac

SEQ ID NO: 101 - DNA, 20 bp, synthetic construct
  source 1..20: mol_type = other DNA
gggcggtgctacaactgggc

SEQ ID NO: 102 - DNA, 20 bp, synthetic construct
  source 1..20: mol_type = other DNA
cggtgctacaactgggctgg

SEQ ID NO: 103 - DNA, 20 bp, synthetic construct
  source 1..20: mol_type = other DNA
ctacaactgggctggcggcc

SEQ ID NO: 104 - DNA, 20 bp, synthetic construct
  source 1..20: mol_type = other DNA
cacctacctaagaaccatcc

SEQ ID NO: 105 - DNA, 20 bp, synthetic construct
  source 1..20: mol_type = other DNA
ggggttccagggcctgtctg

SEQ ID NO: 106 - DNA, 20 bp, synthetic construct
  source 1..20: mol_type = other DNA
gggggttccagggcctgtct

SEQ ID NO: 107 - DNA, 20 bp, synthetic construct
  source 1..20: mol_type = other DNA
ggggggttccagggcctgtc

SEQ ID NO: 108 - DNA, 20 bp, synthetic construct
  source 1..20: mol_type = other DNA
cagcaaccagacggacaagc

SEQ ID NO: 109 - DNA, 20 bp, synthetic construct
  source 1..20: mol_type = other DNA
cccgaggaccgcagccagcc

SEQ ID NO: 110 - DNA, 20 bp, synthetic construct
  source 1..20: mol_type = other DNA
ggaccgcagccagcccggcc

SEQ ID NO: 111 - DNA, 20 bp, synthetic construct
  source 1..20: mol_type = other DNA
cgtgtcacacaactgcccaa

SEQ ID NO: 112 - DNA, 20 bp, synthetic construct
  source 1..20: mol_type = other DNA
gtgtcacacaactgcccaac

SEQ ID NO: 113 - DNA, 20 bp, synthetic construct
  source 1..20: mol_type = other DNA
cgcagatcaaagagagcctg

SEQ ID NO: 114 - DNA, 20 bp, synthetic construct
  source 1..20: mol_type = other DNA
gcagatcaaagagagcctgc

SEQ ID NO: 115 - DNA, 20 bp, synthetic construct
  source 1..20: mol_type = other DNA
agccggccagttccaaaccc

SEQ ID NO: 116 - DNA, 20 bp, synthetic construct
  source 1..20: mol_type = other DNA
cggccagttccaaaccctgg

SEQ ID NO: 117 - DNA, 20 bp, synthetic construct
  source 1..20: mol_type = other DNA
cagttccaaaccctggtggt

SEQ ID NO: 118 - DNA, 20 bp, synthetic construct
  source 1..20: mol_type = other DNA
ggacccagactagcagcacc

SEQ ID NO: 119 - DNA, 20 bp, synthetic construct
  source 1..20: mol_type = other DNA
cacctacctaagaaccatcc

SEQ ID NO: 120 - DNA, 20 bp, synthetic construct
  source 1..20: mol_type = other DNA
ggagtctgagagatggagag

SEQ ID NO: 121 - DNA, 20 bp, synthetic construct
  source 1..20: mol_type = other DNA
tctggaagggcacaaaggtc

SEQ ID NO: 122 - DNA, 20 bp, synthetic construct
  source 1..20: mol_type = other DNA
ttctctctggaagggcacaa

SEQ ID NO: 123 - DNA, 20 bp, synthetic construct
  source 1..20: mol_type = other DNA
tgacgttacctcgtgcggcc

SEQ ID NO: 124 - DNA, 20 bp, synthetic construct
  source 1..20: mol_type = other DNA
tccctgcagagaaacacact

SEQ ID NO: 125 - DNA, 20 bp, synthetic construct
  source 1..20: mol_type = other DNA
gagactcaccaggggctggc

SEQ ID NO: 126 - DNA, 20 bp, synthetic construct
  source 1..20: mol_type = other DNA
tctttgaggagaaagggaga

SEQ ID NO: 127 - DNA, 20 bp, synthetic construct
  source 1..20: mol_type = other DNA
ttctttgaggagaaagggag

SEQ ID NO: 128 - DNA, 20 bp, synthetic construct
  source 1..20: mol_type = other DNA
tcagggttctggatatctgt

SEQ ID NO: 129 - DNA, 20 bp, synthetic construct
  source 1..20: mol_type = other DNA
ctctcagctggtacacggca

SEQ ID NO: 130 - DNA, 20 bp, synthetic construct
  source 1..20: mol_type = other DNA
agctggtacacggcagggtc

SEQ ID NO: 131 - DNA, 20 bp, synthetic construct
  source 1..20: mol_type = other DNA
acacggcagggtcagggttc

SEQ ID NO: 132 - DNA, 20 bp, synthetic construct
  source 1..20: mol_type = other DNA
gagaatcaaaatcggtgaat

SEQ ID NO: 133 - DNA, 20 bp, synthetic construct
  source 1..20: mol_type = other DNA
aacaaatgtgtcacaaagta

SEQ ID NO: 134 - DNA, 20 bp, synthetic construct
  source 1..20: mol_type = other DNA
tgtgctagacatgaggtcta

SEQ ID NO: 135 - DNA, 2805 bp, unidentified
  source 1..2805: mol_type = unassigned DNA
ccgtggccttagctgtgctcgcgctactctctctttctggcctggaggtaattcattcattcatccatccattcgttcattcggtttactgagtacctactatgtgccagcccctgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggacgagatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaatgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagccgtggccttagctgtgctcgcgctactctctctttctggcctggagg

SEQ ID NO: 136 - DNA, 2709 bp, unidentified
  source 1..2709: mol_type = unassigned DNA
taattcattcattcatccatccattcgttcattcggtttactgagtacctactatgtgccagcccctgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggacgagatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaatgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaag

SEQ ID NO: 137 - RNA, 20 nt, synthetic construct
  source 1..20: mol_type = other RNA
gagccccaaggtaaaaaggc

SEQ ID NO: 138 - RNA, 20 nt, synthetic construct
  source 1..20: mol_type = other RNA
agccccaaggtaaaaaggcc

SEQ ID NO: 139 - RNA, 20 nt, synthetic construct
  source 1..20: mol_type = other RNA
cagctcacagtgtgccacca

SEQ ID NO: 140 - RNA, 20 nt, synthetic construct
  source 1..20: mol_type = other RNA
tatgaccagatggacctggc

SEQ ID NO: 141 - RNA, 20 nt, synthetic construct
  source 1..20: mol_type = other RNA
actggaccagtatgtcttcc

SEQ ID NO: 142 - RNA, 20 nt, synthetic construct
  source 1..20: mol_type = other RNA
tgtcttccaggactcccagc

SEQ ID NO: 143 - RNA, 20 nt, synthetic construct
  source 1..20: mol_type = other RNA
cttccaggactcccagctgg

SEQ ID NO: 144 - RNA, 20 nt, synthetic construct
  source 1..20: mol_type = other RNA
ttccaggactcccagctgga

SEQ ID NO: 145 - RNA, 20 nt, synthetic construct
  source 1..20: mol_type = other RNA
ttccagtgcttcaggtctgc

SEQ ID NO: 146 - RNA, 20 nt, synthetic construct
  source 1..20: mol_type = other RNA
ttcaaccaggagccagcctc

SEQ ID NO: 147 - RNA, 20 nt, synthetic construct
  source 1..20: mol_type = other RNA
gaccagattcccagtatgtt

SEQ ID NO: 148 - RNA, 20 nt, synthetic construct
  source 1..20: mol_type = other RNA
accagattcccagtatgtta

SEQ ID NO: 149 - RNA, 20 nt, synthetic construct
  source 1..20: mol_type = other RNA
ctctggcaaatctctgaggc

SEQ ID NO: 150 - RNA, 20 nt, synthetic construct
  source 1..20: mol_type = other RNA
agccaagtaccccctcccag

SEQ ID NO: 151 - RNA, 20 nt, synthetic construct
  source 1..20: mol_type = other RNA
acctcccgagcaaacatgac

SEQ ID NO: 152 - RNA, 20 nt, synthetic construct
  source 1..20: mol_type = other RNA
cccacccaatgcccggcagc

SEQ ID NO: 153 - RNA, 20 nt, synthetic construct
  source 1..20: mol_type = other RNA
aggccattttggaagcttgt

SEQ ID NO: 154 - RNA, 20 nt, synthetic construct
  source 1..20: mol_type = other RNA
tggtgcaggccaggctggag

SEQ ID NO: 155 - RNA, 20 nt, synthetic construct
  source 1..20: mol_type = other RNA
gaacggcagctggcccaagg

SEQ ID NO: 156 - RNA, 20 nt, synthetic construct
  source 1..20: mol_type = other RNA
ggcccaaggaggcctggctg

SEQ ID NO: 157 - RNA, 20 nt, synthetic construct
  source 1..20: mol_type = other RNA
gacacgagtgattgctgtgc

SEQ ID NO: 158 - RNA, 20 nt, synthetic construct
  source 1..20: mol_type = other RNA
acacgagtgattgctgtgct

SEQ ID NO: 159 - RNA, 20 nt, synthetic construct
  source 1..20: mol_type = other RNA
ctggtcagggcaagagctat

SEQ ID NO: 160 - RNA, 20 nt, synthetic construct
  source 1..20: mol_type = other RNA
tggtcagggcaagagctatt

SEQ ID NO: 161 - RNA, 20 nt, synthetic construct
  source 1..20: mol_type = other RNA
ttccagaagaagctgctccg

SEQ ID NO: 162 - RNA, 20 nt, synthetic construct
  source 1..20: mol_type = other RNA
cagacatcaaagtaccctac

SEQ ID NO: 163 - RNA, 20 nt, synthetic construct
  source 1..20: mol_type = other RNA
acatcaaagtaccctacagg

SEQ ID NO: 164 - RNA, 20 nt, synthetic construct
  source 1..20: mol_type = other RNA
cgcccaggtcctcacgtctg

SEQ ID NO: 165 - RNA, 20 nt, synthetic construct
  source 1..20: mol_type = other RNA
cttagtccaacacccaccgc

SEQ ID NO: 166 - RNA, 20 nt, synthetic construct
  source 1..20: mol_type = other RNA
ggaagcagaaggtgcttgcg

SEQ ID NO: 167 - RNA, 20 nt, synthetic construct
  source 1..20: mol_type = other RNA
ggctgcagccggggacactg

SEQ ID NO: 168 - RNA, 20 nt, synthetic construct
  source 1..20: mol_type = other RNA
gctgcagccggggacactgc

SEQ ID NO: 169 - RNA, 20 nt, synthetic construct
  source 1..20: mol_type = other RNA
ctgccaaattccagcctcct

SEQ ID NO: 170 - RNA, 20 nt, synthetic construct
  source 1..20: mol_type = other RNA
ggcgggccaagacttctccc

SEQ ID NO: 171 - RNA, 20 nt, synthetic construct
  source 1..20: mol_type = other RNA
tgtgcagactcagaggtgag

SEQ ID NO: 172 - RNA, 20 nt, synthetic construct
  source 1..20: mol_type = other RNA
agactcagaggtgagaggag

SEQ ID NO: 173 - RNA, 20 nt, synthetic construct
  source 1..20: mol_type = other RNA
ctcagaggtgagaggagagg

SEQ ID NO: 174 - RNA, 20 nt, synthetic construct
  source 1..20: mol_type = other RNA
cgtccagtacaacaagttca

SEQ ID NO: 175 - RNA, 20 nt, synthetic construct
  source 1..20: mol_type = other RNA
ttttaccttggggctctgac

SEQ ID NO: 176 - RNA, 20 nt, synthetic construct
  source 1..20: mol_type = other RNA
ttctgggaggaaaagtccct

SEQ ID NO: 177 - RNA, 20 nt, synthetic construct
  source 1..20: mol_type = other RNA
tactgaaaatgtccttgctc

SEQ ID NO: 178 - RNA, 20 nt, synthetic construct
  source 1..20: mol_type = other RNA
cacctggcttccagtgcttc

SEQ ID NO: 179 - RNA, 20 nt, synthetic construct
  source 1..20: mol_type = other RNA
gggctcagctgtgaggaagt

SEQ ID NO: 180 - RNA, 20 nt, synthetic construct
  source 1..20: mol_type = other RNA
taacatactgggaatctggt

SEQ ID NO: 181 - RNA, 20 nt, synthetic construct
  source 1..20: mol_type = other RNA
cttacctgtcatgtttgctc

SEQ ID NO: 182 - RNA, 20 nt, synthetic construct
  source 1..20: mol_type = other RNA
ccttacctgtcatgtttgct

SEQ ID NO: 183 - RNA, 20 nt, synthetic construct
  source 1..20: mol_type = other RNA
tgctctggagatggagaagc

SEQ ID NO: 184 - RNA, 20 nt, synthetic construct
  source 1..20: mol_type = other RNA
accggctctgcaaaggccag

SEQ ID NO: 185 - RNA, 100 nt, synthetic construct
  source 1..100: mol_type = other RNA
  modified_base 1: mod_base = cm
  modified_base 2: mod_base = gm
  modified_base 98..99: mod_base = um
  modified_base 1^2: mod_base = OTHER; note = Phosphorothioate bond
  modified_base 2^3: mod_base = OTHER; note = Phosphorothioate bond
  modified_base 98^99: mod_base = OTHER; note = Phosphorothioate bond
  modified_base 99^100: mod_base = OTHER; note = Phosphorothioate bond
cgcgagcacagctaaggccagttttagagctagaaatagcaagttaaaataaggctagtccgttatcaacttgaaaaagtggcaccgagtcggtgctttt

SEQ ID NO: 186 - RNA, 100 nt, synthetic construct
  source 1..100: mol_type = other RNA
  modified_base 1: mod_base = OTHER; note = 2-O Methyl Adenosine
  modified_base 2: mod_base = cm
  modified_base 98..99: mod_base = um
  modified_base 1^2: mod_base = OTHER; note = Phosphorothioate bond
  modified_base 2^3: mod_base = OTHER; note = Phosphorothioate bond
  modified_base 98^99: mod_base = OTHER; note = Phosphorothioate bond
  modified_base 99^100: mod_base = OTHER; note = Phosphorothioate bond
actctctctttctggcctgggttttagagctagaaatagcaagttaaaataaggctagtccgttatcaacttgaaaaagtggcaccgagtcggtgctttt

SEQ ID NO: 187 - RNA, 20 nt, synthetic construct
  source 1..20: mol_type = other RNA
gcccgaatgctgtcagcttc

SEQ ID NO: 188 - RNA, 20 nt, synthetic construct
  source 1..20: mol_type = other RNA
ggccacggagcgagacatct

SEQ ID NO: 189 - RNA, 20 nt, synthetic construct
  source 1..20: mol_type = other RNA
cgcgagcacagctaaggcca

SEQ ID NO: 190 - RNA, 20 nt, synthetic construct
  source 1..20: mol_type = other RNA
gagtagcgcgagcacagcta

SEQ ID NO: 191 - RNA, 20 nt, synthetic construct
  source 1..20: mol_type = other RNA
ggccgagatgtctcgctccg

SEQ ID NO: 192 - RNA, 20 nt, synthetic construct
  source 1..20: mol_type = other RNA
ctcgcgctactctctctttc

SEQ ID NO: 193 - RNA, 20 nt, synthetic construct
  source 1..20: mol_type = other RNA
gctactctctctttctggcc

SEQ ID NO: 194 - RNA, 20 nt, synthetic construct
  source 1..20: mol_type = other RNA
actctctctttctggcctgg

SEQ ID NO: 195 - protein, 1581 aa, synthetic construct
PKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV